(19) United States
(12) Patent Application Publication
(10) Pub. No.: US 2008/0076120 A1
(43) Pub. Date: Mar. 27, 2008

(54) Methods for the identification, evaluation and treatment of patients having CC-Chemokine receptor 2 (CCR-2) mediated disorders

(75) Inventors: Debra D. Donaldson, Medford, MA (US); Alexander N. Parker, Framingham, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/522,273

(22) Filed: Sep. 14, 2006

(51) Int. Cl. C12Q 1/68

(52) U.S. Cl. 435/6

(57) ABSTRACT

The present invention is directed to the identification of predictive genotypes, e.g., predictive single nucleotide polymorphisms (SNPs), and markers that can be used to determine whether a patient having a CC-Chemokine Receptor 2 (CCR-2) mediated disorders is likely to be responsive or non-responsive to a therapeutic regimen. For example, the present invention is directed, in part, to the use of certain individual and/or combinations of SNPs, wherein the expression of particular alleles at particular SNPs, or combinations of alleles at loci in linkage disequilibrium with a particular SNP, correlate with responsiveness or non-responsiveness to a therapeutic regimen. The present invention is also directed to the use of certain individual and/or combinations of predictive markers which correlate with responsiveness or non-responsiveness to a therapeutic regimen. Thus, by examining allelic expression at particular SNPs, combinations of alleles at loci in linkage disequilibrium with a particular SNP, or expression levels of individual predictive markers and/or predictive markers comprising a marker set, it is possible to determine whether a patient having a CCR-2 mediated disorder will likely respond or not respond to a therapeutic regimen.

### BACKGROUND OF THE INVENTION

CC-Chemokine Receptor 2 (CCR-2) is expressed on the surface of several leukocyte subsets, such as monocytes, dendritic cells and memory T-cells, and appears to be expressed in two slightly different forms (CCR-2α and CCR-2β) due to alternative splicing of the mRNA encoding the carboxy-terminal region (Charo et al., Proc. Natl. Acad. Sci. USA 91:2752-2756 (1994)). CCR-2 is the primary receptor for Chemokine Ligand CCL2 (Monocyte Chemoattractant Protein 1 (MCP-1)) and also binds CCL8 (MCP-2), CCL7 (MCP-3), CCL13 (MCP-4), CCL16 (HCC4) and HIV.

A substantial body of evidence, in both animal models and man, supports the involvement of CCR-2 and CCL2 in the pathogenesis of atherosclerosis and other CCR-2 mediated disorders. CCL2 is expressed in atherosclerotic lesions in human arteries and is associated with macrophage-rich regions (Nelken, N. A. et al. (1991) J. Clin. Invest. 88(4):1121-7; Yla-Hertuala S. et al. (1991) Prot. Natl. Aacad. Sci USA. 88(12):5252-6; and Seino Y. et al. (1995) Cytokine 7(6):575-9). In animals, elimination of CCR-2 expression by genetic manipulation in knockout models results in reduced atheroma generation in apolipoprotein E (ApoE) deletion mouse models of atherosclerosis (Boring L. et al. (1998) Nature 394(6696):894-7; and Dawson T. C. et al. (1999) Atherosclerosis 143(1):205-11). Similarly, genetic disruption of the gene encoding CCL2, truncation of CCL2, or expression of dominant negative CCL2 reduced atherosclerotic lesion formation in multiple mouse models of atherosclerosis (Ni W. et al. (2001) Circulation 103(16):2096-2101; Gu L. et al. (1998) Mol. Cell 2(2):275-81; Gosling J. et al. (1999) J. Clin. Invest. 103(6):773-778; and Inoue S. et al. (2002) Circulation 106(21):2700-6). Alternatively, overexpression of CCL2, either locally in the carotid artery (Namiki M. et al. (2002) Arterioscler. Thromb. Vasc. Biol. 22(1):115-20) or in blood monocytes derived from transplanted transgenic overexpressing bone marrow from syngeneic mice (Aiello R. J. et al. (1999) Arterioscler. Thromb. Vasc. Biol. 19(6):1518-25), drives increased atheroma formation in susceptible animal models.

Thus, CCR-2 antagonists, CCL2 antagonists or agents which interfere with the binding of CCR-2 to its natural ligands represent a class of important therapeutic agents.

As described above, CCR-2 and its natural ligands represent pharmaceutically important proteins. However, natural genetic variability exists between individuals in every population with respect to such proteins. Such genetic variability can be manifested as single nucleotide polymorphisms (SNPs) within the genes which encode these proteins. While most SNPs have not been associated with any particular disease phenotype, some SNPs have been associated with disease susceptibility (Ulbrecht M et al. (2000) Am. J. Respir. Crit. Care Med. 161: 469-74) and drug response (Wolfe C R et al. (2000) BMJ 320:987-90). The ability to predict whether a patient will respond to a drug based on the person's genotype has numerous advantages, such as eliminating drugs that would prove ineffective for that patient, thereby reducing treatment time; possibly reducing side effects; possibly reducing dosage; improving the efficiency of several steps in the drug discovery and drug development processes; and likely reducing the failure rate of candidate and approved drugs.

### SUMMARY OF THE INVENTION

The present invention is based, in part, on the discovery that over-expression of a CCL2 biomarker, e.g., that correlates with an individual's genotype of the CCL2 genomic sequence at a pre-selected nucleotide, or one or more nucleotides, alleles or combination of alleles in linkage disequilibrium with the pre-selected nucleotide, as compared to a reference standard, can be used to determine whether a patient, e.g., a human patient, having a CCR-2 mediated disorder, e.g., a CCR-2 mediated disorder as described herein, will be responsive or non-responsive to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. It was discovered that a genotype at a pre-selected nucleotide within the CCL2 genomic sequence of a patient having or at risk of having a CCR-2 mediated disorder, or at one or more nucleotides in linkage disequilibrium with the pre-selected nucleotide, can be used to determine whether the patient will be responsive or non-responsive to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. Thus, for example, the compositions and methods provided herein can be used to determine whether a patient will be responsive or non-responsive to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. Based on these identifications, the present invention provides, without limitation: 1) methods and compositions for evaluating a patient having a CCR-2 mediated disorder, e.g., a CCR-2 mediated disorder as described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof; 2) methods and compositions for selecting a treatment regimen for a patient having a CCR-2 mediated disorder, e.g., a CCR-2 mediated disorder as described herein; 3) methods for selecting a patient population having a CCR-2 mediated disorder, e.g., a CCR-2 mediated disorder as described herein, for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof; 4) methods and compositions for treatment of a patient having a CCR-2 mediated disorder, e.g., a CCR-2 mediated disorder as described herein; 5) methods and compositions for monitoring the effectiveness of a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, used for the treatment of a patient having a CCR-2 mediated disorder, e.g., a CCR-2 mediated disorder as described herein; 6) methods for processing approval of payment or processing of payment for a treatment regimen of a patient having a CCR-2 mediated disorder, e.g., a CCR-2 mediated disorder as described herein; 7) arrays and kits for evaluating a patient having a CCR-2 mediated disorder, e.g., a CCR-2 mediated disorder as described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof; and 8) an article of manufacture as described herein.

In one aspect, the invention features a method of evaluating a patient having a CCR-2 mediated disorder, e.g., a CCR-2 mediated disorder described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. The method includes determining or confirming a value for a parameter related to a patient's CCL2 expression level, recording the value, and determining, recommending or selecting an appropriate treatment regimen based upon the value. In one embodiment, the method includes, e.g., an embodiment or embodiments described herein.

In another aspect, the invention features a method of evaluating a patient having a CCR-2 mediated disorder, e.g., a CCR-2 mediated disorder described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. The method includes determining or confirming a patient's genotype at a pre-selected nucleotide within the patient's CCL2 genomic sequence, recording the patient's genotype, and determining, recommending or selecting an appropriate treatment regimen based upon the patient's genotype. In one embodiment, the method includes, e.g., an embodiment or embodiments described herein.

In another aspect, the invention features a method of evaluating a patient having a CCR-2 mediated disorder, e.g., a CCR-2 mediated disorder described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. The method includes determining or confirming a value for a parameter related to a patient's CCL2 expression level, recording the value, and determining whether to begin, continue, discontinue, change or alter the treatment regimen based upon the value. In one embodiment, the method includes, e.g., an embodiment or embodiments described herein.

In another aspect, the invention features a method of evaluating a patient having a CCR-2 mediated disorder, e.g., a CCR-2 mediated disorder described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. The method includes determining or confirming a patient's genotype at a pre-selected nucleotide within the patient's CCL2 genomic sequence, recording the patient's genotype, and determining whether to begin, continue, discontinue, change or alter the treatment regimen based upon the patient's genotype. In one embodiment, the method includes, e.g., an embodiment or embodiments described herein.

In another aspect, the invention features a method for selecting a treatment regimen for a patient having a CCR-2 mediated disorder, e.g., a CCR-2 mediated disorder described herein. The method includes determining or confirming a value for a parameter related to a patient's CCL2 expression level, recording the value, and selecting an appropriate treatment regimen based upon the value. In one embodiment, the method includes, e.g., an embodiment or embodiments described herein.

In another aspect, the invention features a method for selecting a treatment regimen for a patient having a CCR-2 mediated disorder, e.g., a CCR-2 mediated disorder described herein. The method includes determining a patient's genotype at a pre-selected nucleotide within the patient's CCL2 genomic sequence, recording the patient's genotype, and selecting an appropriate treatment regimen based upon the patient's genotype. In one embodiment, the method includes, e.g., an embodiment or embodiments described herein.

In another aspect, the invention features a method for selecting a patient population for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. The method includes providing a population of patients having a CCR-2 mediated disorder, e.g., a CCR-2 mediated disorder described herein, determining a patient's CCL2 expression level, and selecting one or more patients for treatment that have increased CCL2 expression levels in comparison to a reference standard, to thereby select a patient population for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. In one embodiment, the method includes, e.g., an embodiment or embodiments described herein.

In another aspect, the invention features a method for selecting a patient population for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. The method includes providing a population of patients having a CCR-2 mediated disorder, e.g., a CCR-2 mediated disorder described herein, determining a patient's genotype at a pre-selected nucleotide within the patient's CCL2 genomic sequence, and selecting one or more patients based upon the patient(s) having a genotype which predisposes the patient(s) to being responsive to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, to thereby select a patient population for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. In one embodiment, the method includes, e.g., an embodiment or embodiments described herein.

In another aspect, the invention features a method for selecting a patient population for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. The method includes providing a population of patients having a CCR-2 mediated disorder, e.g., a CCR-2 mediated disorder described herein, and determining the genotype at a nucleotide at position 2485, as numbered in SEQ ID NO:1, or at a nucleotide, allele, or combination of alleles in linkage disequilibrium with the nucleotide at position 2485, in one or more patients. In one embodiment, the method includes, e.g., an embodiment or embodiments described herein.

In another aspect, the invention features a method for treating a patient having a CCR-2 mediated disorder, e.g., a CCR-2 mediated disorder described herein. The method includes selecting a patient who over-expresses CCL2 as compared to a reference standard and administering to the patient a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, to thereby treat the CCR-2 mediated disorder. In one embodiment, the method includes, e.g., an embodiment or embodiments described herein.

In another aspect, the invention features a method for treating a patient having a CCR-2 mediated disorder, e.g., a CCR-2 mediated disorder described herein. In one embodiment, the method includes determining or confirming a value for a parameter related to a patient's CCL2 expression level and administering to the patient a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, to thereby treat the CCR-2 mediated disorder. In one embodiment, the method includes, e.g., an embodiment or embodiments described herein.

In another aspect, the invention features the use of a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, in the manufacture of a medicament for the treatment of a CCR-2 mediated disorder, e.g., a CCR-2 mediated disorder as described herein, in a patient who over-expresses CCL2 as compared to a reference standard. In one embodiment, the use includes, e.g., an embodiment or embodiments described herein.

In another aspect, the invention features the use of a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, in the manufacture of a medicament for the treatment of a CCR-2 mediated disorder, e.g., a CCR-2 mediated disorder as described herein, in a patient being selected on the criterion of over-expressing CCL2 as compared to a reference standard. In one embodiment, the use includes, e.g., an embodiment or embodiments described herein.

In another aspect, the invention features a method for treating a patient having a CCR-2 mediated disorder, e.g., a CCR-2 mediated disorder described herein. The method includes determining or confirming a patient's genotype of a nucleotide at position 2485 as numbered in SEQ ID NO:1, and administering to the patient a treatment regimen based upon the patient's genotype. In one embodiment, the method includes, e.g., an embodiment or embodiments described herein.

In another aspect, the invention features a method for treating a patient having a CCR-2 mediated disorder, e.g., a CCR-2 mediated disorder described herein. In one embodiment, the method includes determining or confirming a patient's genotype at a pre-selected nucleotide within the patient's CCL2 genomic sequence, and administering to the patient a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, to thereby treat the CCR-2 mediated disorder. In one embodiment, the method includes, e.g., an embodiment or embodiments described herein.

In another aspect, the invention features a method for treating a patient having a CCR-2 mediated disorder, e.g., a CCR-2 mediated disorder described herein. In one embodiment, the method includes determining or confirming a patient's genotype at a pre-selected nucleotide within the patient's CCL2 genomic sequence, and administering to the patient a treatment regimen comprising: i) a treatment regimen other than a CCR-2 antagonist treatment regimen, or ii) a treatment regimen other than a CCL2 antagonist treatment, to thereby treat the CCR-2 mediated disorder. In one embodiment, the method includes, e.g., an embodiment or embodiments described herein.

In another aspect, the invention features a method for treating a patient having a CCR-2 mediated disorder, e.g., a CCR-2 mediated disorder described herein. In one embodiment, the method includes selecting a patient based upon the patient having a G at a nucleotide at position 2485 as numbered in SEQ ID NO:1, or a nucleotide, allele or combination of alleles at loci in linkage disequilibrium with the nucleotide at position 2485 as numbered in SEQ ID NO:1, and administering to the patient a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, to thereby treat the CCR-2 mediated disorder. In one embodiment, the method includes, e.g., an embodiment or embodiments described herein.

In one embodiment, the invention features the use of a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, in the manufacture of a medicament for the treatment of a CCR-2 mediated disorder, e.g., a CCR-2 mediated disorder as described herein, in a patient having a G at a nucleotide at position 2485 as numbered in SEQ ID NO:1, or a nucleotide, allele or combination of alleles at loci in linkage disequilibrium with the nucleotide at position 2485 as numbered in SEQ ID NO:1. In one embodiment, the use includes, e.g., an embodiment or embodiments described herein.

In one embodiment, the invention features the use of a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, in the manufacture of a medicament for the treatment of a CCR-2 mediated disorder, e.g., a CCR-2 mediated disorder as described herein, in a patient being selected on the criterion of having a G at a nucleotide at position 2485 as numbered in SEQ ID NO:1, or a nucleotide, allele or combination of alleles at loci in linkage disequilibrium with the nucleotide at position 2485 as numbered in SEQ ID NO:1. In one embodiment, the use includes, e.g., an embodiment or embodiments described herein.

In another aspect, the invention features a method for treating a patient having a CCR-2 mediated disorder, e.g., a CCR-2 mediated disorder described herein. The method includes selecting a patient based upon the patient having a homozygous A allele at a nucleotide at position 2485 as numbered in SEQ ID NO:1, or a nucleotide, allele or combination of alleles at loci in linkage disequilibrium with the nucleotide at position 2485 as numbered in SEQ ID NO:1, and administering to the patient a treatment regimen comprising: i) a treatment regimen other than a CCR-2 antagonist; ii) a treatment regimen other than a CCL2 antagonist; iii) a treatment regimen comprising a CCR-2 antagonist, e.g., a CCR-2 antagonist described herein, in combination with an agent other than a CCR-2 antagonist; iv) a treatment regimen comprising a CCL2 antagonist, e.g., a CCL2 antagonist described herein, in combination with an agent other than a CCL2 antagonist; v) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder; or vi) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder. Alternatively, the patient having a homozygous A allele at a nucleotide at position 2485 as numbered in SEQ ID NO:1, or a nucleotide, allele or combination of alleles at loci in linkage disequilibrium with the nucleotide at position 2485 as numbered in SEQ ID NO:1, is not administered a treatment described herein. In one embodiment, the method includes, e.g., an embodiment or embodiments described herein.

In one embodiment, the invention features the use of a treatment regimen comprising: i) a treatment regimen other than a CCR-2 antagonist; ii) a treatment regimen other than a CCL2 antagonist; iii) a treatment regimen comprising a CCR-2 antagonist, e.g., a CCR-2 antagonist described herein, in combination with an agent other than a CCR-2 antagonist; iv) a treatment regimen comprising a CCL2 antagonist, e.g., a CCL2 antagonist described herein, in combination with an agent other than a CCL2 antagonist; v) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder; or vi) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder, in the manufacture of a medicament for the treatment of a CCR-2 mediated disorder, e.g., a CCR-2 mediated disorder as described herein, in a patient having an A at a nucleotide at position 2485 as numbered in SEQ ID NO:1, or a nucleotide, allele or combination of alleles at loci in linkage disequilibrium with the nucleotide at position 2485 as numbered in SEQ ID NO:1. In one embodiment, the use includes, e.g., an embodiment or embodiments described herein.

In one embodiment, the invention features the use of a treatment regimen comprising: i) a treatment regimen other than a CCR-2 antagonist; ii) a treatment regimen other than a CCL2 antagonist; iii) a treatment regimen comprising a CCR-2 antagonist, e.g., a CCR-2 antagonist described herein, in combination with an agent other than a CCR-2 antagonist; iv) a treatment regimen comprising a CCL2 antagonist, e.g., a CCL2 antagonist described herein, in combination with an agent other than a CCL2 antagonist; v) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder; or vi) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder, in the manufacture of a medicament for the treatment of a CCR-2 mediated disorder, e.g., a CCR-2 mediated disorder as described herein, in a patient being selected on the criterion of having an A at a nucleotide at position 2485 as numbered in SEQ ID NO:1, or a nucleotide, allele or combination of alleles at loci in linkage disequilibrium with the nucleotide at position 2485 as numbered in SEQ ID NO:1. In one embodiment, the use includes, e.g., an embodiment or embodiments described herein.

In another aspect, the invention features a method for monitoring the effectiveness of a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. The method includes determining or confirming a value for a parameter related to a patient's CCL2 expression level at a first time point during or before commencement of the treatment regimen; determining or confirming a value for a parameter related to a patient's CCL2 expression level at a later time point; and comparing both values to assess effectiveness of the treatment regimen. In one embodiment, the method includes, e.g., an embodiment or embodiments described herein.

In another aspect, the invention provides a method of processing approval of payment or processing of payment for a treatment regimen of a patient having a CCR-2 mediated disorder, e.g., a CCR-2 mediated disorder described herein. The method includes reviewing the patient's genotype at a pre-selected nucleotide within the patient's CCL2 genomic sequence for absence or presence of a G allele at a nucleotide at position 2485 as numbered in SEQ ID NO:1, or a nucleotide, allele or combination of alleles at loci in linkage disequilibrium with the nucleotide at position 2485 as numbered in SEQ ID NO:1; making a decision or advising on whether payment should be made for the treatment regimen of the patient based upon the presence or absence of the G allele at the nucleotide at position 2485 as numbered in SEQ ID NO:1, or a nucleotide, allele or combination of alleles at loci in linkage disequilibrium with the nucleotide at position 2485 as numbered in SEQ ID NO:1; and transmitting or recording the decision or advice. In one embodiment, the method includes, e.g., an embodiment or embodiments described herein.

In another aspect, the invention features an array including a substrate having a plurality of addresses. Each address of the plurality includes a unique (as compared to a nucleic acid at another address) nucleic acid (e.g., a DNA or an RNA) which binds a target, wherein at least one of the nucleic acids binds to a CCL2 region and can distinguish between a first CCL2 allele and a second CCL2 allele, wherein the first allele expresses CCL2 at a higher rate than the second. In one embodiment, the array includes, e.g., an embodiment or embodiments described herein.

In another aspect, the invention features a kit for evaluating a patient having a CCR-2 mediated disorder, e.g., a CCR-2 mediated disorder described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, comprising: i) a nucleic acid sequence which hybridizes under stringent conditions to a portion of the genomic sequence of CCL2, or a nucleic acid sequence for amplifying portions of the genomic sequence of CCL2; and ii) instructions for a treatment regimen based upon the presence or absence of a pre-selected nucleotide at position 2485 as numbered in SEQ ID NO:1, or one or more nucleotides in linkage disequilibrium with the nucleotide at position 2485 as numbered in SEQ ID NO:1 in the patient sample. In one embodiment, the kit includes, e.g., an embodiment or embodiments described herein.

In another aspect, the invention features articles of manufacture comprising a pharmaceutical composition comprising a CCR-2 antagonist or a CCL2 antagonist and instructions for determining the appropriateness of use of said composition by determining a patient's genotype, as described herein. In another aspect, the invention features articles of manufacture comprising reagents for making a determination of a patient's genotype, as described herein and instructions for determining the appropriateness of use of a pharmaceutical composition comprising a CCR-2 antagonist or a CCL2 antagonist on the basis of said determination. In one embodiment, the articles of manufacture include, e.g., an embodiment or embodiments described herein.

### DESCRIPTION OF DRAWINGS

FIGS. 1A-K depict a genomic nucleic acid sequence (SEQ ID NO:1) encoding CCL2. There are three exons within this CCL2 gene, which are represented by the capitalized nucleic acids in the boxed regions of the figure. Exon 1 begins at nucleotide 5001 and ends at nucleotide 5149, as numbered in the figure. Exon 2 begins at nucleotide 5946 and ends at nucleotide 6063, as numbered in the figure. Exon 3 begins at nucleotide 6446 and ends at nucleotide 6923, as numbered in the figure. There are two introns within this CCL2 gene. The first intron is located at nucleotide 5150 to 5945, as numbered in the figure. The second intron is located at nucleotide 6064 to 6445, as numbered in the figure. The translation start site begins at nucleic acid 5074, as numbered in the figure, and the translation stop site ends at nucleic acid 6551, as numbered in the figure. The sequence also comprises various functional sequence elements, such as i) four NF-kB sites located at nucleotide 2196 to 2206, 2370 to 2379, 2397 to 2408, and 4923 to 4936, as numbered in the figure; ii) three SP-1 sites located at nucleotide 2445 to 2453, 2481 to 2486, and 4946 to 4958, as numbered in the figure; iii) four AP-1 sites located at nucleotide 2517 to 2525, 2705 to 2714, 4915 to 4924, and 4943 to 4952 as numbered in the figure; iv) one NF-1 site located at nucleotide 4986 to 5003, as numbered in the figure; and v) one CAAT site located at nucleotide 5031 to 5040, as numbered in the figure. The figure also depicts four nucleic acids which are represented by “n” at positions 2236, 2485, 2936 and 5837, as numbered in the figure, which represent variant nucleic acids (or polymorphic sites). For example, the nucleic acid at position 2236 as numbered in the figure can be A or C and has been assigned dbSNP accession number rs2857654 (refer to www.ncbi.nlm.nih.gov/SNP/snp_ref.cgi?rs=2857654). This variant is also referred to as MCP-1−2835A. The nucleic acid at position 2485 as numbered in the figure can be G or A and has been assigned dbSNP accession number rs1024611 (refer to www.ncbi.nlm.nih.gov/SNP/snp_ref.cgi?rs=1024611). This variant is also referred to as MCP-1−2578A. The nucleic acid at position 2936 as numbered in the figure can be A or T and has been assigned dbSNP accession number rs1024610 (refer to www.ncbi.nlm.nih.gov/SNP/snp_ref.cgi?rs=1024610). This variant is also referred to as MCP-1−2136-T. The nucleic acid at position 5837 as numbered in the figure can be G or C and has been assigned dbSNP accession number rs2857657 (refer to www.ncbi.nlm.nih.gov/SNP/snp_ref.cgi?rs=2857657). This variant is also referred to as MCP-1+764G. Additional information on these variants within the CCL2 genomic sequence is also available at www.hapmap.org, which is incorporated herein by reference.

FIGS. 2A-B depict the absolute vs. relative median change of human CRP levels from baseline levels during the course of treatment of patients having atherosclerosis with either placebo or 10 mg/kg MLN1202. These results demonstrate that CRP levels were significantly reduced by 25% (p=0.007) at day 57.

FIG. 3 depicts the correlation between patients' genotype at the polymorphic site at the nucleotide at position 2485, as numbered in SEQ ID NO:1, and baseline median CRP levels in atherosclerotic patients administered a treatment regimen comprising either a placebo or a CCR-2 antagonist, e.g., MLN1202. Results demonstrate that 53% of the study population carried the susceptibility allele for responding to MLN1202.

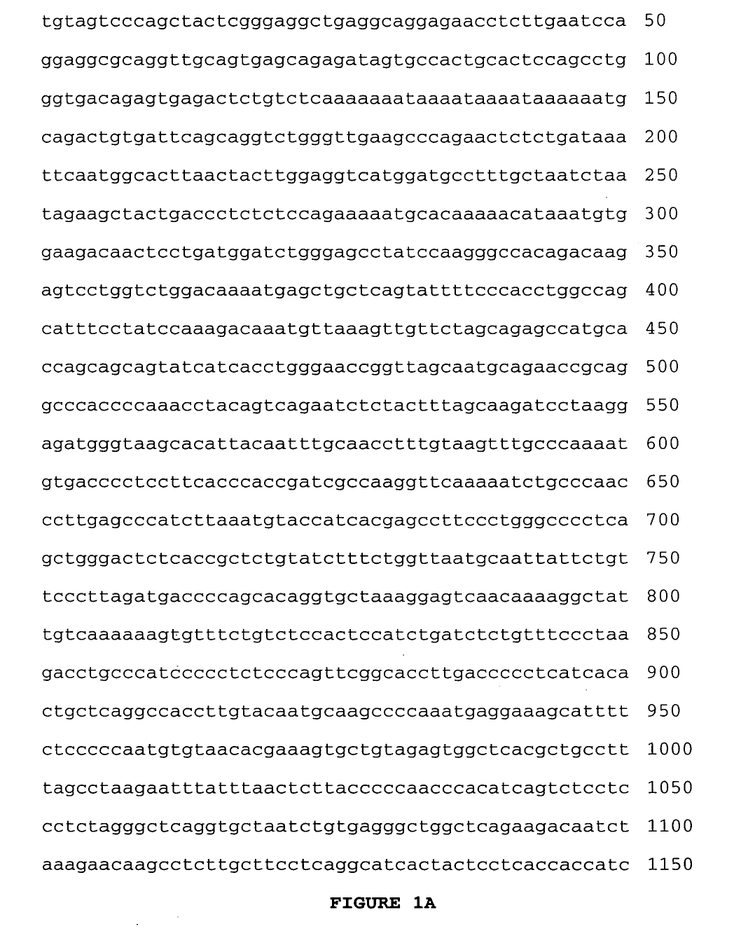

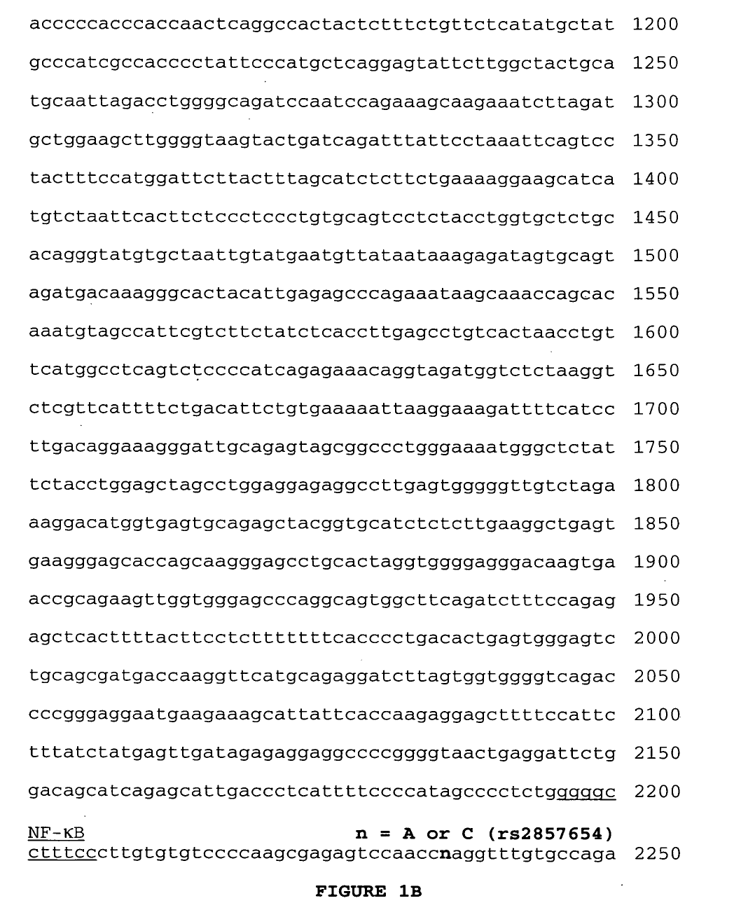

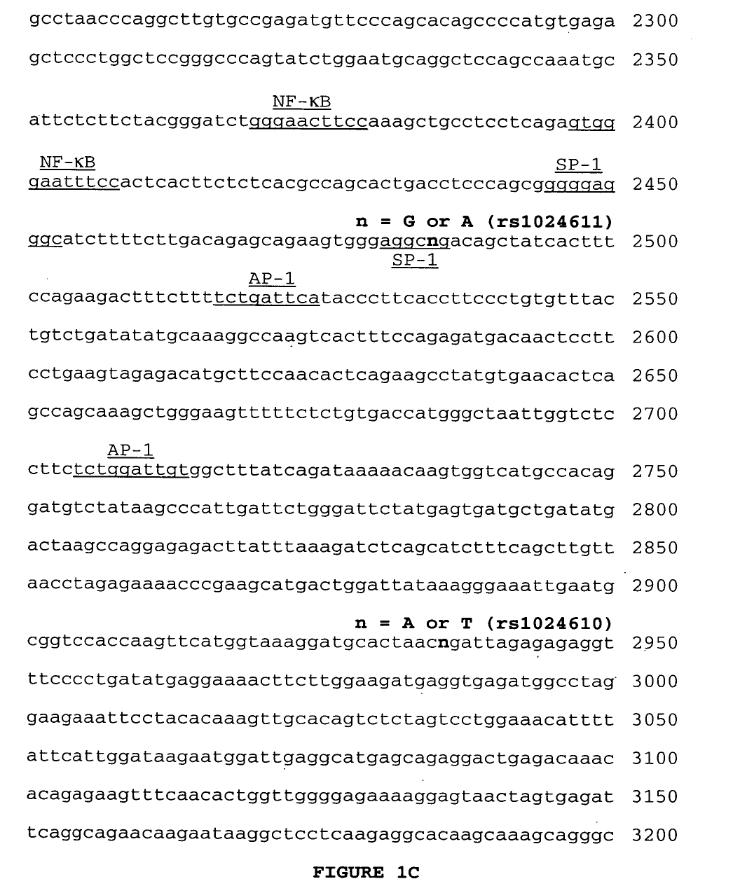

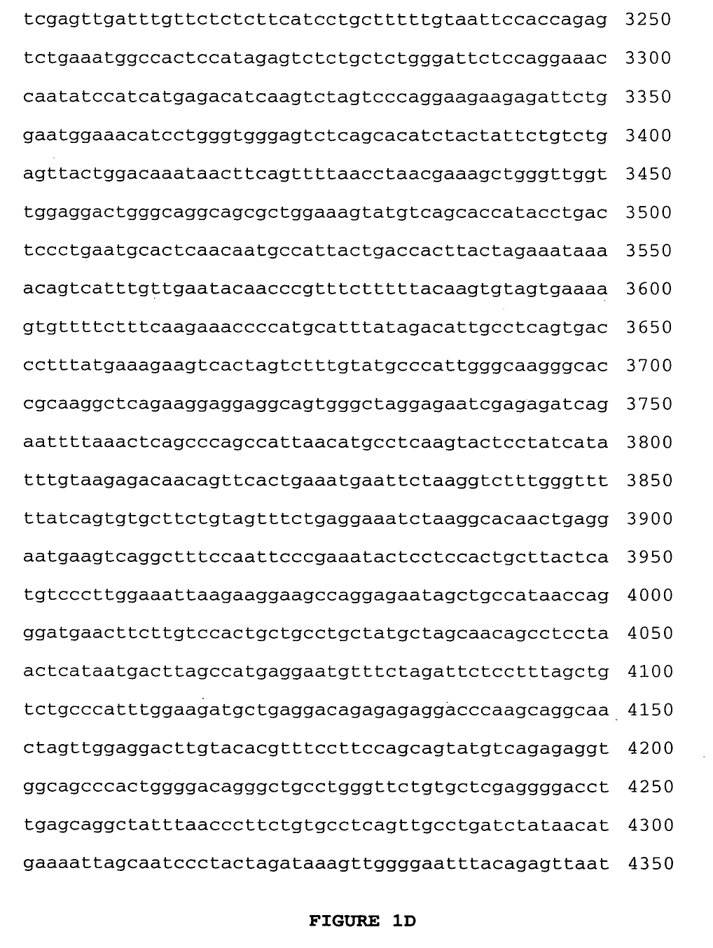

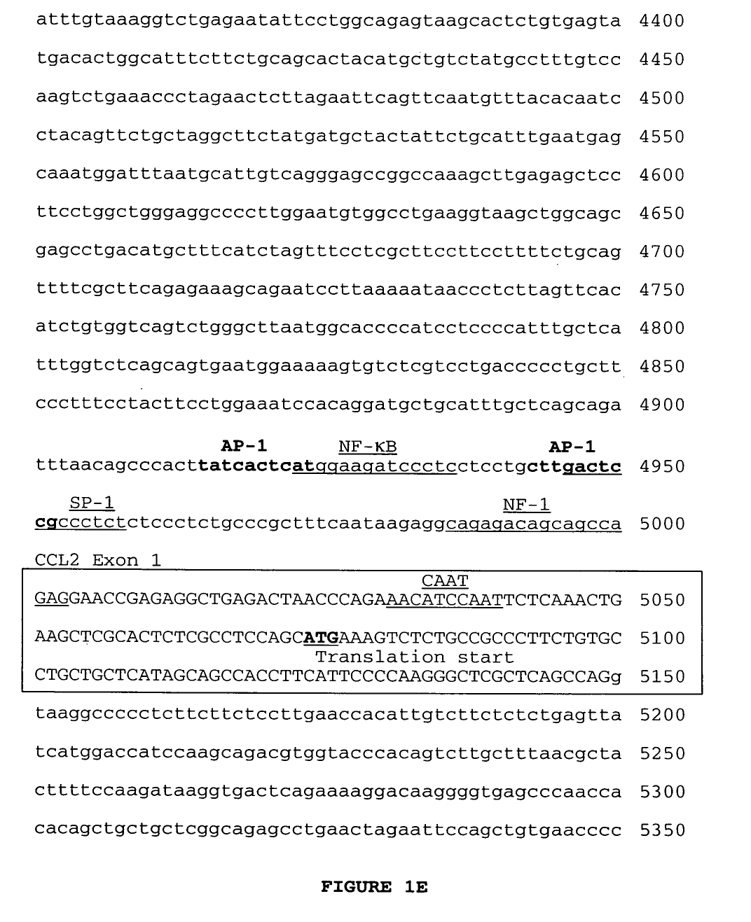

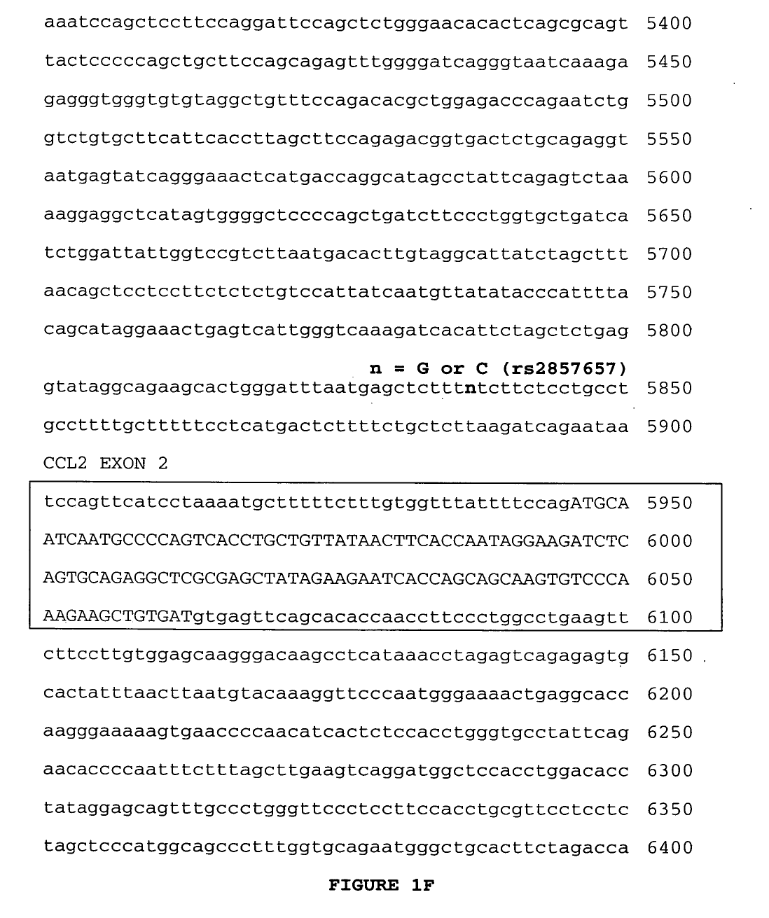

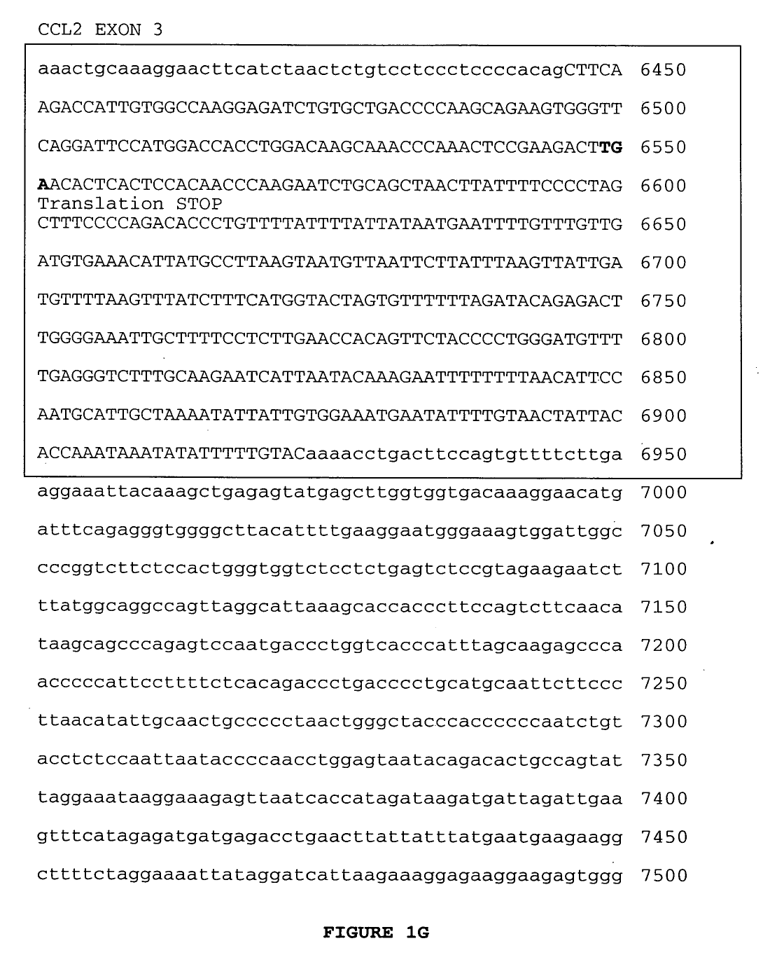

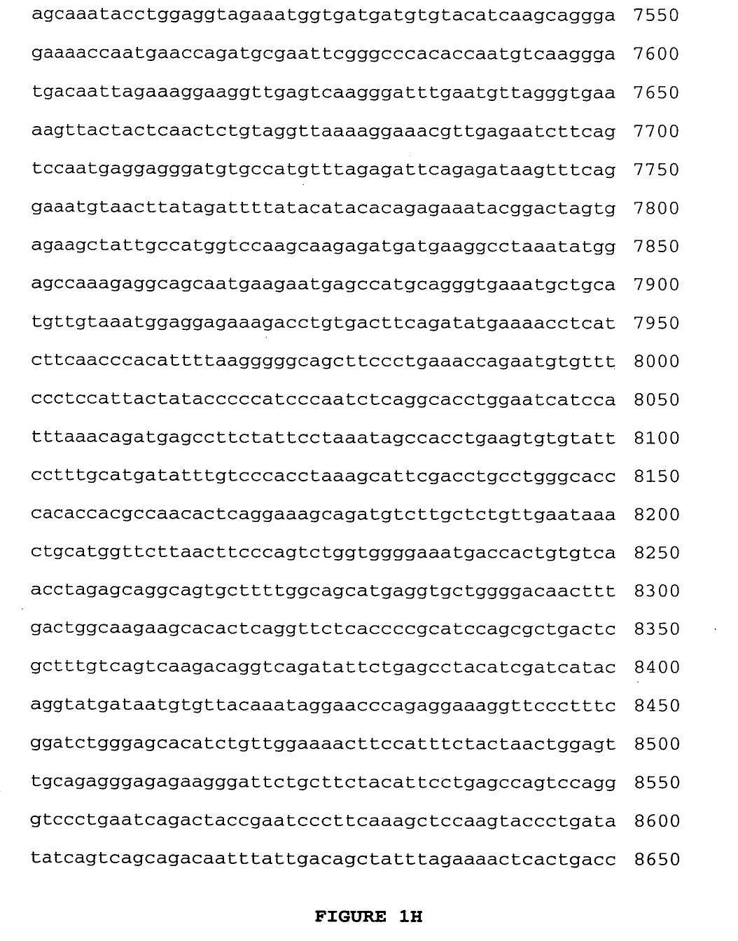

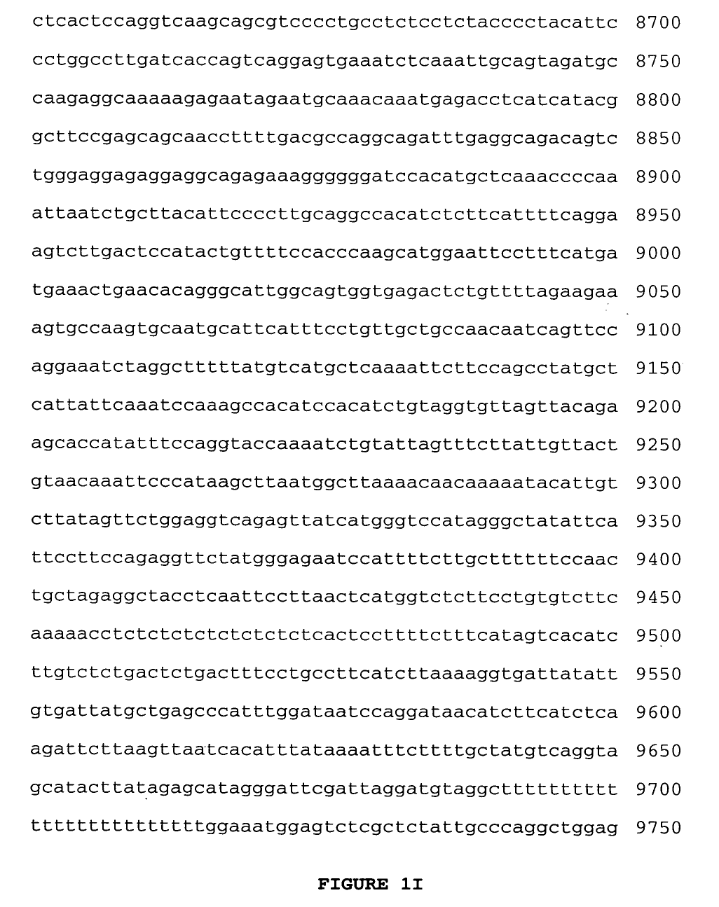

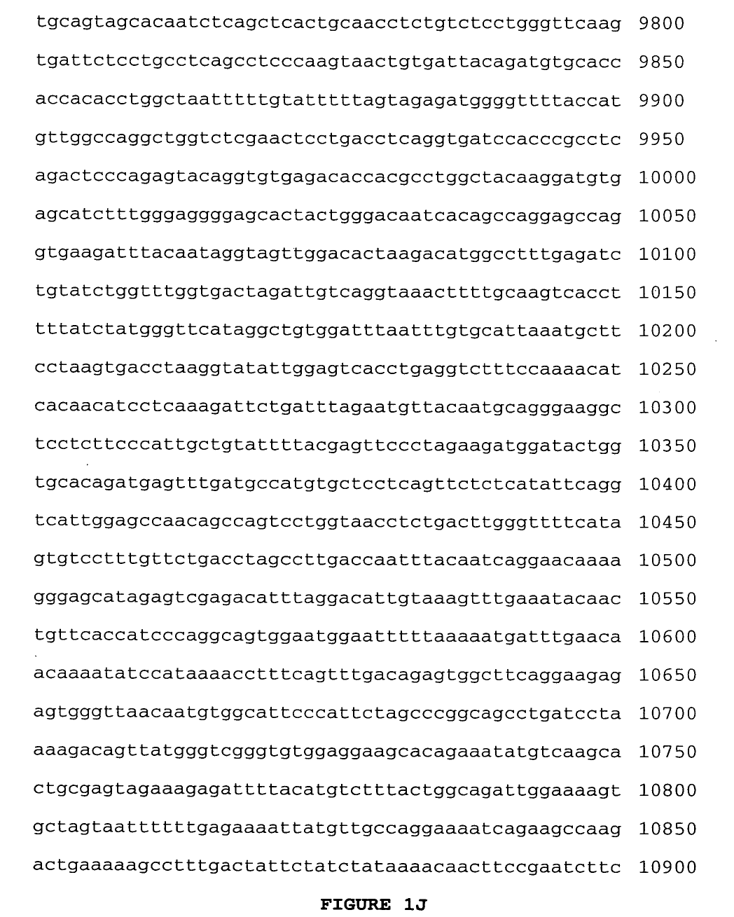

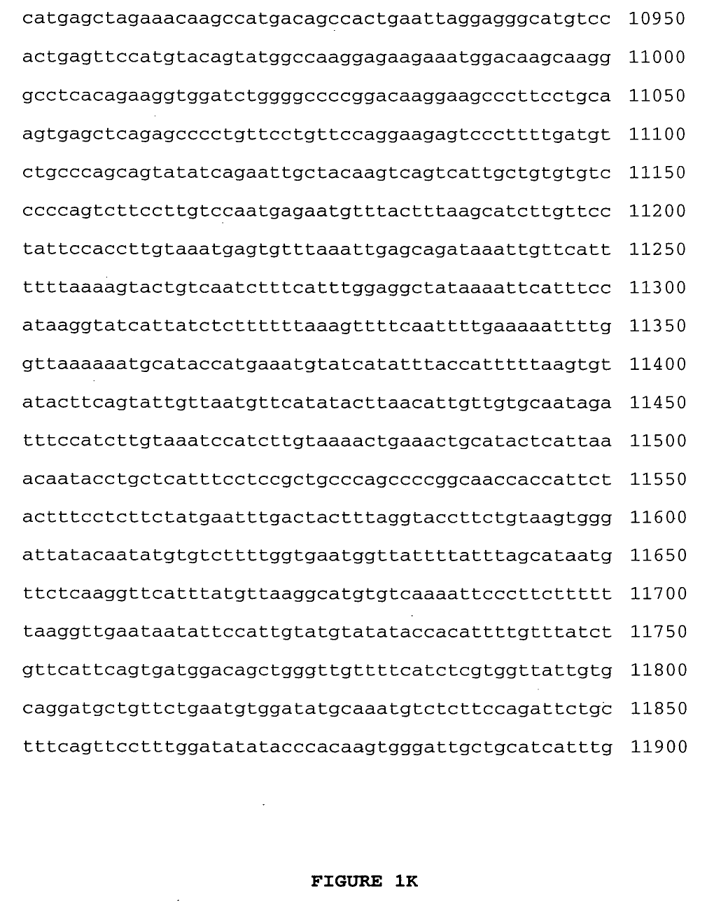

### DESCRIPTION OF THE INVENTION

Described herein are, without limitation: 1) methods and compositions for evaluating a patient having a CCR-2 mediated disorder, e.g. a CCR-2 mediated disorder as described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof; 2) methods and compositions for selecting a treatment regimen for a patient having a CCR-2 mediated disorder, e.g. a CCR-2 mediated disorder as described herein; 3) methods for selecting a patient population having a CCR-2 mediated disorder, e.g. a CCR-2 mediated disorder as described herein, for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof; 4) methods and compositions for treatment of a patient having a CCR-2 mediated disorder, e.g. a CCR-2 mediated disorder as described herein; 5) methods and compositions for monitoring the effectiveness of a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, used for the treatment of a patient having a CCR-2 mediated disorder, e.g. a CCR-2 mediated disorder as described herein; 6) methods for processing approval of payment or processing of payment for a treatment regimen of a patient having a CCR-2 mediated disorder, e.g. a CCR-2 mediated disorder as described herein; 7) arrays and kits for evaluating a patient having a CCR-2 mediated disorder, e.g. a CCR-2 mediated disorder as described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof; and 8) an article of manufacture as described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, preferred methods and materials are described herein. The content of all database accession records (e.g., representative public single nucleotide polymorphism identifier from the dbSNP database (e.g., www.ncbi.nlm.nih.gov/SNP) cited throughout this application are hereby incorporated by reference. In the case of conflict, the present specification, including definitions, will control.

Evaluation of a Patient Having a CCR-2 Mediated Disorder for Responsiveness or Non-responsiveness to a Treatment Regimen Comprising an Agent or Compound Which Inhibits the Interaction Between CCR-2 and CCL2; a CCR-2 Antagonist; a CCL2 Antagonist; or a Combination Thereof.

The methods for evaluating a patient having a CCR-2 mediated disorder, e.g., a CCR-2 mediated disorder as described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, can include, e.g., a step of determining or confirming a value for a parameter related to a patient's CCL2 expression level and/or determining or confirming a patient's genotype at a pre-selected nucleotide within the patient's CCL2 genomic sequence. In one embodiment, the method is an in vitro method. In another embodiment, the method is an in vivo method.

As used herein, the term “evaluating a patient” refers to the act of reviewing or analyzing a patient's genotype at a pre-selected nucleotide and/or a value or parameter related to a patient's expression level of a pre-selected marker. The evaluation can further include one or more of: obtaining a sample from a patient (e.g. a sample from a bodily fluid (e.g. a blood sample, a serum sample, a urine sample, a synovial fluid sample, a tear sample, a saliva sample) or a tissue sample (e.g., a skin sample or a tissue sample obtained from a biopsy) or analyzing a sample in vivo; assaying the sample or requesting an assay using the sample to obtain genomic information regarding the patient's genotype at a pre-selected nucleotide and/or to obtain a value for a parameter related to a patient's protein expression level; reviewing the patient's information using the assay results performed with the sample and/or a patient's medical records. The patient's information (e.g. genomic information or value on the patient's protein expression level) can then optionally be compared to a reference standard, e.g., publicly available information (i.e. against a reference population), to make an informed decision regarding treatment options for that patient. A “patient” as used herein is an individual, e.g., a human, having or at risk for having a disorder, e.g. a CCR-2 mediated disorder, e.g. a CCR-2 mediated disorder as described herein, who has not been treated, or has been treated with an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; a treatment regimen other than a CCR-2 antagonist treatment regimen; a treatment regimen other than a CCL2 antagonist treatment regimen; or a combination of any of the above treatment regimens.

As used herein, “CC-chemokine receptor 2” (“CCR-2”) refers to CC-chemokine receptor 2α (CCR-2α) and/or CC-chemokine receptor 2β (CCR-2β).

As used herein, a “CCR-2 mediated disorder” refers to a disorder involving the movement, e.g., recruitment from one site to another site, infiltration from one site to another site, proliferation, differentiation, and/or function of cells expressing CCR-2. Cells expressing CCR-2 include, for example, monocytes, dendritic cells, macrophages, T-cells, lymphocytes, basophils, mast cells, endothelial cells and fibroblasts. Examples of CCR-2 mediated disorders include, but are not limited to, i) inflammatory or immune disorders; ii) cardiovascular disorders; iii) proliferative disorders; iv) graft rejections; v) fibrotic diseases; vi) viral infections; vii) neurological disorders; and viii) metabolic disorders.

Inflammatory disorders and conditions can be chronic or acute. Examples of inflammatory or immune disorders include, but are not limited to, respiratory diseases such as asthma, rhinitis, hypersensitivity lung diseases, hypersensitivity pneumonitis, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); inflammatory bowel diseases, such as Crohn's disease and ulcerative colitis; spondyloarthropathies; scleroderma; psoriasis and inflammatory dermatoses such as dermatitis, eczema, atopic dermatitis, urticaria; vasculitis (e.g., necrotizing, cutaneous, and hypersensitivity vasculitis); and inflammatory glomerulopathies.

Other examples of inflammatory or immune disorders include autoimmune disorders, such as arthritis (e.g., rheumatoid arthritis, psoriatic arthritis); multiple sclerosis; systemic lupus erythematosus; myasthenia gravis; juvenile onset diabetes; nephritides such as glomerulonephritis; autoimmune thyroiditis; acquired immune deficiency syndrome (AIDS) and Behcet's disease.

Still other examples of inflammatory or immune disorders include allergic diseases and conditions, such as respiratory allergic diseases such as asthma, allergic rhinitis, hypersensitivity lung diseases, and hypersensitivity pneumonitis; anaphylaxis or hypersensitivity responses; drug allergies (e.g., to penicillin, cephalosporins); insect sting allergies; allergic contact dermatitis; vasculitis (e.g., hypersensitivity vasculitis); and histamine and IgE-mediated allergic reactions.

Cardiovascular disorders include, but are not limited to atherogenesis; atherosclerosis; coronary artery disease; myocardial infarction; stroke; acute coronary syndrome; thrombosis; peripheral vascular disease of atherosclerotic origin; hypertension; and dyslipidemia.

Proliferative disorders include, but are not limited to cancers or tumors. “Cancer” or “tumor” is intended to include any neoplastic growth in a patient, including an inititial tumor and any metastases. The cancer can be a cancer with leukocyte infiltration of the skin or organs. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkins lymphoma). Solid tumors can originate in organs, and include cancers such as lung, breast, prostate, ovary, colon, kidney, and liver. As used herein, cancer cells, including tumor cells, refer to cells that divide at an abnormal (increased) rate. Cancer cells include, but are not limited to, carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), and lymphomas (e.g., follicular lymphoma, mantle cell lymphoma, diffuse large Bcell lymphoma, malignant lymphoma, plasmocytoma, reticulum cell sarcoma, or Hodgkins disease); and tumors of the nervous system including glioma, meningoma, medulloblastoma, schwannoma or epidymoma.

Graft rejection (e.g., in transplantation) include but are not limited to allograft rejection or graft-versus-host disease.

Fibrotic diseases include, but are not limited to scleroderma.

Neurological disorders include, but are not limited to AIDS related dementia and pain (e.g. neuropathic pain).

Metabolic disorders include, but are not limited to diabetes.

Other diseases or conditions (including CCR-2-mediated diseases or conditions) in which undesirable inflammatory responses are to be inhibited, include, but are not limited to, reperfusion injury; certain hematologic malignancies; cytokine-induced toxicity (e.g., septic shock, endotoxic shock); polymyositis; dermatomyositis; and granulomatous diseases including sarcoidosis.

An individual's genotype at a pre-selected nucleotide or allele within the genomic sequence of CCL2 may be indicative of both responsive and non-responsive patients to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. In one embodiment, the pre-selected nucleotide or allele is present within a non-coding region (e.g. within an intron or within a regulatory region) of CCL2. The term “regulatory region” as used herein refers to a region of a patient's CCL2 genomic sequence occurring 5′ to the transcription start site and comprising regulatory elements, such as AP-1 sites, NF-kB sites, SP-1 sites, NF-1 sites and CAAT sites as described herein. An “intron” as used herein refers to non-coding regions within the genomic sequence of CCL2 found 3′ to the first nucleic acid of the transcription start site. Examples of introns within a CCL2 genomic sequence include nucleotides at positions 5150 to 5945, as numbered in SEQ ID NO:1, and nucleotides at positions 6064 to 6445, as numbered in SEQ ID NO:1. In one embodiment, the pre-selected nucleotide or allele is nucleotide 2485 as numbered in SEQ ID NO:1, or a nucleotide or allele in linkage disequilibrium with the nucleotide at position 2485, for example, but not limited to, nucleotides at positions 2236, 2936 and 5837 as numbered in SEQ ID NO:1. For example, homozygosity or heterozygosity for the G allele at position 2485 as numbered in SEQ ID NO:1, or a nucleotide or allele in linkage disequilibrium with the nucleotide or allele at position 2485, is indicative of a responsive patient to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. Homozygosity for the A allele at position 2485, as numbered in SEQ ID NO:1, or a nucleotide or allele in linkage disequilibrium with the nucleotide or allele at position 2485, may be indicative of a non-responsive patient to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof.

A patient having a CCR-2 mediated disorder is “responsive” to a therapeutic agent (e.g., CCR-2 antagonist or CCL2 antagonist) or to a treatment regimen (e.g., a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof) if, for example, the inflammation rate or growth rate of the affected area, e.g., atherosclerotic plaque, tumor, sites of inflammation, fibrotic lesions, etc. is slowed or inhibited as a result of contact with the therapeutic agent, or as a result of treatment with the treatment regimen, compared to the growth rate or rate of inflammation in the absence of contact with the therapeutic agent, or in the absence of treatment with the treatment regimen. Inflammation rate can be measured in a variety of ways, for instance, by measuring expression of inflammation markers appropriate for the CCR-2 mediated disorder, for example by measuring the level of C Reactive Protein (CRP) in patients having atherosclerosis. Inflammation rate can also be measured, for example, by using Positron Emission Tomography (PET) in, for example, patients having inflammation to the lungs, or by using Magnetic Resonance Imaging (MRI) in, for example, patients having multiple sclerosis. Inflammation rate can also be measured, for example, by enumerating swollen and tender joints in the hands and limbs (e.g. wrists, knees, hips) for patients having rheumatoid arthritis. Growth of a cancer can be measured in a variety of ways, for instance, the size of a tumor or the expression of tumor markers appropriate for that tumor type may be measured. Still further, measures of responsiveness can be assessed using additional criteria beyond growth rates or inflammation rates, including patient quality of life, less dependence on non-CCR-2 antagonists or non-CCL2 antagonists, such as inhalers, insulin, anti-inflammatory drugs, etc., degree of inflammation, degree of metastases, etc. Clinical prognostic markers and variables can be assessed (e.g., CRP in atherosclerosis) in applicable situations.

A patient having a CCR-2 mediated disorder is “non-responsive” to a therapeutic agent (e.g. CCR-2 antagonist or CCL2 antagonist) or to a treatment regimen (e.g., a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof) if, for example, the inflammation rate or growth rate of the affected area, e.g., atherosclerotic plaque, tumor, sites of inflammation, fibrotic lesions, etc. is slowed or inhibited as a result of contact with the therapeutic agent, or as a result of treatment with the treatment regimen, compared to the growth rate or rate of inflammation in the absence of contact with the therapeutic agent, or in the absence of treatment with the treatment regimen. As stated above, inflammation rate can be measured in a variety of ways, for instance, by measuring expression of inflammation markers appropriate for the CCR-2 mediated disorder, for example by measuring the level of CRP in patients having atherosclerosis. Inflammation rate can also be measured, for example, by using PET in, for example, patients having inflammation to the lungs, or by using MRI in, for example, patients having multiple sclerosis. Inflammation rate can also be measured, for example, by enumerating swollen and tender joints in the hands and limbs (e.g. wrists, knees, hips) for patients having rheumatoid arthritis. Growth of a cancer can be measured in a variety of ways, for instance, the size of a tumor or the expression of tumor markers appropriate for that tumor type may be measured. The quality of being non-responsive to a therapeutic agent is a highly variable one, with different CCR-2 mediated disorders exhibiting different levels of “non-responsiveness” to a given therapeutic agent, under different conditions. Still further, measures of non-responsiveness can be assessed using additional criteria beyond growth rates or inflammation rates, including patient quality of life, continued or increased dependence on non-CCR-2 antagonists or non-CCL2 antagonists, such as inhalers, insulin, anti-inflammatory drugs, etc., degree of inflammation, degree of metastases, etc. Clinical prognostic markers and variables can be assessed (e.g., CRP in atherosclerosis) in applicable situations.

“Linkage Disequilibrium” as used herein refers to a non-random association of a first chromosomal region, for example, a Single Nucleotide Polymorphism (SNP), with a second chromosomal region, for example, a SNP. In a preferred embodiment, the method includes determining or reviewing a nucleotide in linkage disequilibrium with the nucleotide at position 2485 as numbered in SEQ ID NO:1 (rs1024611). Techniques for determining whether any two polymorphic sites are in linkage disequilibrium are well-known in the art (Weir B. S. 1996 Genetic Data Analysis II, Sinauer Associates, Inc. Publishers, Sunderland, Mass.). A non-random association of a chromosomal region (e.g. a SNP) in linkage disequilibrium corresponds to an r² value greater than 0.5. In a preferred embodiment, the r² value is greater than 0.6, 0.7, 0.8 or 0.9 (where r² of 1.0=perfect linkage disequilibrium (i.e. perfect correlation between SNPs). (See e.g., Carlson C. S. et al. 2004 Am. J. Hum. Genet. 74:106-120, which is incorporated herein by reference).

As used herein, a “Single Nucleotide Polymorphism (SNP)” is the specific pair of nucleotides observed at a single polymorphic site. In rare cases, three or four nucleotides may be found at the site.

As used herein, a “polymorphism” is the sequence variation observed in an individual at a polymorphic site. Polymorphisms include nucleotide substitutions, insertions, deletions and microsatellites and may, but need not, result in detectable differences in gene expression or protein function. As used herein, a “polymorphic site” is a position within a locus at which at least two alternative sequences are found in a population, the most frequent of which has a frequency of no more than 99%. A “locus”, as used herein, is a location on a chromosome or DNA molecule corresponding to a gene or a physical or phenotypic feature.

A “polymorphic variant” is a gene, mRNA, cDNA, polypeptide or peptide whose nucleotide or amino acid sequence varies from a reference sequence due to the presence of a polymorphism in the gene.

As used herein, a “CCR-2 antagonist” is a molecule which binds CCR-2 and inhibits the interaction of CCR-2 with a ligand e.g., inhibits the binding of CCR-2 to a ligand (e.g., CCL2 (MCP-1), CCL7 (MCP-3), CCL8 (MCP-2), CCL13 (MCP-4) or CCL16 (HCC4)) or inhibits signal transduction induced by binding of CCR-2 to a ligand (e.g., inhibits CCR-2 binding to a cytoplasmic G-protein). Examples of CCR-2 antagonists include peptides, polypeptides (e.g., soluble fragments of ligands, non-naturally occurring peptides that bind CCR-2 (e.g., peptides obtained by phage display)), antibodies or fragments thereof, and non-protein molecules (e.g. small molecules). Examples of antibodies and non-protein molecules which are CCR-2 antagonists include the molecules described in the patent applications listed in Tables III, the contents of which are incorporated herein by reference.

As used herein, a “CCL2 antagonist” is a molecule which binds CCL2 and inhibits a function of CCL2 (e.g., inhibits the binding of CCL2 to a binding partner, e.g., a chemokine receptor, e.g., CCR-2) or inhibits signal transduction induced by binding of CCL2 to a chemokine receptor (e.g., CCR-2) (e.g., inhibits CCR-2 binding to a cytoplasmic G-protein). Examples of CCL2 antagonists include peptides, polypeptides (e.g., soluble fragments of binding partners, e.g., CCR-2, non-naturally occurring peptides that bind CCL2 (e.g., peptides obtained by phage display)), antibodies or fragments thereof, and non-protein molecules (e.g., small molecules). Examples of antibodies and non-protein molecules which are CCL2 antagonists include the molecules described in the patent applications listed in Tables IV, the contents of which are incorporated herein by reference.

“Treatment regimen” as used herein, refers to treatment with a molecule alone, or in combination with another molecule. A treatment regimen also refers to dose amount, the frequency of dosing and the number of times a molecule, or combination of molecules, is administered.

The term “CCR-2 antagonist treatment regimen” as used herein, refers to a treatment with a CCR-2 antagonist alone or in combination with one or more additional agents. A CCR-2 antagonist treatment regimen also refers to dose amounts, the frequency of dosing and the number of times a CCR-2 antagonist is administered. The terms “dosing schedule” or “administration schedule” as used herein refer to both the frequency of dosing and the number of times the CCR-2 antagonist is administered.

The term “CCL2 antagonist treatment regimen” as used herein, refers to treatment with a CCL2 antagonist alone or in combination with one or more additional agents. A CCL2 antagonist treatment regimen also refers to dose amounts, the frequency of dosing and the number of times a CCL2 antagonist is administered. The terms “dosing schedule” or “administration schedule” as used herein refer to both the frequency of dosing and the number of times the CCL2 antagonist is administered.

An “agent or compound which inhibits the interaction between CCR-2 and CCL2”, as used herein, is a molecule which prevents the association (e.g., binding) between CCR-2 and CCL2. In one embodiment, such molecules may exert their effect by binding one or both of the target molecules (e.g., CCR-2 or CCL2) and preventing the molecules from directly binding or preventing CCR-2 from signaling (e.g., signal transduction through the binding of CCR-2 to cytoplasmic G proteins). In another embodiment, such molecules may exert their effect without binding to either CCR-2 or CCL2, by, for example, binding to a transcription factor responsible for the protein expression of CCR-2 or CCL2, thereby preventing expression of either CCR-2 or CCL2. In another embodiment, such molecules may exert their activity by sequestering CCR-2 or CCL2 without inducing signal transduction.

In one embodiment of the invention, a patient is selected for evaluation based upon the patient having one or more symptoms related to a CCR-2 mediated disorder, e.g., as described herein, or one or more other indicator(s) of propensity for the CCR-2 mediated disorder, and then determining, recommending or selecting an appropriate treatment regimen, or beginning, continuing, discontinuing, changing or altering the treatment regimen.

### Determining a Value for a Parameter Related to a Patient's CCL2 Expression

As used herein, the term “value for a parameter related to a patient's CCL2 expression” refers to a comparable figure, e.g., a number, e.g., a normalized number, which can be used to compare one individual's CCL2 expression level to another individual's CCL2 expression level.

In certain aspects, determining or confirming a value for a parameter related to a patient's CCL2 expression comprises detection of mRNA. Such detection can be carried out by any relevant method, including e.g., PCR, northern, nucleotide array detection, in vivo imaging using probes capable of detection of the appropriate nucleic acid. In other aspects, determining a value for a parameter related to a patient's CCL2 expression comprises detection of protein. Such detection can be carried out using any relevant method for protein detection, including e.g., ELISA, western blot, immunoassay, protein array detection, in vivo imaging using probes capable of detection of the appropriate peptide.

An exemplary method for detecting the presence or absence of a nucleic acid or polypeptide corresponding to a marker of the invention (e.g., CCL2) in a biological sample involves obtaining a biological sample (e.g. a biological sample as described herein) from a patient having a CCR-2 mediated disorder and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations, In Situ Hybridizations (ISH) and TaqMan assays (Applied Biosystems). In vitro techniques for detection of a polypeptide corresponding to a marker of the invention (e.g., CCL2) include Enzyme Linked Immunosorbent Assays (ELISAs), Western blots, immunoprecipitations and immunofluorescence. In vitro techniques for detection of genomic DNA include Southern hybridizations. Furthermore, in vivo techniques for detection of a polypeptide corresponding to a marker of the invention (e.g., CCL2) include introducing into a subject a labeled antibody directed against the polypeptide. For example, the antibody can be labeled with a radioactive agent whose presence and location in a subject can be detected by standard imaging techniques.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay involves anchoring the marker or probe onto a solid phase support, also referred to as a substrate, and detecting target marker/probe complexes anchored on the solid phase at the end of the reaction. In one example of such a method, a sample from a subject, which is to be assayed for presence and/or concentration of marker, can be anchored onto a carrier or solid phase support. In another example, the reverse situation is possible, in which the probe can be anchored to a solid phase and a sample from a subject can be allowed to react as an unanchored component of the assay. One example of such an example includes use of an array or chip which contains a predictive marker or marker set anchored for expression analysis of the sample.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain aspects, the surfaces with immobilized assay components can be prepared in advance and stored. Other suitable carriers or solid phase supports for such assays include any material capable of binding the class of molecule to which the marker or probe belongs. Well-known supports or carriers include, but are not limited to, glass, polystyrene, nylon, polypropylene, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein. In one example, when the probe is the unanchored assay component, it can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of Fluorescence Energy Transfer (FET) (see, for example, Lakowicz et al., U.S. Pat. No. 5,631,169; Stavrianopoulos, et al., U.S. Pat. No. 4,868,103). A fluorophore label on the first ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another example, determination of the ability of a probe to recognize a marker can be accomplished without labeling either assay component (probe or marker) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another example, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6): 141-8; Hage, D. S., and Tweed, S. A. J Chronatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

The level of mRNA corresponding to the marker can be determined both by in situ and by in vitro formats in a biological sample (e.g., biological sample as described herein) using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

One diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 but not more than 2000, 1000, 750, 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array or other chip array described herein. A skilled artisan can readily adapt known mRNA detection methods for use in detecting the level of mRNA encoded by the markers of the present invention.

An alternative method for determining the level of mRNA corresponding to a marker of the present invention in a sample involves the process of nucleic acid amplification, e.g., by rtPCR (the experimental description set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad Sci. USA 87:1874-1878), transcriptional amplification system (Kwoh et al., 1989, Proc. Natl. Acad Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of interest in a gene (plus and minus strands, respectively, or vice-versa) and contain a short region in between. In general, amplification primers are from about 10 to 30 nucleotides in length and flank a region, e.g., from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a reference gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample from a patient having a CCR-2 mediated disorder, to another sample, e.g., another patient having a CCR-2 mediated disorder or a patient sample from a patient who does not have a CCR-2 mediated disorder, or between samples from different sources.

In another aspect of the present invention, a polypeptide corresponding to a marker is detected. A preferred agent for detecting a polypeptide of the invention is an antibody capable of binding to a polypeptide corresponding to a marker of the invention, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

A variety of formats can be employed to determine whether a sample contains a protein that binds to a given antibody. Examples of such formats include, but are not limited to, enzyme immunoassay (EIA), radioimmunoassay (RIA), Western blot analysis and ELISA. A skilled artisan can readily adapt known protein/antibody detection methods for use in determining whether a patient sample expresses a marker of the present invention.

In one format, antibodies, or antibody fragments, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from a patient's biological sample, (e.g., a biological sample as described herein), can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

Another method for determining the level of a polypeptide corresponding to a marker is mass spectrometry. For example, intact proteins or peptides, e.g., tryptic peptides can be analyzed from a sample, e.g., blood, plasma, urine, etc, containing one or more polypeptide markers. The method can further include treating the sample to lower the amounts of abundant proteins, e.g. serum albumin, to increase the sensitivity of the method. For example, liquid chromatography can be used to fractionate the sample so portions of the sample can be analyzed separately by mass spectrometry. The steps can be performed in separate systems or in a combined liquid chromatography/mass spectrometry system (LC/MS, see for example, Liao, et al. Arthritis Rheum. 50:3792-3803 (2004)). The mass spectrometry system also can be in tandem (MS/MS) mode. The charge state distribution of the protein or peptide mixture can be acquired over one or multiple scans and analyzed by statistical methods, e.g. using the retention time and mass-to-charge ratio (m/z) in the LC/MS system, to identify proteins expressed at statistically significant levels differentially in samples from patients responsive or non-responsive to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. Examples of mass spectrometers which can be used are an ion trap system (ThermoFinnigan, San Jose, Calif.) or a quadrupole time-of-flight mass spectrometer (Applied Biosystems, Foster City, Calif.). The method can further include the step of peptide mass fingerprinting, e.g. in a matrix-assisted laser desorption ionization with time-of-flight (MALDI-TOF) mass spectrometry method. The method can further include the step of sequencing one or more of the tryptic peptides. Results of this method can be used to identify proteins from primary sequence databases, e.g. maintained by the National Center for Biotechnology Information, Bethesda, Md., or the Swiss Institute for Bioinformatics, Geneva, Switzerland, and based on mass spectrometry tryptic peptide m/z base peaks.

In one embodiment, the expression level of a marker in an individual can be one of the individual's phenotypes. As used herein, “phenotype” refers to a physical characteristic or property of an individual as determined by the individual's genotype. A “genotype” is the sequence of nucleotide pair(s) found at one or more polymorphic sites in a locus on a pair of homologous chromosomes in an individual. For example, expression of CCL2 in an individual can be correlated to the individual's genotype of the CCL2 genomic sequence at a pre-selected nucleotide or allele, or at one or more nucleotides or alleles in linkage disequilibrium with the pre-selected nucleotide or allele. In one embodiment, the pre-selected nucleotide or allele is present within a non-coding region (e.g., an intron or a regulatory region) of the CCL2 genomic sequence. In another embodiment, the pre-selected nucleotide or allele is a nucleotide at position 2485 as numbered in SEQ ID NO:1, or one or more nucleotides or alleles in linkage disequilibrium with the nucleotide at position 2485 as numbered in SEQ ID NO:1, e.g., nucleotides at positions 2236, 2936 and 5837 as numbered in SEQ ID NO:1. In one embodiment, CCL2 expression levels are greater in individuals who carry a G allele at position 2485 as numbered in SEQ ID NO:1, than individuals who carry a homozygous A allele at position 2485 as numbered in SEQ ID NO:1. In another embodiment, individuals who are homozygous for the G allele at position 2485 as numbered in SEQ ID NO:1 have greater CCL2 expression levels than individuals who are heterozygous at position 2485 (e.g., individuals who carry a G allele and an A allele), and in turn, heterozygous individuals have greater CCL2 expression levels than individuals who are homozygous for the A allele at position 2485.

An “allele” is a particular form of a genetic locus, distinguished from other forms by its particular nucleotide sequence.

In one embodiment, the expression level can be provided as a relative expression level. To determine a relative expression level of a marker, the level of expression of the marker is determined for 10 or more samples from patients having the same genotype, preferably 50 or more samples, e.g., up to about 100, 500, 1000, 1500, 2000 samples, prior to the determination of the expression level for the sample in question. The mean expression level of each of the markers assayed in the larger number of samples is determined and this is used as a baseline expression level for the marker. The expression level of the marker determined for the test sample (absolute level of expression) is then divided by the mean expression value obtained for that marker. This provides a relative expression level.

In one embodiment, the value for a patient is compared to a reference standard for CCL2 expression. In one embodiment, the reference standard is correlated with the genotype of the nucleic acid at position 2485 as numbered in SEQ ID NO:1, or at one or more nucleotides or alleles in linkage disequilibrium with a nucleic acid at position 2485. For example, the reference standard can comprise three different values, for example, it can be provided for individuals who are i) homozygous for the A allele at position 2485 as numbered in SEQ ID NO:1; ii) homozygous for the G allele at position 2485 as numbered in SEQ ID NO:1; and iii) heterozygous (e.g. individuals who carry an A allele and a G allele) at position 2485 as numbered in SEQ ID NO:1.

### Determining or Confirming a Patient's Genotype at a Pre-selected Nucleotide or Allele Within the Patient's CCL2 Genomic Sequence

The identification of a correlation between a genotype at, for example, a pre-selected nucleotide or allele within the CCL2 genomic sequence, and clinical responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, can be the basis for designing a diagnostic method to predict those individuals who will or will not respond to a treatment regimen, e.g., a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. Alternatively, such methods can also be used to predict individuals who will respond or will not respond to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, as described herein, at varying degrees, thus identifying individuals who may require more treatment, i.e., a greater dose of a drug, or less treatment, i.e., a smaller dose of a drug. Such methods can also be used to predict individuals who will respond to a treatment regimen other than a CCR-2 antagonist treatment regimen or other than a CCL2 antagonist treatment regimen. The diagnostic method for determining or confirming a patient's genotype may be, for example, a direct DNA test (i.e., genotyping or haplotyping one or more of the polymorphic sites in the CCL2 gene), a serological test, or a physical exam measurement. The term “genotyping”, as used herein, refers to a process for determining a genotype of an individual. The term “haplotyping”, as used herein, refers to a process for determining one or more haplotypes in an individual and includes use of family pedigrees, molecular techniques and/or statistical inference. A “haplotype”, as used herein, is the ordered combination of polymorphisms in the sequence of each form of a gene that exists in the population. In one embodiment, the method is an in vitro method. In another embodiment, the method is an in vivo method

In one embodiment, the invention provides compositions and methods for genotyping an individual's CCL2 genomic sequence. The compositions comprise at least one CCL2 genotyping oligonucleotide. In one embodiment, a CCL2 genotyping oligonucleotide is a probe or primer capable of hybridizing to a target region that is located close to, or that contains, a pre-selected nucleotide within the CCL2 genomic sequence. In one embodiment, a CCL2 genotyping oligonucleotide is a probe or primer capable of hybridizing to a target region that is located close to, or that contains, the nucleotide located at position 2236 of the CCL2 gene, as numbered in SEQ ID NO:1. In another embodiment, a CCL2 genotyping oligonucleotide is a probe or primer capable of hybridizing to a target region that is located close to, or that contains, the nucleotide located at position 2485 of the CCL2 gene, as numbered in SEQ ID NO:1. In another embodiment, a CCL2 genotyping oligonucleotide is a probe or primer capable of hybridizing to a target region that is located close to, or that contains, the nucleotide located at position 2936 of the CCL2 gene, as numbered in SEQ ID NO:1. In yet another embodiment, a CCL2 genotyping oligonucleotide is a probe or primer capable of hybridizing to a target region that is located close to, or that contains, the nucleotide located at position 5837 of the CCL2 gene, as numbered in SEQ ID NO:1. In yet another embodiment, a CCL2 genotyping oligonucleotide is a probe or primer capable of hybridizing to a target region that is located close to, or that contains, a nucleotide in linkage disequilibrium with any of the above identified polymorphic sites within the CCL2 gene.

The term “probe or primer” refers to any molecule which is capable of selectively binding to a specifically intended target molecule, for example a polymorphic site or locus of a SNP of the invention. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic monomers.

As used herein, the term “oligonucleotide” refers to a polynucleotide molecule having less than about 100 nucleotides. In another embodiment, the oligonucleotide of the invention is 10 to 35 nucleotides long. In yet another embodiment, the oligonucleotide is between 15 and 30 nucleotides long. In another embodiment, the oligonucleotide is between 20 and 25 nucleotides in length. The exact length of the oligonucleotide will depend on many factors that are routinely considered and practiced by the skilled artisan. Oligonucleotides of the invention may be prepared by chemical synthesis using any suitable methodology known in the art, or may be derived from a biological sample, for example, by restriction digestion. The oligonucleotides may be labeled, according to any technique known in the art, including use of radiolabels, fluorescent labels, enzymatic labels, proteins, haptens, antibodies, sequence tags and the like.

Genotyping oligonucleotides of the invention must be capable of specifically hybridizing to a target region of a CCL2 polynucleotide. As used herein, specific hybridization means the oligonucleotide forms an anti-parallel double-stranded structure with the target region under certain hybridizing conditions, while failing to form such a structure when incubated with a non-target region or a non-CCL2 polynucleotide under the same hybridizing conditions. Preferably, the oligonucleotide specifically hybridizes to the target region under conventional high stringency conditions. The skilled artisan can readily design and test oligonucleotide probes and primers suitable for detecting polymorphisms in the CCL2 gene using known sequence information for the CCL2 gene and routine techniques. Conventional hybridization conditions are described, for example, by Sambrook J. et al., in Molecular Cloning, A Laboratory Manual, 2.sup.nd Edition, Cold Spring Harbor Press, Cold Spring Harbor, N.Y. (1989) and by Haymes, B. D. et al. in Nucleic Acid Hybridization, A Practical Approach, IRL Press, Washington, D.C. (1985).

A nucleic acid molecule such as an oligonucleotide or polynucleotide is said to be a “perfect” or “complete” complement of another nucleic acid molecule if every nucleotide of one of the molecules is complementary to the nucleotide at the corresponding position of the other molecule. “Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion. While perfectly complementary oligonucleotides are preferred for detecting polymorphisms, departures from complete complementarity are contemplated where such departures do not prevent the molecule from specifically hybridizing to the target region. For example, an oligonucleotide primer may have a non-complementary fragment at its 5′ end, with the remainder of the primer being complementary to the target region. Alternatively, non-complementary nucleotides may be interspersed into the oligonucleotide probe or primer as long as the resulting probe or primer is still capable of specifically hybridizing to the target region.

As used herein, the term “hybridizes under stringent conditions” is intended to describe conditions for hybridization and washing under which nucleotide sequences at least 30%, 40%, 50%, or 60% homologous to each other typically remain hybridized to each other. The conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A non-limiting example of stringent hybridization conditions are hybridization in 6× sodium chloride/sodium citrate (SSC) at about 45° C., followed by one or more washes in 0.2×SSC, 0.1% SDS at 50-65° C. Another example of stringent hybridization conditions is hybridization in 0.5M sodium phophate, 7% SDS at 65° C., followed by one or more washes in 0.2×SSC at 65° C.

Preferred genotyping oligonucleotides of the invention are allele-specific oligonucleotides. As used herein, the term allele-specific oligonucleotide (ASO) means an oligonucleotide that is able, under sufficiently stringent conditions, to hybridize specifically to one allele of a gene, or other locus, at a target region containing a polymorphic site while not hybridizing to the corresponding region in another allele(s). As understood by the skilled artisan, allele-specificity will depend upon a variety of readily optimized stringency conditions, including salt and formamide concentrations, as well as temperatures for both the hybridization and washing steps. Examples of hybridization and washing conditions typically used for ASO probes are found in Kogan et al., “Genetic Prediction of Hemophilia A” in PCR Protocols, A Guide to Methods and Applications, Academic Press, 1990 and Ruano et al., 87 Proc. Natl. Acad. Sci. USA 6296-6300, 1990. Typically, an ASO will be perfectly complementary to one allele while containing a single mismatch for another allele.

Allele-specific oligonucleotides of the invention include ASO probes and ASO primers. ASO probes which usually provide good discrimination between different alleles are those in which a central position of the oligonucleotide probe aligns with the polymorphic site in the target region. An ASO primer of the invention has a 3′ terminal nucleotide, or preferably a 3′ penultimate nucleotide, that is complementary to only one nucleotide of a particular SNP, thereby acting as a primer for polymerase-mediated extension only if the allele containing that nucleotide is present. ASO probes and primers hybridizing to either the coding or noncoding strand are contemplated by the invention.

Other genotyping oligonucleotides of the invention hybridize to a target region located one to several nucleotides downstream of one of the polymorphic sites identified herein. Such oligonucleotides are useful in polymerase-mediated primer extension methods for detecting one of the polymorphisms described herein and therefore such genotyping oligonucleotides are referred to herein as “primer-extension oligonucleotides”. In a preferred embodiment, the 3′-terminus of a primer-extension oligonucleotide is a deoxynucleotide complementary to the nucleotide located immediately adjacent to the polymorphic site.

In some embodiments, a composition contains two or more differently labeled genotyping oligonucleotides for simultaneously probing the identity of nucleotides at two or more polymorphic sites. It is also contemplated that primer compositions may contain two or more sets of allele-specific primer pairs to allow simultaneous targeting and amplification of two or more regions containing a polymorphic site.

CCL2 genotyping oligonucleotides of the invention may also be immobilized on or synthesized on a solid surface such as a microchip, bead, or glass slide. Such immobilized genotyping oligonucleotides may be used in a variety of polymorphism detection assays, including but not limited to probe hybridization and polymerase extension assays. Immobilized CCL2 genotyping oligonucleotides of the invention may comprise an ordered array of oligonucleotides designed to rapidly screen a DNA sample for polymorphisms in multiple genes at the same time.

In one embodiment, an individual's genotype at position 2485 as numbered in SEQ ID NO:1 may be determined using Applied Biosystems' TaqMan® SNP Genotyping Assay (assay ID C_{—}2590362_{—}10).

In another embodiment, an individual's genotype may be determined using probes and primers, e.g., probes and primers listed in Table I.

TABLE I

| Assay | Position 2236 | Position 2485 | Position 2936 | Position 5837 |
|---|---|---|---|---|
| reagents | (rs2857654) | (rs1024611) | (rs1024610) | (rs2857657) |
| Forward | TGACCCTCATT | CACAGGGAAGG | GCTCATGCCTC | TAGGCAGAAGC |
| amplification | TTCCCCATA | TGAAGGGTA | AATCCATTC | ACTGGGATT |
| primer | (SEQ ID NO: 10) | (SEQ ID NO: 11) | (SEQ ID NO: 12) | (SEQ ID NO: 13) |
| Reverse | TGGAAATTCCC | AAAGCTGCCTC | AGAGAAAACCC | GCATTGATTGC |
| amplification | ACTCTGAGG | CTCAGAGTG | GAAGCATGA | ATCTGGAAA |
| primer | (SEQ ID NO: 14) | (SEQ ID NO: 15) | (SEQ ID NO: 16) | (SEQ ID NO: 17) |
| SBE probe | CCAAGCGAGAG | GTCTTCTGGAA | GAAACCTCTCT | GATTTAATGAG |
| | TCCAACC | AGTGA | CTAATC | CTCTTT |
| | (SEQ ID NO: 18) | (SEQ ID NO: 19) | (SEQ ID NO: 20) | (SEQ ID NO: 21) |
| SBE probe | ddA | ddC | ddA | ddC |
| assay | ddC | ddT | ddT | ddG |
| nucleotides | | | | |
| Hybridization | TCCAACCAAGG | GAAAGTGATAG | TCTCTAATCAG | GCTCTTTCTCT |
| probe 1 | TTTGT | CTGTC | TTAGTGCA | TCTC |
| | (SEQ ID NO: 22) | (SEQ ID NO: 23) | (SEQ ID NO: 24) | (SEQ ID NO: 25) |
| Hybridization | TCCAACCCAGG | GAAAGTGACAG | TCTCTAATCTG | GCTCTTTGTCT |
| probe 2 | TTTGT | CTGTC | TTAGTGCA | TCTC |
| | (SEQ ID NO: 26) | (SEQ ID NO: 27) | (SEQ ID NO: 28) | (SEQ ID NO: 29) |

One embodiment of the genotyping method involves isolating from the individual a biological sample comprising nucleic acids, isolating from the sample the two copies of the CCL2 gene, or a fragment thereof, and determining the identity of the nucleotide pair at one or more polymorphic sites within the CCL2 gene. The polymorphic sites can be, for example, the nucleotides or alleles at positions 2236, 2485, 2936 or 5837 as numbered in SEQ ID NO:1, or polymorphic sites which are in linkage disequilibrium with any of the above. As used herein, the two “copies” of a gene in an individual may be the same allele or may be different alleles. In one embodiment of the genotyping method, the identity of the nucleotide pair at one or more of the polymorphic sites described above is determined. In another embodiment, the genotyping method comprises determining the identity of the nucleotide pair at the nucleotide or allele at position 2485 as numbered in SEQ ID NO:1.

The nucleic acid sample may be comprised of genomic DNA, mRNA, or cDNA and, in the latter two cases, the biological sample must be obtained from a tissue in which the CCL2 gene is expressed. Furthermore it will be understood by the skilled artisan that mRNA or cDNA preparations would not be used to detect polymorphisms located in introns or in 5′ and 3′ untranslated regions. If a CCL2 gene fragment is isolated, it must contain the polymorphic site(s) to be genotyped.

The haplotyping method comprises a method for assigning a CCL2 haplotype to an individual by isolating from the individual a nucleic acid sample containing only one of the two copies of the CCL2 gene, or a fragment thereof, that is present in the individual and determining in that copy the identity of the nucleotide at one or more polymorphic sites. The polymorphic sites can be, for example, the nucleotide or allele at positions 2236, 2485, 2936 or 5837 as numbered in SEQ ID NO:1, or polymorphic sites which are in linkage disequilibrium with any of the above.

One embodiment of the haplotyping method comprises determining whether an individual has one or more of the CCL2 haplotypes shown in Table II below. This can be accomplished by identifying, for one or both copies of the individual's CCL2 gene, the sequence of nucleotides present at each of the nucleotides or alleles at positions 2236, 2485, 2936, or 5837 as numbered in SEQ ID NO:1, or the sequence of nucleotides which are in linkage disequilibrium with any of the above.

The present invention also contemplates that typically only a subset of the nucleotides or alleles at positions 2236, 2485, 2936, or 5837 as numbered in SEQ ID NO:1, or the nucleotides which are in linkage disequilibrium with any of the above, will need to be directly examined to assign to an individual one or more of the haplotypes shown in Table II. This is because at least one polymorphic site in a gene is frequently in strong linkage disequilibrium with one or more other polymorphic sites in that gene (Drysdale, C M et al. 2000 Prot. Natl Acad. Sci USA 97:10483-10488; Rieder M J et al. 1999 Nature Genetics 22:59-62).

TABLE II
(McDermott et al. 2005 Circulation 112: 1113–1120)

| | | | | Position |
|---|---|---|---|---|
| Haplo- | Position 2236 | Position 2485 | Position 2936 | 5837 |
| type | (rs2857654) | (rs1024611) | (rs1024610) | (rs2857657) |
| H1 | A | G | A | C |
| H2 | C | A | T | G |
| H3 | C | A | A | C |

In a preferred embodiment, a CCL2 haplotype pair is determined for an individual by identifying the sequence of nucleotides at one or more polymorphic sites selected from the group consisting of the nucleotide at position 2236 as numbered in SEQ ID NO:1, the nucleotide at position 2485 as numbered in SEQ ID NO:1, the nucleotide at position 2936 as numbered in SEQ ID NO:1, or the nucleotide at position 5837 as numbered in SEQ ID NO:1, or any nucleotides or alleles which are in linkage disequilibrium with any of the above, in each copy of the CCL2 gene that is present in the individual. In a preferred embodiment, the haplotyping method comprises identifying the sequence of nucleotides at each of the nucleotides at positions 2236, 2485, 2936, or 5837 as numbered in SEQ ID NO:1, or the nucleotides which are in linkage disequilibrium with any of the above, in each copy of the CCL2 gene. When haplotyping both copies of the gene, the identifying step is preferably performed with each copy of the gene being placed in separate containers. However, it is also envisioned that if the two copies are labeled with different tags, or are otherwise separately distinguishable or identifiable, it could be possible in some cases to perform the method in the same container. For example, if first and second copies of the gene are labeled with different first and second fluorescent dyes, respectively, and an allele-specific oligonucleotide labeled with yet a third different fluorescent dye is used to assay the polymorphic site(s), then detecting a combination of the first and third dyes would identify the polymorphism in the first gene copy while detecting a combination of the second and third dyes would identify the polymorphism in the second gene copy.

In both the genotyping and haplotyping methods, the identity of a nucleotide (or nucleotide pair) at a polymorphic site(s) may be determined by amplifying a target region(s) containing the polymorphic site(s) directly from one or both copies of the CCL2 gene, or a fragment thereof, and the sequence of the amplified region(s) determined by conventional methods. It will be readily appreciated by the skilled artisan that only one nucleotide will be detected at a polymorphic site in individuals who are homozygous at that site, while two different nucleotides will be detected if the individual is heterozygous for that site. The polymorphism may be identified directly, known as positive-type identification, or by inference, referred to as negative-type identification. For example, where a SNP is known to be guanine and cytosine in a reference population, a site may be positively determined to be either guanine or cytosine for an individual homozygous at that site, or both guanine and cytosine, if the individual is heterozygous at that site. Alternatively, the site may be negatively determined to be not guanine (and thus cytosine/cytosine) or not cytosine (and thus guanine/guanine).

The target region(s) may be amplified using any oligonucleotide-directed amplification method, including but not limited to polymerase chain reaction (PCR) (U.S. Pat. No. 4,965,188), ligase chain reaction (LCR) (Barany et al., Proc. Natl. Acad. Sci. USA 88:189-193, 1991; WO90/01069), and oligonucleotide ligation assay (OLA) (Landegren et al., Science 241:1077-1080, 1988).

Other known nucleic acid amplification procedures may be used to amplify the target region including transcription-based amplification systems (U.S. Pat. No. 5,130,238; EP 329,822; U.S. Pat. No. 5,169,766, WO89/06700) and isothermal methods (Walker et al., Proc. Natl. Acad. Sci. USA 89:392-396, 1992).

A polymorphism in the target region may also be assayed before or after amplification using one of several hybridization-based methods known in the art. Typically, allele-specific oligonucleotides are utilized in performing such methods. The allele-specific oligonucleotides may be used as differently labeled probe pairs, with one member of the pair showing a perfect match to one variant of a target sequence and the other member showing a perfect match to a different variant. In some embodiments, more than one polymorphic site may be detected at once using a set of allele-specific oligonucleotides or oligonucleotide pairs. Preferably, the members of the set have melting temperatures within five degrees centigrade, and more preferably within two degrees centigrade, of each other when hybridizing to each of the polymorphic sites being detected.

Hybridization of an allele-specific oligonucleotide to a target polynucleotide may be performed with both entities in solution, or such hybridization may be performed when either the oligonucleotide or the target polynucleotide is covalently or non-covalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibers, chips, dishes, and beads. The solid support may be treated, coated or derivatized to facilitate the immobilization of the allele-specific oligonucleotide or target nucleic acid.

The genotype or haplotype for the CCL2 gene of an individual may also be determined by hybridization of a nucleic acid sample containing one or both copies of the gene, or fragment(s) thereof, to nucleic acid arrays. The arrays may contain a plurality of allele-specific oligonucleotides representing each of the polymorphic sites to be included in the genotype or haplotype.

The identity of polymorphisms may also be determined using a mismatch detection technique, including but not limited to the RNase protection method using riboprobes (Winter et al., Proc. Natl. Acad Sci. USA 82:7575, 1985; Meyers et al., Science 230:1242, 1985) and proteins which recognize nucleotide mismatches, such as the E. coli mutS protein (Modrich, P. Ann. Rev. Genet. 25:229-253, 1991). Alternatively, variant alleles can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et al., Nuci. Acids Res. 18:2699-2706, 1990; Sheffield et al., Proc. Nati. Acad. Sci. USA 86:232-236, 1989).

A polymerase-mediated primer extension method may also be used to identify the polymorphism(s). Several such methods have been described in the patent and scientific literature and include the “Genetic Bit Analysis” method (WO92/15712) and the ligase/polymerase mediated genetic bit analysis (U.S. Pat. No. 5,679,524). Related methods are disclosed in WO91/02087, WO90/09455, WO95/17676, U.S. Pat. Nos. 5,302,509, and 5,945,283. Extended primers containing a polymorphism may be detected by mass spectrometry as described in U.S. Pat. No. 5,605,798. Another primer extension method is allele-specific PCR (Ruano et al., Nucl. Acids Res. 17:8392, 1989; Ruano et al., Nucl. Acids Res. 19, 6877-6882, 1991; WO 93/22456; Turki et al., J Clin. Invest. 95:1635-1641, 1995). In addition, multiple polymorphic sites may be investigated by simultaneously amplifying multiple regions of the nucleic acid using sets of allele-specific primers as described in Wallace et al. (WO89/10414).

As mentioned throughout, the identity of the allele(s) present at any of the polymorphic sites described herein may be indirectly determined by genotyping another polymorphic site that is in linkage disequilibrium with the polymorphic site that is of interest. Polymorphic sites in linkage disequilibrium with the presently disclosed polymorphic sites may be located in regions of the gene or in other genomic regions not examined herein. Genotyping of a polymorphic site in linkage disequilibrium with the novel polymorphic sites described herein may be performed by, but is not limited to, any of the above-mentioned methods for detecting the identity of the allele at a polymorphic site.

The methods for evaluating a patient having a CCR-2 mediated disorder, e.g., a CCR-2 mediated disorder described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, can include an additional step of recording the value of a parameter related to the patient's CCL2 expression level or recording the patient's genotype.

“Recording” as used herein, refers to the act or process of making a record capable of being accessed or referenced at a later date. In one embodiment, the record is made in writing. In one embodiment, the record is made on paper (e.g., written in a patient's medical record or written on a batch record), or the record is made in an electronic medium (e.g., the record is entered into a computer, for example, the record is entered into an electronic version of the patient's medical record or the record is entered into a database). In another embodiment, the record is made vocally by recording one's voice. In one embodiment, the voice recording is made on, for example, a tape or compact disk. In one embodiment, the recorded information contains reference standard value(s).

The methods for evaluating a patient having a CCR-2 mediated disorder for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, can include a further step of determining, recommending or selecting an appropriate treatment regimen.

As used herein, “determining an appropriate treatment regimen” refers to the act or process of reviewing a patient's value for a parameter related to the patient's CCL2 expression level and comparing this value to a reference standard and/or reviewing the patient's genotype; and, optionally, reviewing the patient's medical history (e.g., for allergies or intolerances to certain types of drugs, or for drug incompatibilities) and assessing the likelihood that the patient will be responsive to a given treatment regimen.

As used herein, “recommending an appropriate treatment regimen” refers to the act or process of suggesting, for example, to the patient, to a family member or caregiver of the patient, or to medical personnel (e.g., the patient's primary care physician), a treatment regimen which is perceived as being favorable for the patient. As used herein, a recommendation can be a written or a verbal recommendation.

As used herein, “selecting an appropriate treatment regimen” refers to the act or process of picking or choosing a treatment regimen from other treatment regimen options for a patient. In one embodiment, the selection is made upon review of i) a patient's value for a parameter related to the patient's CCL2 expression level and comparing this value to a reference standard; ii) the patient's genotype; and/or iii) the patient's medical history (e.g., for allergies or intolerances to certain types of drugs, for drug incompatibilities and for treatment history) and assessing the likelihood that the patient will be responsive to a given treatment regimen. In another embodiment, the selection is made based upon a recommendation.

In one embodiment, the act or process of determining, recommending or selecting an appropriate treatment regimen is based upon the review of a patient's value for a parameter related to the patient's CCL2 expression level and comparing this value to a reference standard in order to make an informed decision. In one embodiment, the value may be determined according to any of the detection methods described herein. In one embodiment, the test results may indicate that an individual's CCL2 expression level is not significantly greater than the value of the reference standard for homozygous A individuals. In such cases, no treatment regimen or a treatment regimen that does not include a CCR-2 antagonist and/or a CCL2 antagonist may be recommended or selected. Alternatively, a treatment regimen comprising i) a CCR-2 antagonist or CCL2 antagonist which is administered at an increased dose and/or an increased administration schedule, e.g., at a dose statistically greater than recommended for patients having the disorder and/or at a dosing frequency greater than recommended for patients having the disorder, ii) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, iii) a treatment regimen comprising a CCR-2 antagonist, e.g., a CCR-2 antagonist described herein, in combination with an agent other than a CCR-2 antagonist; or iv) a treatment regimen comprising a CCL2 antagonist, e.g., a CCL2 antagonist described herein, in combination with an agent other than a CCL2 antagonist, may be recommended or selected.

In another embodiment, the test results may indicate that an individual's CCL2 expression level is over-expressed in comparison to the reference standard for homozygous A individuals. In another embodiment, the test results may indicate that an individual's CCL2 expression level is relatively the same as the reference standard for homozygous G or heterozygous individuals. In yet another embodiment, the test results may indicate that an individual's CCL2 expression level is over-expressed in comparison to the reference standard for homozygous G or heterozygous individuals. In such cases, the treatment regimen recommended or selected may be: i) an agent or compound which inhibits the interaction between CCR-2 and CCL2, e.g., an agent or compound which inhibits the interaction between CCR-2 and CCL2 as described herein; ii) a CCR-2 antagonist, e.g., a CCR-2 antagonist described herein, iii) a CCL2 antagonist, e.g., a CCL2 antagonist, as described herein, iv) a treatment regimen comprising a CCR-2 antagonist or CCL2 antagonist which is administered at an increased dose and/or an increased administration schedule, e.g., at a dose statistically greater than recommended for patients having the disorder and/or at a dosing frequency greater than recommended for patients having the disorder, v) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, vi) a treatment regimen comprising a CCR-2 antagonist, e.g., a CCR-2 antagonist described herein, in combination with an agent other than a CCR-2 antagonist; vii) a treatment regimen comprising a CCL2 antagonist, e.g., a CCL2 antagonist described herein, in combination with an agent other than a CCL2 antagonist; or viii) a combination of any of the above treatment regimens.

In one embodiment, the patient's genotype in the patient's CCL2 genomic sequence is also reviewed in connection with the review of the patient's value for a parameter related to the patient's CCL2 expression level. In another embodiment, the patient's medical history is also reviewed in connection with the review of the patient's value for a parameter related to the patient's CCL2 expression level.

In one embodiment, the act or process of determining, recommending or selecting an appropriate treatment regimen is based upon the review of a patient's genotype at a pre-selected nucleotide, allele or combination of alleles within the patient's CCL2 genomic sequence, alone or in combination with another predictive indicator or marker. In one embodiment, the patient's genotype is determined according to any of the diagnostic methods (e.g., genotyping or haplotyping methods) described herein. In one embodiment, the pre-selected nucleotide, allele or combination of alleles is/are within a non-coding region (e.g., an intron or a regulatory region) of the CCL2 genomic sequence. In one embodiment, the pre-selected nucleotide, allele or combination of alleles is a nucleotide at position 2236, 2485, 2936 or 5837 as numbered in SEQ ID NO:1, or one or more nucleotides, allele or combination of alleles in linkage disequilibrium with any of the above. In one embodiment, the pre-selected nucleotide or allele in the patient's CCL2 genomic sequence at position 2485 is a G. In another embodiment, the pre-selected nucleotide or allele in the patient's CCL2 genomic sequence at position 2236 is an A. In another embodiment, the pre-selected nucleotide or allele in the patient's CCL2 genomic sequence at position 2936 is an A. In yet another embodiment, the pre-selected nucleotide or allele in the patient's CCL2 genomic sequence at position 5837 is a C. In another embodiment, the patient exhibits haplotype H1, as described in Table II above, in the patient's CCL2 genomic sequence. In one embodiment, when the G allele is present at the nucleotide at position 2485 as numbered in SEQ ID NO:1, the treatment regimen selected or recommended may be i) an agent or compound which inhibits the interaction between CCR-2 and CCL2, e.g., an agent or compound which inhibits the interaction between CCR-2 and CCL2 as described herein; ii) a CCR-2 antagonist, e.g., a CCR-2 antagonist described herein; iii) a CCL2 antagonist, e.g., a CCL2 antagonist described herein; iv) a treatment regimen comprising a CCR-2 antagonist, e.g., a CCR-2 antagonist described herein, in combination with an agent other than a CCR-2 antagonist; v) a treatment regimen comprising a CCL2 antagonist, e.g., a CCL2 antagonist described herein, in combination with an agent other than a CCL2 antagonist; vi) a treatment regimen comprising a CCR-2 antagonist or CCL2 antagonist which is administered at an increased dose and/or an increased administration schedule, e.g., at a dose statistically greater than recommended for patients having the disorder and/or at a dosing frequency greater than recommended for patients having the disorder, vii) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder; or viii) a combination of any of the above treatment regimens. Alternatively, when the A allele is present at the nucleotides at positions 2236 or 2936, as numbered in SEQ D NO:1; when the C allele is present at the nucleotide at position 5837, as numbered in SEQ ID NO:1; or a nucleotide or allele in linkage disequilibrium with any of the above; or when the patient exhibits haplotype H1, as defined in Table II above, in the patient's CCL2 genomic sequence, then the same treatment regimens as described above for when the G allele is present at position 2485, can be selected or recommended.

In yet another embodiment, the pre-selected nucleotide or allele in the patient's CCL2 genomic sequence at position 2485, as numbered in SEQ ID NO:1, is homozygous A. In one embodiment, when the patient has a homozygous A allele at the nucleotide at position 2485, the treatment regimen selected or recommended is either i) no treatment regimen; ii) a treatment regimen other than a CCR-2 antagonist; iii) a treatment regimen other than a CCL2 antagonist; iv) a treatment regimen comprising a CCR-2 antagonist, e.g., a CCR-2 antagonist described herein, in combination with an agent other than a CCR-2 antagonist; v) a treatment regimen comprising a CCL2 antagonist, e.g., a CCL2 antagonist described herein, in combination with an agent other than a CCL2 antagonist; vi) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vii) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In one embodiment, when the CCR-2 mediated disorder is a cardiovascular disorder, for example, atherosclerosis, the treatment regimen other than a CCR-2 antagonist or other than a CCL2 antagonist is a treatment regimen using HMG-CoA reductase inhibitors, Statins, Fibrates, ACE inhibitors, anti-hypertensives, anti-thrombotics, β blockers, anti-diabetes drugs or anti-obesity drugs. In one embodiment, the HMG-CoA reductase inhibitor or Statin is selected from the group consisting of Atorvastatin, Cerivastatin, Fluvastatin, Lovastatin, Mevastatin, Pitavastatin, Pravastatin, Rosuvastatin, and Simvastatin.

In one embodiment, the patient's medical history is also reviewed in connection with the review of the patient's genotype.

In one embodiment, the determination, recommendation or selection of an appropriate treatment regimen for a patient is based upon the review of the patient's value for a parameter related to the patient's CCL2 expression level, the patient's genotype and the patient's medical history.

In one embodiment, the patient is then treated, as described further, according to the recommended or selected treatment regimen.

The methods for evaluating a patient having a CCR-2 mediated disorder, e.g., a CCR-2 mediated disorder as described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, typically include an alternative step of determining whether to begin, continue, discontinue, change or alter the treatment regimen based upon the patient's CCL2 expression level or genotype.

When a patient has been diagnosed with a CCR-2 mediated disorder and has not started any treatment regimen, the above methods are suited to determine whether the patient is likely to respond to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, by determining the patient's value for a parameter related to the patient's CCL2 expression level, and/or by determining the patient's genotype at a pre-selected nucleotide, allele or combination of alleles in the patient's CCL2 genomic sequence as described in detail above.

In one embodiment, when the test results indicate that an individual's CCL2 expression level is not significantly greater than the value of the reference standard for homozygous A individuals, it may be determined to not give the patient any treatment regimen or it may be determined to begin a treatment regimen other than a CCR-2 antagonist treatment regimen or other than a CCL2 antagonist treatment regimen. Alternatively, it may be determined to begin a treatment regimen comprising i) a CCR-2 antagonist or CCL2 antagonist which is administered at an increased dose and/or an increased administration schedule, e.g., at a dose statistically greater than recommended for patients having the disorder and/or at a dosing frequency greater than recommended for patients having the disorder, ii) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, iii) a treatment regimen comprising a CCR-2 antagonist, e.g., a CCR-2 antagonist described herein, in combination with an agent other than a CCR-2 antagonist; or iv) a treatment regimen comprising a CCL2 antagonist, e.g., a CCL2 antagonist described herein, in combination with an agent other than a CCL2 antagonist.

In another embodiment, when the test results indicate that the patient's CCL2 expression level is over-expressed in comparison to the reference standard for homozygous A individuals; when the test results indicate that the patient's CCL2 expression level is relatively the same as the reference standard for homozygous G or heterozygous individuals; or when the test results indicate that the patient's CCL2 expression level is over-expressed in comparison to the reference standard for homozygous G or heterozygous individuals, it may be determined to begin a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof as described herein.

In one embodiment, the patient's genotype in the patient's CCL2 genomic sequence is also reviewed in connection with the review of the patient's value for a parameter related to the patient's CCL2 expression level. In another embodiment, the patient's medical history is also reviewed in connection with the review of the patient's value for a parameter related to the patient's CCL2 expression level.

In one embodiment, when the patient has a G allele at the nucleotide or allele at position 2485, as numbered in SEQ ID NO:1, or a nucleotide or allele in linkage disequilibrium with the nucleotide or allele at position 2485, it may be determined to begin a treatment regimen selected from the group consisting of i) an agent or compound which inhibits the interaction between CCR-2 and CCL2, e.g., an agent or compound which inhibits the interaction between CCR-2 and CCL2 as described herein; ii) a CCR-2 antagonist, e.g., a CCR-2 antagonist described herein; iii) a CCL2 antagonist, e.g., a CCL2 antagonist described herein; iv) a treatment regimen comprising a CCR-2 antagonist or CCL2 antagonist which is administered at an increased dose and/or an increased administration schedule, e.g., at a dose statistically greater than recommended for patients having the disorder and/or at a dosing frequency greater than recommended for patients having the disorder; v) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder; vi) a treatment regimen comprising a CCR-2 antagonist, e.g., a CCR-2 antagonist described herein, in combination with an agent other than a CCR-2 antagonist; or vii) a treatment regimen comprising a CCL2 antagonist, e.g., a CCL2 antagonist described herein, in combination with an agent other than a CCL2 antagonist.

Alternatively, when the A allele is present at the nucleotides at positions 2236 or 2936, as numbered in SEQ D NO:1; when the C allele is present at the nucleotide at position 5837, as numbered in SEQ ID NO:1; or nucleotides or alleles in linkage disequilibrium with any of the above; or when the patient exhibits haplotype H1, as defined in Table II above, in the patient's CCL2 genomic sequence, then it may be determined to begin the same treatment regimens as described above for when the G allele is present at position 2485.

In yet another embodiment, when the patient has a homozygous A allele at the nucleotide at position 2485, or a nucleotide or allele in linkage disequilibrium with the nucleotide or allele at position 2485, it may be determined to i) not begin any treatment regimen; ii) begin a treatment regimen other than a CCR-2 antagonist; iii) begin a treatment regimen other than a CCL2 antagonist; iv) begin a treatment regimen comprising a CCR-2 antagonist, e.g., a CCR-2 antagonist described herein, in combination with an agent other than a CCR-2 antagonist; v) begin a treatment regimen comprising a CCL2 antagonist, e.g., a CCL2 antagonist described herein, in combination with an agent other than a CCL2 antagonist; vi) begin a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vii) begin a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In one embodiment, when the CCR-2 mediated disorder is a cardiovascular disorder, for example, atherosclerosis, the treatment regimen other than a CCR-2 antagonist or other than a CCL2 antagonist is a treatment regimen using HMG-CoA reductase inhibitors, Statins, Fibrates, ACE inhibitors, anti-hypertensives, anti-thrombotics, β blockers, anti-diabetes drugs or anti-obesity drugs. In one embodiment, the HMG-CoA reductase inhibitor or Statin is selected from the group consisting of Atorvastatin, Cerivastatin, Fluvastatin, Lovastatin, Mevastatin, Pitavastatin, Pravastatin, Rosuvastatin, and Simvastatin.

In one embodiment, the patient's medical history is also reviewed in connection with the review of the patient's genotype.

In one embodiment, the determination of whether to begin a treatment regimen for a patient is based upon the review of the patient's value for a parameter related to the patient's CCL2 expression level, the patient's genotype and the patient's medical history.

In one embodiment, the patient is then treated, as described further, according to the determination regarding the treatment regimen.

In another embodiment, when a patient having a CCR-2 mediated disorder has already begun a CCR-2 antagonist treatment regimen or a CCL2 antagonist treatment regimen, the above methods are suited to determine whether the patient's current treatment regimen is appropriate or whether it should be continued, discontinued, changed or altered.

In one embodiment, when the test results indicate that an individual's CCL2 expression level is not significantly greater than the value of the reference standard for homozygous A individuals, it may be determined to discontinue, change or alter the treatment regimen. In one embodiment, a change or alteration of the treatment regimen may comprise i) changing the treatment regimen to a treatment regimen other than a CCR-2 antagonist; ii) changing the treatment regimen to a treatment regimen other than a CCL2 antagonist; iii) changing the treatment regimen to a treatment regimen comprising a CCR-2 antagonist, e.g., a CCR-2 antagonist described herein, in combination with an agent other than a CCR-2 antagonist; iv) changing the treatment regimen to a treatment regimen comprising a CCL2 antagonist, e.g., a CCL2 antagonist described herein, in combination with an agent other than a CCL2 antagonist; v) changing the treatment regimen to a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vi) changing the treatment regimen to a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In another embodiment, when the test results indicate that the patient's CCL2 expression level is over-expressed in comparison to the reference standard for homozygous A individuals; when the test results indicate that the patient's CCL2 expression level is relatively the same as the reference standard for homozygous G or heterozygous individuals; or when the test results indicate that the patient's CCL2 expression level is over-expressed in comparison to the reference standard for homozygous G or heterozygous individuals, it may be determined to continue the CCR-2 antagonist or the CCL2 antagonist. However, it may be determined that the treatment regimen be continued by altering the treatment regimen to maximize responsiveness to the treatment regimen by i) altering the treatment regimen to a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or ii) altering the treatment regimen to a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder; iii) altering the treatment regimen to a treatment regimen comprising a CCR-2 antagonist, e.g., a CCR-2 antagonist described herein, in combination with an agent other than a CCR-2 antagonist; or iv) altering the treatment regimen to a treatment regimen comprising a CCL2 antagonist, e.g., a CCL2 antagonist described herein, in combination with an agent other than a CCL2 antagonist.

In one embodiment, the patient's genotype in the patient's CCL2 genomic sequence is also reviewed in connection with the review of the patient's value for a parameter related to the patient's CCL2 expression level. In another embodiment, the patient's medical history is also reviewed in connection with the review of the patient's value for a parameter related to the patient's CCL2 expression level.

In one embodiment, when the patient has a G allele at the nucleotide at position 2485, as numbered in SEQ ID NO:1, or a nucleotide or allele in linkage disequilibrium with the nucleotide at position 2485, it may be determined to continue the CCR-2 antagonist or the CCL2 antagonist. However, it may be determined that the treatment regimen be continued by altering the treatment regimen to maximize responsiveness to the treatment regimen by i) altering the treatment regimen to a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder; ii) altering the treatment regimen to a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder; iii) altering the treatment regimen to a treatment regimen comprising a CCR-2 antagonist, e.g., a CCR-2 antagonist described herein, in combination with an agent other than a CCR-2 antagonist; or iv) altering the treatment regimen to a treatment regimen comprising a CCL2 antagonist, e.g., a CCL2 antagonist described herein, in combination with an agent other than a CCL2 antagonist.

Alternatively, when the A allele is present at the nucleotides at positions 2236 or 2936, as numbered in SEQ D NO:1; when the C allele is present at the nucleotide at position 5837, as numbered in SEQ ID NO:1; or nucleotides or alleles in linkage disequilibrium with any of the above; or when the patient exhibits haplotype H1, as defined in Table II above, in the patient's CCL2 genomic sequence, then it may be determined to continue with the same treatment regimen as described above for when the G allele is present at position 2485, or it may be determined to alter the treatment regimen as described above to maximize response rate.

In yet another embodiment, when the patient has a homozygous A allele at the nucleotide at position 2485, as numbered in SEQ ID NO:1, or a nucleotide in linkage disequilibrium with the nucleotide at position 2485, it may be determined to discontinue, change or alter the treatment regimen. In one embodiment, a change or alteration of the treatment regimen may comprise i) changing the treatment regimen to a treatment regimen other than a CCR-2 antagonist; ii) changing the treatment regimen to a treatment regimen other than a CCL2 antagonist; iii) changing the treatment regimen to a treatment regimen comprising a CCR-2 antagonist, e.g., a CCR-2 antagonist described herein, in combination with an agent other than a CCR-2 antagonist; iv) changing the treatment regimen to a treatment regimen comprising a CCL2 antagonist, e.g., a CCL2 antagonist described herein, in combination with an agent other than a CCL2 antagonist; v) changing the treatment regimen to a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vi) changing the treatment regimen to a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In one embodiment, when the CCR-2 mediated disorder is a cardiovascular disorder, for example, atherosclerosis, the treatment regimen other than a CCR-2 antagonist or other than a CCL2 antagonist is a treatment regimen using HMG-CoA reductase inhibitors, Statins, Fibrates, ACE inhibitors, anti-hypertensives, anti-thrombotics, β blockers, anti-diabetes drugs or anti-obesity drugs. In one embodiment, the HMG-CoA reductase inhibitor or Statin is selected from the group consisting of Atorvastatin, Cerivastatin, Fluvastatin, Lovastatin, Mevastatin, Pitavastatin, Pravastatin, Rosuvastatin, and Simvastatin.

In one embodiment, the patient's medical history is also reviewed in connection with the review of the patient's genotype.

In one embodiment, the determination of whether to continue, discontinue, change or alter a treatment regimen for a patient is based upon the review of the patient's value for a parameter related to the patient's CCL2 expression level, the patient's genotype and the patient's medical history.

In one embodiment, the patient is then treated, as described further, according to the determination regarding the treatment regimen.

### Selecting a Treatment Regimen for a Patient Having a CCR-2 Mediated Disorder.

The methods for selecting a treatment regimen for a patient having a CCR-2 mediated disorder, e.g., a CCR-2 mediated disorder as described herein, typically include a step of determining or confirming a value for a parameter related to a patient's CCL2 expression level and/or determining or confirming a patient's genotype at a pre-selected nucleotide within the patient's CCL2 genomic sequence, followed by a step of recording the value or genotype, then followed by a step of selecting an appropriate treatment regimen based upon the value or the genotype. In one embodiment, the method is an in vitro method. In another embodiment, the method is an in vivo method

As described elsewhere herein, “selecting an appropriate treatment regimen” refers to the act or process of picking or choosing a treatment regimen for a patient.

In one embodiment, the act or process of selecting an appropriate treatment regimen is based upon the review of a patient's value for a parameter related to the patient's CCL2 expression level and comparing this value to a reference standard in order to make an informed decision for the selection. In one embodiment, the value may be determined according to any of the detection methods described herein. In one embodiment, the test results can indicate that an individual's CCL2 expression level is not significantly greater than the value of the reference standard for homozygous A individuals. In such cases, the selected treatment regimen may be selected from the group consisting of: i) no treatment regimen; ii) a treatment regimen other than a CCR-2 antagonist; iii) a treatment regimen other than a CCL2 antagonist; iv) a treatment regimen comprising a CCR-2 antagonist, e.g., a CCR-2 antagonist described herein, in combination with an agent other than a CCR-2 antagonist; v) a treatment regimen comprising a CCL2 antagonist, e.g., a CCL2 antagonist described herein, in combination with an agent other than a CCL2 antagonist; vi) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vii) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In another embodiment, the test results may indicate that an individual's CCL2 expression level is over-expressed in comparison to the reference standard for homozygous A individuals. In another embodiment, the test results may indicate that an individual's CCL2 expression level is relatively the same as the reference standard for homozygous G or heterozygous individuals. In yet another embodiment, the test results may indicate that an individual's CCL2 expression level is over-expressed in comparison to the reference standard for homozygous G or heterozygous individuals. In such cases, the treatment regimen selected may be selected from the group consisting of: i) an agent or compound which inhibits the interaction between CCR-2 and CCL2, e.g., an agent or compound which inhibits the interaction between CCR-2 and CCL2 as described herein; ii) a CCR-2 antagonist, e.g., a CCR-2 antagonist described herein, iii) a CCL2 antagonist, e.g., a CCL2 antagonist, as described herein; iv) a treatment regimen comprising a CCR-2 antagonist, e.g., a CCR-2 antagonist described herein, in combination with an agent other than a CCR-2 antagonist; v) a treatment regimen comprising a CCL2 antagonist, e.g., a CCL2 antagonist described herein, in combination with an agent other than a CCL2 antagonist; vi) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, and vii) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In one embodiment, the patient's genotype in the patient's CCL2 genomic sequence is also reviewed in connection with the review of the patient's value for a parameter related to the patient's CCL2 expression level. In another embodiment, the patient's medical history is also reviewed in connection with the review of the patient's value for a parameter related to the patient's CCL2 expression level.

In one embodiment, the act or process of selecting an appropriate treatment regimen is based upon the review of a patient's genotype at a pre-selected nucleotide, allele or combination of alleles, or at a nucleotide, allele or combination of alleles in linkage disequilibrium with the pre-selected nucleotide, allele or combination of alleles, within the patient's CCL2 genomic sequence. In one embodiment, the patient's genotype is determined according to any of the diagnostic methods (e.g., genotyping or haplotyping methods) described herein. In one embodiment, the pre-selected nucleotide, allele or combination of alleles is/are within a non-coding region (e.g., an intron or a regulatory region) of the CCL2 genomic sequence. In one embodiment, the pre-selected nucleotide, allele or combination of alleles is a nucleotide at position 2236, 2485, 2936 or 5837 as numbered in SEQ ID NO:1, or one or more nucleotides, allele or combination of alleles in linkage disequilibrium with any of the above-listed nucleotides. In one embodiment, the pre-selected nucleotide or allele in the patient's CCL2 genomic sequence at position 2485 is a G. In another embodiment, the pre-selected nucleotide or allele in the patient's CCL2 genomic sequence at position 2236 is an A. In another embodiment, the pre-selected nucleotide or allele in the patient's CCL2 genomic sequence at position 2936 is an A. In yet another embodiment, the pre-selected nucleotide or allele in the patient's CCL2 genomic sequence at position 5837 is a C. In another embodiment, the patient exhibits haplotype H1, as described in Table II above, in the patient's CCL2 genomic sequence. In one embodiment, when the G allele is present at the nucleotide at position 2485 as numbered in SEQ ID NO:1, or a nucleotide or allele in linkage disequilibrium with the nucleotide at position 2485, the treatment regimen selected may be i) an agent or compound which inhibits the interaction between CCR-2 and CCL2, e.g., an agent or compound which inhibits the interaction between CCR-2 and CCL2 as described herein; ii) a CCR-2 antagonist, e.g., a CCR-2 antagonist described herein, iii) a CCL2 antagonist, e.g., a CCL2 antagonist, as described herein; iv) a treatment regimen comprising a CCR-2 antagonist, e.g., a CCR-2 antagonist described herein, in combination with an agent other than a CCR-2 antagonist; v) a treatment regimen comprising a CCL2 antagonist, e.g., a CCL2 antagonist described herein, in combination with an agent other than a CCL2 antagonist; vi) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vii) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

Alternatively, when the A allele is present at the nucleotides at positions 2236 or 2936, as numbered in SEQ D NO:1, or a nucleotide or allele in linkage disequilibrium with the nucleotides at positions 2236 or 2936; when the C allele is present at the nucleotide at position 5837, as numbered in SEQ ID NO:1, or a nucleotide or allele in linkage disequilibrium with the nucleotide at position 5837, as numbered in SEQ ID NO:1; or when the patient exhibits haplotype H1, as defined in Table II above, in the patient's CCL2 genomic sequence, then the same treatment regimens as described above for when the G allele is present at position 2485, may be selected.

In yet another embodiment, the pre-selected nucleotide in the patient's CCL2 genomic sequence at position 2485, as numbered in SEQ ID NO:1, is homozygous A. In one embodiment, when the patient has a homozygous A allele at the nucleotide at position 2485, or a nucleotide or allele in linkage disequilibrium with the nucleotide or allele at position 2485, the treatment regimen selected is either i) no treatment regimen; ii) a treatment regimen other than a CCR-2 antagonist; iii) a treatment regimen other than a CCL2 antagonist; iv) a treatment regimen comprising a CCR-2 antagonist, e.g., a CCR-2 antagonist described herein, in combination with an agent other than a CCR-2 antagonist; v) a treatment regimen comprising a CCL2 antagonist, e.g., a CCL2 antagonist described herein, in combination with an agent other than a CCL2 antagonist; vi) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vii) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In one embodiment, when the CCR-2 mediated disorder is a cardiovascular disorder, for example, atherosclerosis, the treatment regimen other than a CCR-2 antagonist or other than a CCL2 antagonist is a treatment regimen using HMG-CoA reductase inhibitors, Statins, Fibrates, ACE inhibitors, anti-hypertensives, anti-thrombotics, β blockers, anti-diabetes drugs or anti-obesity drugs. In one embodiment, the HMG-CoA reductase inhibitor or Statin is selected from the group consisting of Atorvastatin, Cerivastatin, Fluvastatin, Lovastatin, Mevastatin, Pitavastatin, Pravastatin, Rosuvastatin, and Simvastatin.

In one embodiment, the patient's medical history is also reviewed in connection with the review of the patient's genotype.

In one embodiment, the selection of an appropriate treatment regimen for a patient is based upon the review of the patient's value for a parameter related to the patient's CCL2 expression level, the patient's genotype and the patient's medical history.

In one embodiment, the patient is then treated, as described further, according to the selected treatment regimen.

### Selecting a Patient Population for Treatment With a Treatment Regimen Comprising an Agent or Compound Which Inhibits the Interaction Between CCR-2 and CCL2; a CCR-2 Antagonist; a CCL2 Antagonist; or a Combination Thereof.

The methods for selecting a patient population for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, can include a step of providing a population of patients having a CCR-2 mediated disorder, e.g., a CCR-2 mediated disorder as described herein, followed by a step of determining a patient's value for a parameter related to the patient's CCL2 expression level, and/or determining a patient's genotype at a pre-selected nucleotide within the patient's CCL2 genomic sequence, e.g., followed by a step of selecting one or more patients for treatment based upon the patient's CCL2 expression level in comparison to a reference standard or based upon the patient's genotype. In one embodiment, the method is an in vitro method. In another embodiment, the method is an in vivo method.

As used herein, a “population of patients having a CCR-2 mediated disorder” refers to one or more, e.g., two or more individuals having a CCR-2 mediated disorder, e.g., a CCR-2 mediated disorder as described herein. In one embodiment, the patients are organized according to population subgroups, e.g., according to the category of CCR-2 mediated disorder, e.g. according to patients having cardiovascular disorders, immune disorders, etc. In another embodiment, the patients are organized according to the patient's ethnogeographic origin.

In one embodiment of this method, only those patients believed to respond to CCR-2 antagonist treatment regimens or CCL2 antagonist treatment regimens are to be selected.

In one embodiment, the act or process of selecting a patient population for treatment with a CCR-2 antagonist treatment regimen or with a CCL2 antagonist treatment regimen is based upon the review of a patient's value for a parameter related to the patient's CCL2 expression level and comparing this value to a reference standard in order to make an informed decision for the selection. In one embodiment, the value may be determined according to any of the detection methods described herein. In one embodiment, the test results may indicate that an individual's CCL2 expression level is not significantly greater than the value of the reference standard for homozygous A individuals. In such cases, the individual is not selected for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. However, such individuals may be selected for treatment with, e.g., i) a treatment regimen comprising a CCR-2 antagonist, e.g., a CCR-2 antagonist described herein, in combination with an agent other than a CCR-2 antagonist; ii) a treatment regimen comprising a CCL2 antagonist, e.g., a CCL2 antagonist described herein, in combination with an agent other than a CCL2 antagonist; iii) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or iv) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In another embodiment, the test results may indicate that an individual's CCL2 expression level is over-expressed in comparison to the reference standard for homozygous A individuals. In another embodiment, the test results may indicate that an individual's CCL2 expression level is statistically similar to the reference standard for homozygous G or heterozygous individuals. In yet another embodiment, the test results may indicate that an individual's CCL2 expression level is over-expressed in comparison to the reference standard for homozygous G or heterozygous individuals. In such cases, the individual is selected for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, e.g., i) an agent or compound which inhibits the interaction between CCR-2 and CCL2 as described herein; ii) a CCR-2 antagonist as described herein; iii) a CCL2 antagonist as described herein; iv) a treatment regimen comprising a CCR-2 antagonist, e.g., a CCR-2 antagonist described herein, in combination with an agent other than a CCR-2 antagonist; v) a treatment regimen comprising a CCL2 antagonist, e.g., a CCL2 antagonist described herein, in combination with an agent other than a CCL2 antagonist; vi) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vii) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In one embodiment, the patient's genotype in the patient's CCL2 genomic sequence is also reviewed in connection with the review of the patient's value for a parameter related to the patient's CCL2 expression level. In another embodiment, the patient's medical history is also reviewed in connection with the review of the patient's value for a parameter related to the patient's CCL2 expression level.

In one embodiment, the act or process of selecting a patient population for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, is based upon the review of a patient's genotype at a pre-selected nucleotide, allele or combination of alleles within the patient's CCL2 genomic sequence, or at a nucleotide, allele or combination of alleles in linkage disequilibrium with the pre-selected nucleotide, allele or combination of alleles. In one embodiment, the patient's genotype is determined according to any of the diagnostic methods (e.g., genotyping or haplotyping methods) described herein. In one embodiment, the pre-selected nucleotide, allele or combination of alleles is/are within a non-coding region (e.g., an intron or a regulatory region) of the CCL2 genomic sequence. In one embodiment, the pre-selected nucleotide, allele or combination of alleles is a nucleotide at position 2236, 2485, 2936 or 5837 as numbered in SEQ ID NO:1, or one or more nucleotides, allele or combination of alleles in linkage disequilibrium with any of the above-listed nucleotides. In one embodiment, the pre-selected nucleotide or allele in the patient's CCL2 genomic sequence at position 2485 is a G. In another embodiment, the pre-selected nucleotide or allele in the patient's CCL2 genomic sequence at position 2236 is an A. In another embodiment, the pre-selected nucleotide or allele in the patient's CCL2 genomic sequence at position 2936 is an A. In yet another embodiment, the pre-selected nucleotide or allele in the patient's CCL2 genomic sequence at position 5837 is a C. In another embodiment, the patient exhibits haplotype H1, as described in Table II above, in the patient's CCL2 genomic sequence.

In one embodiment, when the G allele is present at the nucleotide at position 2485 as numbered in SEQ ID NO:1, or a nucleotide or allele in linkage disequilibrium with the nucleotide at position 2485, the patient is selected for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, e.g., i) an agent or compound which inhibits the interaction between CCR-2 and CCL2, e.g., an agent or compound which inhibits the interaction between CCR-2 and CCL2 as described herein; ii) a CCR-2 antagonist, e.g., a CCR-2 antagonist described herein, iii) a CCL2 antagonist, e.g., a CCL2 antagonist, as described herein; iv) a treatment regimen comprising a CCR-2 antagonist, e.g., a CCR-2 antagonist described herein, in combination with an agent other than a CCR-2 antagonist; v) a treatment regimen comprising a CCL2 antagonist, e.g., a CCL2 antagonist described herein, in combination with an agent other than a CCL2 antagonist; vi) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vii) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

Alternatively, when the A allele is present at the nucleotides at positions 2236 or 2936, as numbered in SEQ D NO:1, or a nucleotide or allele in linkage disequilibrium with the nucleotides at positions 2236 and 2936; when the C allele is present at the nucleotide at position 5837, as numbered in SEQ ID NO:1, or a nucleotide or allele in linkage disequilibrium with the nucleotide at position 5837; or when the patient exhibits haplotype H1, as defined in Table II above, in the patient's CCL2 genomic sequence, then the patient is also selected for treatment as described above.

In one embodiment, when the patient has a homozygous A allele at the nucleotide at position 2485, or a nucleotide or allele in linkage disequilibrium with the nucleotide or allele at position 2485, the patient is not selected for treatment with a CCR-2 antagonist treatment regimen or with a CCL2 antagonist treatment regimen. However, such individuals may be selected for treatment with, e.g., i) a treatment regimen comprising a CCR-2 antagonist, e.g., a CCR-2 antagonist described herein, in combination with an agent other than a CCR-2 antagonist; ii) a treatment regimen comprising a CCL2 antagonist, e.g., a CCL2 antagonist described herein, in combination with an agent other than a CCL2 antagonist; iii) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or iv) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In one embodiment, the patient's medical history is also reviewed in connection with the review of the patient's genotype.

In one embodiment, the selection of patient for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, is based upon the review of the patient's value for a parameter related to the patient's CCL2 expression level, the patient's genotype and the patient's medical history. In one embodiment, the patient is then treated, as described further.

### Method of Treating a Patient Having a CCR-2 Mediated Disorder.

The methods for treating a patient having a CCR-2 mediated disorder, e.g., a CCR-2 mediated disorder described herein, include a step of selecting a patient who over-expresses CCL2, or selecting a patient based upon a sequence present in the patient's CCL2 genomic sequence, and administering to the patient a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, to thereby treat the disorder.

In one embodiment, “selecting a patient” refers to the act or process of picking or choosing a patient based upon the patient's predisposition to be responsive to a treatment regimen. In one embodiment, the treatment regimen is an agent or compound which inhibits the interaction between CCR-2 and CCL2, e.g., an agent or compound which inhibits the interaction between CCR-2 and CCL2 as described herein. In another embodiment, the treatment regimen is a CCR-2 antagonist treatment regimen, e.g., a CCR-2 antagonist treatment regimen as described herein. In another embodiment, the treatment regimen is a CCL2 antagonist treatment regimen, e.g., a CCL2 antagonist treatment regimen as described herein. In one embodiment, the treatment regimen is a combination of any of the above listed treatment regimens.

A patient's predisposition to be responsive to a treatment regimen can be determined according to any of the evaluation methods described herein.

In one embodiment, a patient's predisposition to be responsive to a treatment regimen is based upon the patient's value for a parameter related to the patient's CCL2 expression level as compared to a reference standard. In one embodiment, the value may be determined according to any of the detection methods described herein. In one embodiment, a patient is considered predisposed to being responsive to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, when the patient's CCL2 expression level is over-expressed as compared to a reference standard for homozygous A individuals. Thus, when the test results indicate that a patient's CCL2 expression level is not significantly greater than the value of the reference standard for homozygous A individuals, the patient is not selected for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. However, such patients may be selected for treatment with one of the following treatment regimens: i) no treatment regimen; ii) a treatment regimen other than a CCR-2 antagonist ; iii) a treatment regimen other than a CCL2 antagonist; iv) a treatment regimen comprising a CCR-2 antagonist, e.g., a CCR-2 antagonist described herein, in combination with an agent other than a CCR-2 antagonist; v) a treatment regimen comprising a CCL2 antagonist, e.g., a CCL2 antagonist described herein, in combination with an agent other than a CCL2 antagonist; vi) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vii) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In one embodiment, when the CCR-2 mediated disorder is a cardiovascular disorder, for example, atherosclerosis, the treatment regimen other than a CCR-2 antagonist or other than a CCL2 antagonist is a treatment regimen using HMG-CoA reductase inhibitors, Statins, Fibrates, ACE inhibitors, anti-hypertensives, anti-thrombotics, β blockers, anti-diabetes drugs or anti-obesity drugs. In one embodiment, the HMG-CoA reductase inhibitor or Statin is selected from the group consisting of Atorvastatin, Cerivastatin, Fluvastatin, Lovastatin, Mevastatin, Pitavastatin, Pravastatin, Rosuvastatin, and Simvastatin.

When the test results indicate that a patient's CCL2 expression level is i) relatively the same as the reference standard for homozygous G or heterozygous individuals; or ii) over-expressed in comparison to the reference standard for homozygous G or heterozygous individuals, the patient is selected for treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. In one embodiment, the treatment regimen can include: i) an agent or compound which inhibits the interaction between CCR-2 and CCL2 as described herein; ii) a CCR-2 antagonist, e.g., a CCR-2 antagonist described herein; iii) a CCL2 antagonist e.g., a CCL2 antagonist described herein; iv) a CCR-2 antagonist, e.g., a CCR-2 antagonist described herein, in combination with an agent other than a CCR-2 antagonist; v) a CCL2 antagonist, e.g., a CCL2 antagonist described herein, in combination with an agent other than a CCL2 antagonist; vi) a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vii) a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In one embodiment, the patient's genotype in the patient's CCL2 genomic sequence is determined in connection with the determination of the patient's value for a parameter related to the patient's CCL2 expression level. In another embodiment, the patient's medical history is also reviewed in connection with the determination of the patient's value for a parameter related to the patient's CCL2 expression level.

In one embodiment, a patient's predisposition to be responsive to a treatment regimen is based upon the patient's genotype at a pre-selected nucleotide, allele or combination of alleles, or at a nucleotide, allele or combination of alleles in linkage disequilibrium with the pre-selected nucleotide, allele or combination of alleles, within the patient's CCL2 genomic sequence. In one embodiment, the patient's genotype is determined according to any of the diagnostic methods (e.g., genotyping or haplotyping methods) described herein. In one embodiment, the pre-selected nucleotide, allele or combination of alleles is/are within a non-coding region (e.g., an intron or a regulatory region) of the CCL2 genomic sequence. In one embodiment, the pre-selected nucleotide, allele or combination of alleles is a nucleotide at position 2236, 2485, 2936 or 5837 as numbered in SEQ ID NO:1, or one or more nucleotides, allele or combination of alleles in linkage disequilibrium with any of the above-listed nucleotides. In one embodiment, a patient is considered predisposed to being responsive to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, when the pre-selected nucleotide or allele in the patient's CCL2 genomic sequence at position 2485 is a G; when the pre-selected nucleotide or allele in the patient's CCL2 genomic sequence at position 2236 is an A; when the pre-selected nucleotide or allele in the patient's CCL2 genomic sequence at position 2936 is an A; or when the pre-selected nucleotide or allele in the patient's CCL2 genomic sequence at position 5837 is a C. In another embodiment, a patient is considered predisposed to being responsive to a treatment regimen comprising an agent or compound which inhibits the interaction between CCR-2 and CCL2; to a CCR-2 antagonist treatment regimen; to a CCL2 antagonist treatment regimen; or to a combination thereof, when the patient exhibits haplotype H1, as described in Table II above, in the patient's CCL2 genomic sequence.

In one embodiment, when the pre-selected nucleotide or allele in the patient's CCL2 genomic sequence is as described above (e.g., when a patient has a G allele present at the nucleotide at position 2485 as numbered in SEQ ID NO:1; or a nucleotide or allele in linkage disequilibrium with the nucleotide at position 2485; when a patient has an A allele present at the nucleotides at positions 2236 or 2936, as numbered in SEQ D NO:1, or nucleotides or alleles in linkage disequilibrium with the nucleotides at positions 2236 or 2936; when a patient has a C allele present at the nucleotide at position 5837, as numbered in SEQ ID NO:1, or a nucleotide or allele in linkage disequilibrium with the nucleotide at position 5837; or when the patient exhibits haplotype H1, as defined in Table II above), the patient is selected for treatment and is administered a first treatment regimen, e.g., an agent or compound which inhibits the interaction or association between CCR-2 and CCL2; a CCR-2 antagonist treatment regimen; a CCL2 antagonist treatment regimen; or a combination thereof. In one embodiment, the treatment regimen can include: i) an agent or compound which inhibits the interaction between CCR-2 and CCL2 as described herein; ii) a CCR-2 antagonist, e.g., a CCR-2 antagonist described herein; iii) a CCL2 antagonist e.g., a CCL2 antagonist described herein; iv) a CCR-2 antagonist, e.g., a CCR-2 antagonist described herein, in combination with an agent other than a CCR-2 antagonist; v) a CCL2 antagonist, e.g., a CCL2 antagonist described herein, in combination with an agent other than a CCL2 antagonist; vi) a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, or vii) a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In another embodiment, when the pre-selected nucleotide in the patient's CCL2 genomic sequence at position 2485, as numbered in SEQ ID NO:1, is homozygous A, or a nucleotide or allele in linkage disequilibrium with the nucleotide or allele at position 2485, the patient is selected for treatment with a second treatment regimen, which is different from the first treatment regimen, e.g., the patient is administered a treatment regimen other than an agent or compound which inhibits the interaction between CCR-2 and CCL2; a treatment regimen other than a CCR-2 antagonist; or a treatment regimen other than a CCL2 antagonist. This other treatment regimen may be selected from the group of treatment regimens consisting of: i) no treatment regimen; ii) a treatment regimen other than a CCR-2 antagonist; iii) a treatment regimen other than a CCL2 antagonist; iv) a treatment regimen comprising a CCR-2 antagonist, e.g., a CCR-2 antagonist described herein, in combination with an agent other than a CCR-2 antagonist; v) a treatment regimen comprising a CCL2 antagonist, e.g., a CCL2 antagonist described herein, in combination with an agent other than a CCL2 antagonist; vi) a treatment regimen comprising a CCR-2 antagonist or CCL2 antagonist, e.g., a CCR-2 antagonist or CCL2 antagonist described herein, which is administered at a decreased dose and/or a decreased administration schedule, e.g., at a dose statistically less than recommended for patients having the disorder and/or at a dosing frequency less than recommended for patients having the disorder, and vii) a treatment regimen comprising a CCR-2 antagonist or CCL2 antagonist, e.g., a CCR-2 antagonist or CCL2 antagonist described herein, which is administered at an increased dose and/or an increased administration schedule, e.g., at a dose statistically greater than recommended for patients having the disorder and/or at a dosing frequency greater than recommended for patients having the disorder.

In one embodiment, when the CCR-2 mediated disorder is a cardiovascular disorder, for example, atherosclerosis, the treatment regimen other than a CCR-2 antagonist or other than a CCL2 antagonist is a treatment regimen using HMG-CoA reductase inhibitors, Statins, Fibrates, ACE inhibitors, anti-hypertensives, anti-thrombotics, β blockers, anti-diabetes drugs or anti-obesity drugs. In one embodiment, the HMG-CoA reductase inhibitor or Statin is selected from the group consisting of Atorvastatin, Cerivastatin, Fluvastatin, Lovastatin, Mevastatin, Pitavastatin, Pravastatin, Rosuvastatin, and Simvastatin.

In one embodiment, the patient's medical history is also reviewed in connection with the determination of the patient's genotype.

In one embodiment, the selection of a patient is based upon the determination of the patient's value for a parameter related to the patient's CCL2 expression level, the patient's genotype and a review of the patient's medical history.

In one embodiment of the method, once a patient has been selected, the patient is then administered an agent or compound which inhibits the interaction between CCR-2 and CCL2, or the patient is administered a CCR-2 antagonist, a CCL2 antagonist, or a combination thereof.

Examples of CCR-2 antagonists include peptides, polypeptides (e.g., soluble fragments of ligands, non-naturally occurring peptides that bind CCR-2 (e.g., peptides obtained by phage display)), antibodies or fragments thereof, and non-protein molecules (e.g. small molecules). Examples of CCR-2 antagonists can be found, for example, in the patent applications listed below in Table III.

TABLE III

| | Patent Number(s) or | | Patent Number(s) or |
|---|---|---|---|
| PCT Publication | Patent Publication | PCT Publication | Patent Publication |
| Number | Number(s) (US or other) | Number | Number(s) (US or other) |
| WO 06/067401 | N/A | WO 05/070133 | N/A |
| WO 05/118579 | N/A | WO 05/067502 | N/A |
| WO 05/1178905 | N/A | WO 05/044795 | N/A |
| WO 01/051467 | U.S. Pat. No. 6984657 | WO 05/044264 | N/A |
| WO 01/051466 | US2003144339 | WO 05/014537 | N/A |
| WO 00/046197 | U.S. Pat. No. 6613760 | WO 05/010154 | US2006183731 |
| WO 00/046196 | U.S. Pat. No. 6737435 | WO 04/110376 | N/A |
| WO 00/046195 | US2005026975 | WO 04/094371 | N/A |
| | U.S. Pat. No. 6911465 | | |
| WO 00/046198 | U.S. Pat. No. 6569888 | WO 04/092124 | US2006069088 |
| WO 00/046199 | U.S. Pat. No. 6833387 | WO 04/082616 | US2006178363 |
| WO 99/07678 | U.S. Pat. No. 6288103 | WO 04/082682 | N/A |
| WO 99/40913 | U.S. Pat. No. 6291507 | WO 04/076411 | N/A |
| WO 99/40914 | U.S. Pat. No. 6479527 | WO 04/042351 | US 05/250814 |
| WO 05/094798 | US2005260139 | WO 04/041279 | US2005261325 |
| WO 06/016152 | N/A | WO 04/041163 | US2006173013 |
| WO 06/076644 | US2006173019 | WO 04/041161 | US2006116421 |
| N/A | JP 2001278886 | WO 04/041777 | US2005250781 |
| WO 01/057044 | N/A | WO 03/093266 | US2005107422 |
| | | | US2005101628 |
| WO 02/079190 | N/A | WO 03/093231 | US2006089379 |
| WO 02/079151 | N/A | WO 03/092586 | US2004167156 |
| | | | U.S. Pat. No. 6812234 |
| | | | US2005101628 |
| | | | US2006030582 |
| WO 06/073592 | US 06/0111404 | WO 02/013824 | US2002049222 |
| | | | U.S. Pat. No. 6545023 |
| WO 06/013427 | N/A | WO 05/077932 | N/A |
| WO 06/004684 | US2006004018 | WO 04/092169 | N/A |
| WO 06/004741 | US2006020133 | WO 04/094424 | N/A |
| WO 05/115392 | US2005267146 | WO 04/014847 | US2005171163 |
| | | | U.S. Pat. No. 7067538 |
| WO 05/101838 | US2005261310 | WO 02/070523 | US2003008893 |
| | | | U.S. Pat. No. 6821964 |
| | | | US2005234090 |
| WO 05/060665 | US2005192302 | N/A | US2002028833 |
| | | | U.S. Pat. No. 6458806 |
| WO 04/050024 | N/A | N/A | U.S. Pat. No. 6011052 |
| WO 06/036527 | US2006069123 | WO 98/06703 | U.S. Pat. No. 6184235 |
| | | | U.S. Pat. No. 6348487 |
| | | | US2002099054 |
| | | | U.S. Pat. No. 6534521 |
| WO 06/015986 | N/A | WO 06/042954 | N/A |
| WO 06/012135 | N/A | WO 06/016039 | N/A |
| WO 05/118578 | N/A | WO 04/096798 | US2006135575 |
| WO 05/118574 | N/A | WO 03/014110 | US2004235822 |
| WO 04/069810 | US2006058289 | WO 03/014105 | US2004259876 |
| | | | US2005107606 |
| WO 04/069809 | US2006058289 | WO 03/089004 | US2005154016 |
| WO 06/088813 | N/A | WO 99/32468 | U.S. Pat. No. 6166006 |
| | | | U.S. Pat. No. 6413947 |
| WO 06/074265 | N/A | WO 00/069432 | N/A |
| WO 06/001958 | N/A | WO 9744329 | U.S. Pat. No. 6686353 |
| WO 05/120505 | N/A | N/A | JP 2000229856 |
| WO 05/110409 | N/A | WO 03/030897 | US2003149081 |
| | | | U.S. Pat. No. 6727275 |
| | | | US2004157914 |
| | | | U.S. Pat. No. 6936633 |
| | | | US2005004180 |
| WO 05/105092 | N/A | WO 00/005265 | U.S. Pat. No. 6352832 |
| | | | U.S. Pat. No. 6312689 |
| | | | U.S. Pat. No. 6458353 |
| | | | U.S. Pat. No. 6491915 |
| | | | U.S. Pat. No. 6406694 |
| | | | U.S. Pat. No. 6406865 |
| | | | U.S. Pat. No. 6448021 |
| | | | U.S. Pat. No. 6451522 |
| | | | U.S. Pat. No. 6395497 |
| | | | US20050048052 |
| | | | US20040265303 |
| WO 05/080371 | N/A | WO 01/057226 | U.S. Pat. No. 6727349 |
| | | | U.S. Pat. No. 6696550 |
| | | | US20040132980 |
| | | | US20040126851 |
| WO 05/072361 | N/A | N/A | U.S. Pat. No. 7053202 |
| | | | US20060147445 |
| | | | US20040151721 |
| WO 97/31949 | U.S. Pat. No. 6084075 | | |

Likewise, examples of CCL2 antagonists include peptides, polypeptides (e.g., soluble fragments of binding partners, e.g., CCR-2, non-naturally occurring peptides that bind CCL2 (e.g., peptides obtained by phage display)), antibodies or fragments thereof, and non-protein molecules (e.g. small molecules). Examples of CCL2 antagonists can be found, for example, in the patent applications listed below in Table IV.

TABLE IV

| | Patent Number(s) | | Patent Number(s) |
|---|---|---|---|
| | or Patent | | or Patent |
| PCT | Publication | | Publication |
| Publication | Number(s) | PCT Publication | Number(s) |
| Number | (US or other) | Number | (US or other) |
| WO 99/05279 | U.S. Pat. No. | N/A | JP2003137812 |
| | 6383782 | | |
| WO 04/050836 | N/A | N/A | JP 2002284698 |
| WO 03/048083 | US2005025768 | N/A | JP2005281240 |
| WO 04/096850 | US2005148507 | N/A | JP 1997067399 |
| WO 04/024088 | US2004197303 | WO 02/02640 | US2004047860 |
| | U.S. Pat. No. | | |
| | 7091310 | | |
| WO 04/024921 | N/A | WO 05/037305 | N/A |
| WO 06/085961 | US20060039913 | WO 05/063812 | N/A |
| WO 05/044200 | US2005232923 | WO 03/084993 | N/A |
| WO 03/083059 | N/A | WO 01/089582 | US2003162737 |
| WO 03/037376 | US2005222019 | N/A | CA 2152141 |

Likewise, examples of agents or compounds which inhibit the interaction between CCR-2 and CCL2 include peptides, polypeptides (e.g., soluble fragments of binding partners, e.g., CCR-2 or CCL2, non-naturally occurring peptides that bind CCL2 or CCR-2 (e.g., peptides obtained by phage display)), antibodies or fragments thereof, and non-protein molecules (e.g. small molecules). Examples of agents or compounds which inhibit the interaction between CCR-2 and CCL2 can be found, for example, in the patent applications listed below in Table V.

TABLE V

| | Patent Number(s) or | | Patent Number(s) or |
|---|---|---|---|
| | Patent Publication | | Patent Publication |
| | Number(s) (US or | PCT Publication | Number(s) (US or |
| PCT Publication Number | other) | Number | other) |
| WO 05/101002 | N/A | WO 02/050019 | US2003060459 |
| | | | U.S. Pat. No. 6974836 |
| | | | US2005282882 |
| WO 05/079519 | US2005192276 | WO 05/084667 | US2005234034 |
| | | | US2006074121 |
| WO 05/079496 | US2005227960 | WO 06/058201 | US2006128702 |
| WO 05/021500 | US2005054627 | WO 05/014600 | |
| WO 05/021499 | US2005065147 | WO 02/081463 | US2003092728 |
| | | | U.S. Pat. No. 6677365 |
| | | | US2004127513 |
| | | | U.S. Pat. No. 6998407 |
| WO 05/020899 | US2005054626 | WO 02/070509 | US2003105085 |
| | | | U.S. Pat. No. 6809113 |
| | | | US2005054668 |
| WO 05/021498 | US2005043392 | WO 02/060900 | US2003096705 |
| | | | U.S. Pat. No. 6670364 |
| | | | US2004077680 |
| | | | U.S. Pat. No. 6992086 |
| | | | US2004198719 |
| | | | U.S. Pat. No. 6962926 |
| WO 04/098512 | US2004235835 | WO 05/112914 | US2006020038 |
| WO 04/098516 | US2004235836 | WO 04/007423 | US2004082807 |
| | | | U.S. Pat. No. 6960683 |
| | | | US2006079713 |
| WO 04/071449 | US2004186143 | N/A | US2004138171 |
| | | | U.S. Pat. No. 7015202 |
| | | | US2006116337 |
| WO 04/071460 | US2004186140 | JP 2000297098 | N/A |
| WO 03/075853 | US2003216434 | JP2005187451 | N/A |
| | U.S. Pat. No. 7087604 | | |
| | US2006135503 | | |
| WO 02/060859 | US2003004151 | N/A | JP11060502 |
| | U.S. Pat. No. 6706712 | | |
| | US2004110736 | | |
| | U.S. Pat. No. 7045521 | | |
| | US2006135502 | | |

In one embodiment, a combination of therapeutic regimens as described herein, refers to, for example, an agent or compound which inhibits the interaction between CCR-2 and CCL2 (e.g., any of the agents or compounds which inhibit the interaction between CCR-2 and CCL2 in any of the patent applications listed in Table V) administered in combination with an anti-CCL2 antagonistic antibody (e.g., any anti-CCL2 antibody in any of the patent applications listed in Table IV, e.g., 1A1; 4N4; 5A13; 6D21; 6I5; 7H1; 11K2; D9; 1M11; 3N10; 5J23; 7F7; 6B11; 9B11; 9C11; 10D18; 12F15; AAV293; AAV294; C588; C750; C751; or ABN912, as described herein).

In another embodiment, a combination of therapeutic regimens as described herein, refers to, for example, an agent or compound which inhibits the interaction between CCR-2 and CCL2 (e.g., any of the agents or compounds which inhibit the interaction between CCR-2 and CCL2 in any of the patent applications listed in Table V) administered in combination with an anti-CCR-2 antagonistic antibody (e.g., any anti-CCR-2 antibody in any of the patent applications listed in Table III, e.g., MLN1202; 1D9; 8G2; MCPR-04; MCPR-05; or MCPR-06, as described herein).

In another embodiment, a combination of therapeutic regimens as described herein, refers to, for example, an agent or compound which inhibits the interaction between CCR-2 and CCL2 (e.g., any of the agents or compounds which inhibit the interaction between CCR-2 and CCL2 in any of the patent applications listed in Table V) administered in combination with a non-protein CCR-2 antagonist (e.g., any of the non-protein CCR-2 antagonists in any of the patent applications listed in Table III).

In another embodiment, a combination of therapeutic regimens as described herein, refers to, for example, an agent or compound which inhibits the interaction between CCR-2 and CCL2 (e.g., any of the agents or compounds which inhibit the interaction between CCR-2 and CCL2 in any of the patent applications listed in Table V) administered in combination with a non-protein CCL2 antagonist (e.g., any of the non-protein CCL2 antagonists in any of the patent applications listed in Table IV).

In another embodiment, a combination of therapeutic regimens as described herein, refers to, for example, an agent or compound which inhibits the interaction between CCR-2 and CCL2 (e.g., any of the agents or compounds which inhibit the interaction between CCR-2 and CCL2 in any of the patent applications listed in Table V) administered in combination with another agent or compound which inhibits the interaction between CCR-2 and CCL2 (e.g., any of the agents or compounds which inhibit the interaction between CCR-2 and CCL2 in any of the patent applications listed in Table V).

In another embodiment, a combination of therapeutic regimens as described herein, refers to, for example, a non-protein CCL2 antagonist (e.g., any of the non-protein CCL2 antagonists in any of the patent applications listed in Table IV) administered in combination with a non-protein CCR-2 antagonist (e.g., any of the non-protein CCR-2 antagonists in any of the patent applications listed in Table III).

In another embodiment, a combination of therapeutic regimens as described herein, refers to, for example, a non-protein CCL2 antagonist (e.g., any of the non-protein CCL2 antagonists in any of the patent applications listed in Table IV) administered in combination with another non-protein CCL2 antagonist (e.g., any of the non-protein CCL2 antagonists in any of the patent applications listed in Table IV).

In another embodiment, a combination of therapeutic regimens as described herein, refers to, for example, a non-protein CCL2 antagonist (e.g., any of the non-protein CCL2 antagonists in any of the patent applications listed in Table IV) administered in combination with an anti-CCL2 antagonistic antibody (e.g., any anti-CCL2 antibody in any of the patent applications listed in Table IV, e.g., 1A1; 4N4; 5A13; 6D21; 6I5; 7H1; 11K2; D9; 1M11; 3N10; 5J23; 7F7; 6B11; 9B11; 9C11; 10D18; 12F15; AAV293; AAV294; C588; C750; C751; or ABN912, as described herein).

In another embodiment, a combination of therapeutic regimens as described herein, refers to, for example, a non-protein CCL2 antagonist (e.g., any of the non-protein CCL2 antagonists in any of the patent applications listed in Table IV) administered in combination with an anti-CCR-2 antagonistic antibody (e.g., any anti-CCR-2 antibody in any of the patent applications listed in Table III, e.g., MLN1202; 1D9; 8G2; MCPR-04; MCPR-05; or MCPR-06, as described herein).

In another embodiment, a combination of therapeutic regimens as described herein, refers to, for example, a non-protein CCR-2 antagonist (e.g., any of the non-protein CCR-2 antagonists in any of the patent applications listed in Table III) administered in combination with another non-protein CCR-2 antagonist (e.g., any of the non-protein CCR-2 antagonists in any of the patent applications listed in Table III).

In another embodiment, a combination of therapeutic regimens as described herein, refers to, for example, a non-protein CCR-2 antagonist (e.g., any of the non-protein CCR-2 antagonists in any of the patent applications listed in Table III) administered in combination with an anti-CCL2 antagonistic antibody (e.g., any anti-CCL2 antibody in any of the patent applications listed in Table IV, e.g., 1A1; 4N4; 5A13; 6D21; 6I5; 7H1; 11K2; D9; 1M11; 3N10; 5J23; 7F7; 6B11; 9B11; 9C11; 10D18; 12F15; AAV293; AAV294; C588; C750; C751; or ABN912, as described herein).

In another embodiment, a combination of therapeutic regimens as described herein, refers to, for example, a non-protein CCR-2 antagonist (e.g., any of the non-protein CCR-2 antagonists in any of the patent applications listed in Table III) administered in combination with an anti-CCR-2 antagonistic antibody (e.g., any anti-CCR-2 antibody in any of the patent applications listed in Table II, e.g., MLN1202; 1D9; 8G2; MCPR-04; MCPR-05; or MCPR-06, as described herein).

In another embodiment, a combination of therapeutic regimens as described herein, refers to, for example, an anti-CCL2 antagonistic antibody (e.g., any anti-CCL2 antibody in any of the patent applications listed in Table IV, e.g., 1A1; 4N4; 5A13; 6D21; 6I5; 7H1; 11K2; D9; 1M11; 3N10; 5J23; 7F7; 6B11; 9B11; 9C11; 10D18; 12F15; AAV293; AAV294; C588; C750; C751; or ABN912, as described herein) administered in combination with an anti-CCR-2 antagonistic antibody (e.g., any anti-CCR-2 antibody in any of the patent applications listed in Table III, e.g., MLN1202; 1D9; 8G2; MCPR-04; MCPR-05; or MCPR-06, as described herein).

### Non-Protein Molecules

The present invention relates to the use of CCR-2 antagonists or CCL2 antagonists, such as the non-protein molecules described in the patent applications listed in Tables III-V above, for treatment of CCR-2 mediated disorders as defined herein. The non-protein molecule compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al. (1994) J. Med. Chem. 37:2678-85); spatially addressable parallel solid phase or solution phase libraries; synthetic library methods requiring deconvolution; the “one-bead one-compound” library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA. 90:6909-13; Erb et al. (1994) Proc. Natl. Acad. Sci. USA 91:11422-426; Zuckermann et al. (1994). J. Med. Chem. 37:2678-85; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233-51.

In one embodiment, the non-protein molecule is selected from the group consisting of MK0812, INCB3284, SSR 150106, CCX915, INCB3344 and AGI-1096.

In one embodiment, the non-protein molecule is selected from the molecules described in US2006/0111404 and WO06/073592 (both of which are listed in Table III). In one embodiment, the non-protein molecule is N-[2-({(3R)-1-[trans-4-hydroxy-4-(6-methoxypyridin-3-yl)-cyclohexyl]pyrrolidin-3-yl}amino)-2-oxoethyl]-3-(trifluoromethyl)-benzamide. In another embodiment, the non-protein molecule is the molecule depicted in structure I.

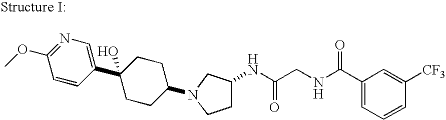

In one embodiment, the non-protein molecule is selected from the molecules described in WO 05/044795 and WO 05/044264 (both of which are listed in Table III). In one embodiment, the non-protein molecule is ((1R,3S)-3-isopropyl-3-{[3-(trifluoromethyl)-7,8-dihydro-1,6-naphthyridin-6(5H)-yl]carbonyl)cyclopentyl)[(3S,4S)-3-methoxytetrahydro-2H-pyran-4-yl]amine (see structure II) and its succinate salt (see structure III). In another embodiment, the non-protein molecule is the molecule depicted in structure II. In another embodiment, the non-protein molecule is the molecule depicted in structure III.

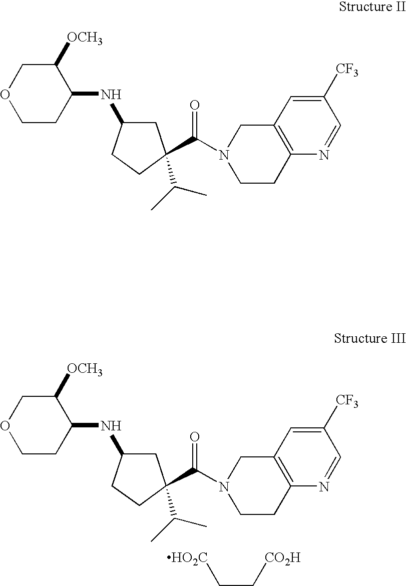

### Antibodies

The present invention relates to the use of CCR-2 antagonists or CCL2 antagonists, such as the antibodies described in the patent applications listed in Tables III-V above, for treatment of CCR-2 mediated disorders as defined herein.

The term “antibody” as used herein refers to an immunoglobulin molecule or immunologically active portion thereof, i.e., an antigen-binding portion. Examples of immunologically active portions of immunoglobulin molecules include scFV and dcFV fragments, Fab and F(ab′)₂ fragments which can be generated by treating the antibody with an enzyme such as papain or pepsin, respectively.

The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a detectable agent, a toxin or an imaging agent.

The present invention relates to the use of CCR-2 antagonists, such as anti-CCR-2 antibodies or functional fragments thereof which bind mammalian CCR-2 or a portion of CCR-2, for treatment or diagnosis of CCR-2 mediated disorders as defined herein. In one embodiment, the antibody has specificity for mammalian CCR-2 or portion thereof. In another embodiment, the antibody has specificity for human CCR-2 or portion thereof. In one embodiment, the antibodies (immunoglobulins) are raised against an isolated and/or recombinant mammalian CCR-2 or portion thereof (e.g., peptide) or against a host cell which expresses mammalian CCR-2. In a preferred embodiment, the antibodies specifically bind human CCR-2 receptor(s) (e.g., CCR-2α and/or CCR-2β) or a portion thereof, and in a particularly preferred embodiment the antibodies have specificity for a naturally occurring or endogenous human CCR-2. Antibodies or functional fragments thereof which can inhibit one or more functions characteristic of a mammalian CCR-2, such as a binding activity (e.g., ligand, inhibitor and/or promoter binding), a signaling activity (e.g., activation of a mammalian G protein, induction of a rapid and transient increase in the concentration of cytosolic free calcium [Ca²⁺]i), and/or stimulation of a cellular response (e.g., stimulation of chemotaxis, exocytosis or inflammatory mediator release by leukocytes, integrin activation) are also encompassed by the present invention, such as an antibody which can inhibit binding of a ligand (i.e., one or more ligands) to CCR-2 and/or one or more functions mediated by CCR-2 in response to a ligand. For example, in one aspect, the antibodies or functional fragments thereof can inhibit (reduce or prevent) the interaction of receptor with a natural ligand, such as MCP-1 (CCL2), MCP-2 (CCL8), MCP-3 (CCL7), MCP-4 (CCL13), HCC4 (CCL16) and/or HIV. In another aspect, an antibody or functional fragment thereof that binds to CCR-2 can inhibit binding of CCL2, CCL8, CCL7, CCL13, CCL16 and/or HIV to mammalian CCR-2 (e.g., human CCR-2, non-human primate CCR-2, murine CCR-2). The antibodies or functional fragments thereof of the present invention can inhibit functions mediated by human CCR-2, including leukocyte trafficking, HIV entry into a cell, T cell activation, inflammatory mediator release and/or leukocyte degranulation. Preferably, the antibodies or fragments can bind CCR-2 with an affinity of at least about 0.1×10⁻⁹ M, preferably at least about 1×10⁻⁹ M, and more preferably at least about 3×10⁻⁹ M. In a particular embodiment, antibodies or functional fragments thereof demonstrate inhibition of chemokine-induced (e.g., MCP-1-induced) chemotaxis of cells (e.g., PBMC) at less than about 150 μg/ml, preferably less than about 100 μg/ml, more preferably less than about 50 μg/ml, and even more preferably less than about 20 μg/ml.

The present invention also relates to the use of antibodies or functional fragments thereof which bind to one or more of the CCR-2 natural ligands (e.g. MCP-1 (CCL2), MCP-2 (CCL8), MCP-3 (CCL7), MCP-4 (CCL13), and/or HCC4 (CCL16)) for the treatment or diagnosis of CCR-2 mediated disorders as defined herein. In a preferred embodiment, the invention relates to the use of CCL2 antagonists, such as anti-CCL2 antibodies or functional fragments thereof for the treatment of CCR-2 mediated disorders as defined herein. In one embodiment, the antibody has specificity for mammalian CCL2 or portions thereof. In one embodiment, the antibody has specificity for human CCL2 or portions thereof. In one embodiment, the antibodies (immunoglobulins) are raised against an isolated and/or recombinant mammalian CCL2 or portions thereof (e.g., peptide) or against a host cell which expresses mammalian CCL2. In a preferred embodiment, the antibodies specifically bind human CCL2 or a portion thereof, and in a preferred embodiment the antibodies have specificity for a naturally occurring or endogenous human CCL2. Antibodies or functional fragments thereof which can inhibit one or more functions characteristic of a mammalian CCL2, such as a binding to its receptor (i.e. CCR-2) and inducing a signaling activity, e.g., activation of a mammalian G protein, induction of a rapid and transient increase in the concentration of cytosolic free calcium [Ca²⁺]i, and/or stimulation of a cellular response (e.g., stimulation of chemotaxis, exocytosis or inflammatory mediator release by leukocytes, integrin activation) are also encompassed by the present invention. Such antibodies include antibodies which can inhibit (reduce or prevent) the interaction between CCR-2 and CCL2, and inhibit one or more functions mediated by CCR-2 in response to a ligand. The antibodies or functional fragments thereof of the present invention can inhibit functions mediated by human CCR-2, including leukocyte trafficking, HIV entry into a cell, T cell activation, inflammatory mediator release and/or leukocyte degranulation. Preferably, the antibodies or fragments can bind CCL2 with an affinity of at least about 0.1×10⁻⁹ M, preferably at least about 1×10⁻⁹ M, and more preferably at least about 3×10⁻⁹ M. In a particular embodiment, antibodies or functional fragments thereof demonstrate inhibition of chemokine-induced (e.g., CCL2-induced) chemotaxis of cells (e.g., PBMC) at less than about 150 μg/ml, preferably less than about 100 μg/ml, more preferably less than about 50 μg/ml, and even more preferably less than about 20 μg/ml.

In a further embodiment of the invention, the antibodies or functional fragments thereof of the invention can inhibit binding of a CCR-2 ligand (e.g., CCL2, CCL8, CCL7, CCL13, CCL16 and/or HIV) to CCR-2 with an IC₅₀ of less than about 1.0 μg/ml, preferably less than about 0.05 μg/ml, and more preferably less than about 0.005 μg/ml.

In one embodiment the antibody can bind to the extracellular portion of the CCR-2, CCL2, CCL8, CCL7, CCL13 or CCL16 proteins, e.g., it can bind to a whole cell which expresses the CCR-2, CCL2, CCL8, CCL7, CCL13 or CCL16 proteins. In another embodiment, the antibody can bind an intracellular portion of the CCR-2, CCL2, CCL8, CCL7, CCL13 or CCL16 proteins.

A skilled artisan understands that methods described herein which use an antibody can also utilize functional fragments (e.g., antigen-binding fragments) of the antibody which have the same or similar epitopic specificity as the antibody, and combinations thereof, optionally in combination with antibodies or fragments having an epitopic specificity which is not the same as or similar to the first antibody. Antibodies used in the present invention can be raised against an appropriate immunogen, such as isolated and/or recombinant mammalian CCR-2, CCL2, CCL8, CCL7, CCL13 or CCL16 proteins or portions thereof, or synthetic molecules, such as synthetic peptides. The immunogen should include at least 8 amino acid residues of the target molecule, e.g., CCR-2 and encompasses an epitope of the target molecule, e.g., CCR-2. Preferably, the immunogen includes at least 10 amino acid residues, more preferably at least 15 amino acid residues, even more preferably at least 20 amino acid residues, and most preferably at least 30 amino acid residues. In some embodiments, the immunogen includes less than 1000 amino acids, 500 amino acids, 100 amino acids, 50 amino acids. In a preferred embodiment, cells which express the CCR-2, CCL2, CCL8, CCL7, CCL13 or CCL16 proteins, such as transfected cells, can be used as immunogens or in a screen for antibody which binds the protein.

The antibodies used in the present invention, and fragments thereof, are useful in therapeutic, diagnostic and research applications as described herein. The present invention encompasses use of an antibody or functional portion thereof for use in therapy (including prophylaxis) or diagnosis (e.g., evaluation of an individual's propensity to be responsive to a treatment regimen, e.g., a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof; evaluation of a subject's propensity for developing particular diseases or conditions as described herein), and use of such antibodies or functional portions thereof for the manufacture of a medicament for use in treatment of diseases or conditions as described herein.

Preparation of immunizing antigen, and polyclonal and monoclonal antibody production can be performed as described herein, or using other suitable techniques. A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991)). Generally, a hybridoma can be produced by fusing a suitable immortal cell line (e.g., a myeloma cell line such as SP2/0) with antibody producing cells. The antibody producing cell, preferably those of the spleen or lymph nodes, are obtained from animals immunized with the antigen of interest. The fused cells (hybridomas) can be isolated using selective culture conditions, and cloned by limiting dilution. Cells which produce antibodies with the desired binding properties can be selected by a suitable assay (e.g., ELISA). Immunoglobulins of nonhuman origin having binding specificity for CCR-2, CCL2, CCL8, CCL7, CCL13 or CCL16 can also be obtained from antibody libraries (e.g., a phage library comprising nonhuman Fab molecules).

Other suitable methods of producing or isolating antibodies which bind CCR-2, CCL2, CCL8, CCL7, CCL13 or CCL16, including completely human or artificial antibodies, can be used, including, for example, methods which select recombinant antibody (e.g., single chain Fv or Fab) from a library, or which rely upon immunization of transgenic animals (e.g., mice) capable of producing a repertoire of human antibodies (see e.g., Jakobovits et al., Proc. Natl. Acad. Sci. USA, 90: 2551-2555 (1993); Jakobovits et al., Nature, 362: 255-258 (1993); Lonberg et al., U.S. Pat. No. 5,545,806; Surani et al., U.S. Pat. No. 5,545,807).

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93. For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al. (1994) Bio/technology 12:899-903).

Single chain antibodies, and chimeric, humanized or primatized (CDR-grafted) antibodies, as well as chimeric or CDR-grafted single chain antibodies, and the like, comprising portions derived from different species, are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; and Queen et al., U.S. Pat. Nos. 5,585,089, 5,698,761 and 5,698,762. See also, Newman, R. et al. (1992) BioTechnology 10: 1455-1460, regarding primatized antibody, and Ladner et al., U.S. Pat. No. 4,946,778 and Bird, R. E. et al. (1988) Scienc, 242: 423-426) regarding single chain antibodies.

In addition, functional fragments of antibodies, including fragments of chimeric, humanized, primatized or single chain antibodies, can also be produced. Functional fragments of the foregoing antibodies retain at least one binding function and/or modulation function of the full-length antibody from which they are derived. Preferred functional fragments retain an antigen-binding function of a corresponding full-length antibody (e.g., the ability to bind a mammalian CCR-2, CCL2, CCL8, CCL7, CCL13 or CCL16). Particularly preferred functional fragments retain the ability to inhibit one or more functions characteristic of a mammalian CCR-2, CCL2, CCL8, CCL7, CCL13 or CCL16, such as a binding activity, a signaling activity, and/or stimulation of a cellular response. For example, in one embodiment, a functional fragment can inhibit the interaction of CCR-2 with one or more of its ligands (e.g., CCL2, CCL8, CCL7, CCL13 and/or CCL16) and/or can inhibit one or more receptor-mediated functions, such as leukocyte trafficking, HIV entry into cells, T cell activation, inflammatory mediator release and/or leukocyte degranulation.

For example, antibody fragments capable of binding to a mammalian CCR-2 receptor or to a mammalian CCL2, CCL8, CCL7, CCL13 or CCL16 chemokine or portions thereof, including, but not limited to, Fv, Fab, Fab′ and F(ab′)₂ fragments are encompassed by the invention. Such fragments can be produced by enzymatic cleavage or by recombinant techniques, for example. For instance, papain or pepsin cleavage can generate Fab or F(ab′)₂ fragments, respectively. Antibodies can also be produced in a variety of truncated forms using antibody genes in which one or more stop codons has been introduced upstream of the natural stop site. For example, a chimeric gene encoding a F(ab′)₂ heavy chain portion can be designed to include DNA sequences encoding the CH₁ domain and hinge region of the heavy chain.

A humanized or complementarity determining region (CDR)-grafted antibody will have at least one or two, but generally all three recipient CDR's (of heavy and or light immunoglobulin chains) replaced with a donor CDR. The antibody may be replaced with at least a portion of a non-human CDR or only some of the CDR's may be replaced with non-human CDR's. It is only necessary to replace the number of CDR's required for binding of the humanized antibody to the target molecule, e.g., CCR-2 or a fragment thereof. Preferably, the donor will be a rodent antibody, e.g., a rat or mouse antibody, and the recipient will be a human framework or a human consensus framework. Typically, the immunoglobulin providing the CDR's is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, (1987) From Genes to Clones (Verlagsgesellschaft, Weinheim, Germany). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison (1985) Science 229:1202-1207, by Oi et al. (1986) BioTechniques 4:214, and by Queen et al. U.S. Pat. Nos. 5,585,089, 5,693,761 and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a 9136 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDR's of an immunoglobulin chain can be replaced. See e.g., U.S. Pat. No. 5,225,539; Jones et al. (1986) Nature 321:552-525; Verhoeyan et al. (1988) Science 239:1534; Beidler et al. (1988) J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

An antibody (or fragment thereof) may be conjugated to a therapeutic moiety such as a cytotoxin, a therapeutic agent or a radioactive ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids). Radioactive ions include, but are not limited to iodine, yttrium and praseodymium.

The conjugates of the invention can be used for modifying a given biological response, the therapeutic moiety is not to be construed as limited to classical chemical therapeutic agents. For example, the therapeutic moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Alternatively, an antibody can be conjugated to a second antibody to form an antibody heteroconjugate as described by Segal in U.S. Pat. No. 4,676,980.

The antibodies used in the present invention can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

In an exemplary embodiment, the antibody is MLN1202. “MLN1202” is a humanized monoclonal antibody created, in part, by the insertion of the CDRs from an immunoglobulin 2a (IgG2a) mouse monoclonal antibody (mulD9) into a human IgG1 immunoglobulin framework. MLN1202 is a potent and specific antagonist of CCR-2. The MLN1202 humanized variable light chain is shown in SEQ ID NO:2 and is encoded by the nucleic acid sequence shown in SEQ ID NO:6. The MLN1202 humanized variable heavy chain is shown in SEQ ID NO:3 and is encoded by the nucleic acid sequence shown in SEQ ID NO:7. The MLN1202 heavy chain constant region is shown in SEQ ID NO:4 and is encoded by the nucleic acid sequence shown in SEQ ID NO:8. The MLN1202 light chain constant region is shown in SEQ ID NO:5 and is encoded by the nucleic acid sequence shown in SEQ ID NO:9 (see, e.g., U.S. Pat. No. 6,727,349; U.S. Pat. No. 6,696,550; US2004/0132980; US2004/0126851; U.S. Pat. No. 6,312,689; US2005/0048052; US2004/0265303; U.S. Pat. No. 7,053,202; US2006/0147445; and US2004/0151721 incorporated herein by reference).

In another embodiment, the antibody is murine monoclonal antibody 1D9. The hybridoma cell line producing 1D9 was deposited on Jul. 17, 1998, on behalf of LeukoSite, Inc., 215 First Street, Cambridge, Mass. 02142, U.S.A. (now Millennium Pharmaceuticals, Inc., 40 Landsdowne Street, Cambridge, Mass. 02139, U.S.A.), at the American Type Culture Collection, 10801 University Boulevard, Manassas, Va. 20110, U.S.A., under Accession Nos. HB-12549. In another embodiment, the antibody is murine monoclonal antibody 8G2. The hybridoma cell line producing 8G2 was deposited on Jul. 17, 1998, on behalf of LeukoSite, Inc., 215 First Street, Cambridge, Mass. 02142, U.S.A. (now Millennium Pharmaceuticals, Inc., 40 Landsdowne Street, Cambridge, Mass. 02139, U.S.A.), at the American Type Culture Collection, 10801 University Boulevard, Manassas, Va. 20110, U.S.A., under Accession Nos. HB-12550 (see, e.g., U.S. Pat. No. 6,312,689; U.S. Pat. No. 6,458,353; U.S. Pat. No. 6,491,915; U.S. Pat. No. 6,406,694; U.S. Pat. No. 6,727,349; U.S. Pat. No. 6,696,550; and U.S. Pat. No. 7,053,202, incorporated herein by reference).

In another embodiment, the antibody is MCPR-04, MCPR-05 or MCPR-06, which are anti-CCR-2 antagonistic monoclonal antibodies (see, e.g., U.S. Pat. No. 6,084,075, incorporated herein by reference).

In another embodiment, the antibody is 1A1, 4N4, 5A13, 6D21, 6I5, 7H1, 11K2, D9, 1M11, 3N10, 5J23, 7F7, 6B11, 9B11, 9C11, 10D18 or 12F15, which are anti-CCL2 antibodies (see, e.g., WO 04/050836; and WO 03/048083, which are incorporated by reference).

In another embodiment, the antibody is AAV293, AAV294 or ABN912, which are anti-CCL2 antibodies. AAV293 is a human IgG3/κ monoclonal antibody and ABN912 is a human IgG4/κ monoclonal antibody (see, e.g., WO 02/02640 and Haringman, J. J. et al. (2006) Arthritis & Rhumatism 54(8):2387-2392, which are incorporated by reference).

In another embodiment, the antibody is C588, C750 or C751, which are anti-CCL2 antibodies (see, e.g., WO 06/085961, incorporated by reference).

### Pharmaceutical Compositions

The CCR-2 antagonists or CCL2 antagonists (e.g., peptides, polypeptides (e.g., soluble fragments of binding partners, non-naturally occurring peptides that bind CCR-2 or CCL2 (e.g., peptides obtained by phage display)), antibodies or fragments thereof, and non-protein molecules (e.g. small molecules)), can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol; and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody, unconjugated or conjugated as described herein, can include a single treatment or, preferably, can include a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

Exemplary doses for small molecule compounds include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a container (e.g., vial, or ampule), a pack, or a dispenser (e.g., a syringe) together with instructions for administration.

### Prophylactic and Therapeutic Treatment Methods

The present invention provides for both prophylactic and therapeutic methods of treating a subject at risk of (or susceptible to) a CCR-2 mediated disorder or having a CCR-2 mediated disorder, e.g., a CCR-2 mediated disorder as described herein. As used herein, the term “treatment” is defined as the application or administration of a therapeutic agent, e.g., an agent or compound which inhibits the interaction between CCR-2 and CCL2, a CCR-2 antagonist or CCL2 antagonist, to a patient, or application or administration of a therapeutic agent to an isolated tissue or cell line from a patient, who has a CCR-2 mediated disorder, a symptom of a CCR-2 mediated disorder or a predisposition toward a CCR-2 mediated disorder, with the purpose to cure, heal, alleviate, relieve, alter, remedy, ameliorate, improve or affect the disorder, the symptoms of the disorder or the predisposition toward the disorder. A therapeutic agent includes, but is not limited to, an agent or compound which inhibits the interaction between CCR-2 and CCL2, CCR-2 antagonists or CCL2 antagonists as described herein.

With regards to both prophylactic and therapeutic methods of treatment, such treatments can be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment, as described herein, based upon the patient's value for a parameter related to the patient's CCL2 expression level and/or based upon the patient's genotype at a pre-selected nucleotide or allele in the patient's CCL2 genomic sequence. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a CCR-2 mediated disorder associated with an aberrant or unwanted CCL2 expression or activity, or associated with the subject's genotype at a pre-selected nucleotide or allele in the subject's CCL2 genomic sequence, by administering to the subject a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, or by administering a treatment regimen other than a CCR-2 antagonist or CCL2 antagonist, as described herein. Subjects at risk for a CCR-2 mediated disorder which is caused or contributed to by aberrant or unwanted CCL2 expression or activity, or which is caused or contributed to by the subject's genotype at a pre-selected nucleotide or allele in the subject's CCL2 genomic sequence, can be identified by, for example, any or a combination of diagnostic or prognostic assays as described herein. Administration of a prophylactic agent can occur prior to the manifestation of symptoms characteristic of the CCR-2 mediated disorder, such that a CCR-2 mediated disorder is prevented or, alternatively, delayed in its progression.

In one embodiment, successful treatment of CCR-2 mediated disorders can be brought about by techniques that serve to inhibit the expression or activity of CCL2, e.g., the binding of CCL2 to CCR-2. In another embodiment, successful treatment of CCR-2 mediated disorders can be brought about by techniques that serve to inhibit the expression or activity of CCR-2, e.g., the binding of CCR-2 to CCL2 and/or signal transduction as a result of the binding between CCR-2 and CCL2. Successful treatment of a CCR-2 mediated disorder can be achieved using any of the agents or compounds which inhibit the interaction between CCR-2 and CCL2, CCR-2 antagonists or CCL2 antagonists described herein or any of the agents or compounds which inhibit the interaction between CCR-2 and CCL2, CCR-2 antagonists or CCL2 antagonists described in the patent applications listed in Tables III, IV and V.

The agents or compounds which inhibit the interaction between CCR-2 and CCL2, CCR-2 antagonists or CCL2 antagonists used in the present invention can be administered to a patient at therapeutically effective doses to prevent, treat or ameliorate the CCR-2 mediated disorder. A therapeutically effective dose refers to that amount of the compound sufficient to result in amelioration of a symptom of the disorder. Toxicity and therapeutic efficacy of such compounds can be determined by standard pharmaceutical procedures. For example, data obtained from cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of determination of effective dose for an individual is the ability to directly assay levels of “free” and “bound” compound in the serum of the test subject. Such assays can utilize antibody mimics and/or “biosensors” that have been created through molecular imprinting techniques. A detailed review of this technique can be seen in Ansell et al (1996) Current Opinion in Biotechnology 7:89-94 and in Shea (1994) Trends in Polymer Science 2:166-173. Such “imprinted” affinity matrixes are amenable to ligand-binding assays, whereby the immobilized monoclonal antibody component is replaced by an appropriately imprinted matrix. An example of the use of such matrixes in this way can be seen in Vlatakis et al (1993) Nature 361:645-647.

Such “imprinted” affinity matrixes can also be designed to include fluorescent groups whose photon-emitting properties measurably change upon local and selective binding of target compound. These changes can be readily assayed in real time using appropriate fiberoptic devices, in turn allowing the dose in a test subject to be quickly optimized based on its individual IC50. An rudimentary example of such a “biosensor” is discussed in Kriz et al (1995) Analytical Chemistry 67:2142-2144.

### Method of Monitoring Effectiveness of Treatment Regimens.

The methods for monitoring the effectiveness of a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof, for patients having a CCR-2 mediated disorder, include a step of determining or confirming a value for a parameter related to a patient's CCL2 expression level at a first time point, a further step of determining or confirming a value for a parameter related to a patient's CCL2 expression level at a later time point; and a further step of comparing both values to assess effectiveness of the treatment regimen.

In one embodiment, the value may be determined according to any of the detection methods described herein. In one embodiment, the first time point occurs either during or before treatment with a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. In one embodiment, the second (or later) time point occurs after the first time point, regardless of when the first time point was taken. In one embodiment, the values are compared to a reference standard. In one embodiment, comparison between the first and second value indicates a statistically significant change in CCL2 expression. In one embodiment, decreased expression of CCL2 between the first and second time points indicates that the treatment regimen is effective. In another embodiment, sustained or increased expression of CCL2 between the first and second time points indicates that the treatment regimen is ineffective.

### Method of Processing Approval of Payment or Processing of Payment of a Patient Having a CCR-2 Mediated Disorder.

The methods for processing approval of payment or processing of payment for a treatment regimen of a patient having a CCR-2 mediated disorder, e.g., a CCR-2 mediated disorder as described herein, include a step of reviewing the patient's genotype at a pre-selected nucleotide within the patient's CCL2 genomic sequence, a further step of making a decision or advising on whether payment should be made for the treatment regimen based on the patient's genotype, and a further step of transmitting or recording the decision or advice.

In one method, information, e.g., about the patient's genotype at a pre-selected nucleotide, allele or combination of alleles within the patient's CCL2 genomic sequence (e.g., the result of a genotype analysis as described herein), or about whether a patient will be responsive or non-responsive to an agent or compound which inhibits the interaction between CCR-2 and CCL2, e.g., an agent or compound which inhibits the interaction between CCR-2 and CCL2 as described herein; a CCR-2 antagonist treatment regimen, e.g. a CCR-2 antagonist treatment regimen as described herein; a CCL2 antagonist treatment regimen, e.g. a CCL2 antagonist treatment regimen as described herein; or a combination thereof, is provided (e.g., transmitted, e.g., communicated, e.g., electronically communicated) to a third party, e.g., a hospital, clinic, a government entity, a reimbursing party or insurance company (e.g., a life insurance company). For example, choice of medical procedure, payment for a medical procedure, payment by a reimbursing party, or cost for a service or insurance can be a function of the information. E.g., the third party receives the information, makes a determination based at least in part on the information, records the information, and optionally communicates the information or makes a choice of procedure, payment, level of payment, coverage, etc. based on the information.

In one embodiment, a premium for insurance (e.g., life or medical) is evaluated as a function of the genotype of an individual, e.g., the result of genotyping an individual's CCL2 genomic sequence at a pre-selected nucleotide, allele or combination of alleles as described herein, e.g., a genotype associated with responsiveness or non-responsiveness to an agent or compound which inhibits the interaction between CCR-2 and CCL2, e.g., an agent or compound which inhibits the interaction between CCR-2 and CCL2 as described herein; a CCR-2 antagonist treatment regimen, e.g. a CCR-2 antagonist treatment regimen as described herein; a CCL2 antagonist treatment regimen, e.g. a CCL2 antagonist treatment regimen as described herein; a combination thereof; or other treatment regimens, e.g., a treatment regimen described herein. For example, premiums can be increased (e.g., by a certain percentage) if an individual's genotype pre-disposes the individual to being more susceptible to CCR-2 mediated disorders. Alternatively, premiums can be decreased (e.g., by a certain percentage) if an individual's genotype pre-disposes the individual to being responsive to treatment regimens comprising, for example, an agent or compound which inhibits the interaction between CCR-2 and CCL2, e.g., an agent or compound which inhibits the interaction between CCR-2 and CCL2 as described herein; a CCR-2 antagonist, e.g. a CCR-2 antagonist as described herein; a CCL2 antagonist, e.g. a CCL2 antagonist as described herein; or a combination thereof. In another embodiment, premiums can be decreased (e.g., by a certain percentage) if an individual's genotype pre-disposes the individual to being less susceptible to CCR-2 mediated disorders. Alternatively, premiums can be increased (e.g., by a certain percentage) if an individual's genotype renders the individual to being non-responsive to treatment regimens comprising, for example, an agent or compound which inhibits the interaction between CCR-2 and CCL2, e.g., an agent or compound which inhibits the interaction between CCR-2 and CCL2 as described herein; a CCR-2 antagonist, e.g. a CCR-2 antagonist as described herein; a CCL2 antagonist, e.g. a CCL2 antagonist as described herein; or a combination thereof.

An individual's genotype information, e.g., the result of genotyping an individual's CCL2 genomic sequence at a pre-selected nucleotide, allele or combination of alleles as described herein, can be used, e.g., in an underwriting process for life insurance. The information can be incorporated into a profile about a subject. Other information in the profile can include, for example, date of birth, gender, marital status, banking information, credit information, children, and so forth. An insurance policy can be recommended as a function of the genotype information, along with one or more other items of information in the profile. An insurance premium or risk assessment can also be evaluated as a function of the genotype information. In one implementation, points are assigned on the basis of being responsive or non-responsive to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g. a CCR-2 antagonist as described herein; a CCL2 antagonist, e.g. a CCL2 antagonist as described herein; or a combination thereof.

In one embodiment, information about an individual's genotype, e.g., the result of genotyping an individual's CCL2 genomic sequence at a pre-selected nucleotide, allele or combination of alleles as described herein, is analyzed by a function that determines whether to authorize the transfer of funds to pay for a service or treatment provided to a subject (or make another decision referred to herein). For example, the genotyping results may indicate that a subject is responsive or non-responsive to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g. a CCR-2 antagonist as described herein; a CCL2 antagonist, e.g. a CCL2 antagonist as described herein; or a combination thereof, suggesting that a treatment course is needed, thereby triggering an outcome that indicates or causes authorization to pay for a service or treatment provided to a subject. In one example, a subject's genotype is determined and payment is authorized if the informative expression level identifies a responsive patient. For example, an entity, e.g., a hospital, care giver, government entity, or an insurance company or other entity which pays for, or reimburses medical expenses, can use the outcome of a method described herein to determine whether a party, e.g., a party other than the subject patient, will pay for services (e.g., a particular therapy) or treatment provided to the patient. For example, a first entity, e.g., an insurance company, can use the outcome of a method described herein to determine whether to provide financial payment to, or on behalf of, a patient, e.g., whether to reimburse a third party, e.g., a vendor of goods or services, a hospital, physician, or other care-giver, for a service or treatment provided to a patient. For example, a first entity, e.g., an insurance company, can use the outcome of a method described herein to determine whether to continue, discontinue, enroll an individual in an insurance plan or program, e.g., a health insurance or life insurance plan or program.

In one aspect, the disclosure features a method of providing, e.g., transmitting data. The method includes providing data described herein, e.g., generated by a method described herein, to provide a record, e.g., a record described herein, for determining if a payment will be provided. In some embodiments, the data is provided by computer, compact disc, telephone, facsimile, email, or letter. In some embodiments, the data is provided by a first party to a second party. In some embodiments, the first party is selected from the subject, a healthcare provider, a treating physician, a health maintenance organization (HMO), a hospital, a governmental entity, or an entity which sells or supplies the drug. In some embodiments, the second party is a third party payor, an insurance company, employer, employer sponsored health plan, HMO, or governmental entity. In some embodiments, the first party is selected from the subject, a healthcare provider, a treating physician, an HMO, a hospital, an insurance company, or an entity which sells or supplies the drug and the second party is a governmental entity. In some embodiments, the first party is selected from the subject, a healthcare provider, a treating physician, an HMO, a hospital, an insurance company, or an entity which sells or supplies the drug and the second party is an insurance company.

In another aspect, the disclosure features a record (e.g., computer readable record) which includes the subject's genotype.

### Arrays and Kits for Evaluating a Patient Having a CCR-2 Mediated Disorder

In another aspect, the invention features an array including a substrate having a plurality of addresses for evaluating a patient having a CCR-2 mediated disorder, e.g., a CCR-2 mediated disorder described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof. In another aspect, the invention features a kit for evaluating a patient having a CCR-2 mediated disorder, e.g., a CCR-2 mediated disorder described herein, for responsiveness or non-responsiveness to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein; or a combination thereof.

### Electronic Apparatus Readable Arrays

Electronic apparatus, including readable arrays comprising at least one marker, e.g., CCL2, or pre-selected nucleotide, allele or combination of alleles, e.g., a pre-selected nucleotide, allele or combination of alleles with a subject's CCL2 genomic sequence, e.g., nucleotide 2485 as numbered in SEQ ID NO:1, or a nucleotide or allele in linkage disequilibrium with the nucleotide at position 2485, e.g., nucleotides 2236, 2936 or 5837 as numbered in SEQ ID NO:1, of the present invention is also contemplated for use in conjunction with the methods of the invention. As used herein, “electronic apparatus readable media” refers to any suitable medium for storing, holding or containing data or information that can be read and accessed directly by an electronic apparatus. As used herein, the term “electronic apparatus” is intended to include any suitable computing or processing apparatus or other device configured or adapted for storing data or information. Examples of electronic apparatus suitable for use with the present invention and monitoring of the recorded information include stand-alone computing apparatus; networks, including a local area network (LAN), a wide area network (WAN) Internet, Intranet, and Extranet; electronic appliances such as a personal digital assistants (PDAs), cellular phone, pager and the like; and local and distributed processing systems. As used herein, “recorded” refers to a process for storing or encoding information on the electronic apparatus readable medium. Those skilled in the art can readily adopt any of the presently known methods for recording information on known media to generate manufactures comprising the markers of the present invention.

In one embodiment, the array comprises a plurality of addresses. Each address of the plurality includes a unique (as compared to a nucleic acid at another address) nucleic acid (e.g., a DNA or an RNA) which binds a target, wherein at least one of the nucleic acids binds to a CCL2 region and can distinguish between a first CCL2 allele and a second CCL2 allele, wherein the first allele expresses CCL2 at a higher rate than the second. In one embodiment, at least one of the nucleic acids can distinguish between a single nucleotide difference in two CCL2 sequences. For example, in one embodiment, at least one of the nucleotides can distinguish between two CCL2 sequences which differ at: a pre-selected nucleotide or allele within the patient's CCL2 genomic sequence; a pre-selected nucleotide or allele within a non-coding region of the CCL2 genomic sequence (e.g., in an intron or a regulatory region of the CCL2 genomic sequence); at position 2485 as numbered in SEQ ID NO:1, or one or more nucleotides in linkage disequilibrium with the nucleotide at position 2485 as numbered in SEQ ID NO:1, e.g., nucleotides selected from the group consisting of a nucleotide at position 2236 as numbered in SEQ ID NO:1, a nucleotide at position 2936 as numbered in SEQ ID NO:1, and a nucleotide at position 5837 as numbered in SEQ ID NO:1.

In one embodiment, at least one of the nucleotides can distinguish a G and an A at the nucleotide at position 2485 as numbered in SEQ ID NO:1.

In one embodiment, at least one of the nucleotides binds specifically to a CCL2 genomic sequence having a G at the nucleotide at position 2485 as numbered in SEQ ID NO:1.

In one embodiment, at least one of the nucleotides binds specifically to a CCL2 genomic sequence having an A at the nucleotide at position 2485 as numbered in SEQ ID NO:1.

In one embodiment, at least one of the nucleotides binds specifically to a CCL2 genomic sequence having an A at the nucleotide at position 2485 as numbered in SEQ ID NO:1 and at least one nucleotides binds specifically to a CCL2 genomic sequence having a G at the nucleotide at position 2485 as numbered in SEQ ID NO:1.

In one embodiment, at least one of the nucleotides can distinguish a C and an A at the nucleotide at position 2236 as numbered in SEQ ID NO:1.

In one embodiment, at least one of the nucleotides binds specifically to a CCL2 genomic sequence having a C at the nucleotide at position 2236 as numbered in SEQ ID NO:1.

In one embodiment, at least one of the nucleotides binds specifically to a CCL2 genomic sequence having an A at the nucleotide at position 2236 as numbered in SEQ ID NO:1.

In one embodiment, at least one of the nucleotides binds specifically to a CCL2 genomic sequence having a C at the nucleotide at position 2236 as numbered in SEQ ID NO:1 and at least one nucleotides binds specifically to a CCL2 genomic sequence having an A at the nucleotide at position 2236 as numbered in SEQ ID NO:1.

In one embodiment, at least one of the nucleotides can distinguish a T and an A at the nucleotide at position 2936 as numbered in SEQ ID NO:1.

In one embodiment, at least one of the nucleotides binds specifically to a CCL2 genomic sequence having a T at the nucleotide at position 2936 as numbered in SEQ ID NO:1.

In one embodiment, at least one of the nucleotides binds specifically to a CCL2 genomic sequence having an A at the nucleotide at position 2936 as numbered in SEQ ID NO:1.

In one embodiment, at least one of the nucleotides binds specifically to a CCL2 genomic sequence having a T at the nucleotide at position 2936 as numbered in SEQ ID NO:1 and at least one nucleotides binds specifically to a CCL2 genomic sequence having an A at the nucleotide at position 2936 as numbered in SEQ ID NO:1.

In one embodiment, at least one of the nucleotides can distinguish a C and a G at the nucleotide at position 5837 as numbered in SEQ ID NO:1.

In one embodiment, at least one of the nucleotides binds specifically to a CCL2 genomic sequence having a C at the nucleotide at position 5837 as numbered in SEQ ID NO:1.

In one embodiment, at least one of the nucleotides binds specifically to a CCL2 genomic sequence having a G at the nucleotide at position 5837 as numbered in SEQ ID NO:1.

In one embodiment, at least one of the nucleotides binds specifically to a CCL2 genomic sequence having a C at the nucleotide at position 5837 as numbered in SEQ ID NO:1 and at least one nucleotides binds specifically to a CCL2 genomic sequence having a G at the nucleotide at position 5837 as numbered in SEQ ID NO:1.

In one embodiment, at least one of the nucleotides can distinguish haplotype H1, as defined in Table II above, from other haplotypes present in the CCL2 genomic sequence.

In one embodiment, the array has less than 1000, 500, 100, 50 or 20 unique nucleic acids or addresses. In one embodiment, the array comprises nucleic acids encoding less than 1000, 500, 100, 50 or 20 unique peptides.

In one embodiment, the array is used for evaluating responsiveness or non-responsiveness to a treatment regimen of a patient having a CCR-2 mediated disorder, e.g. a CCR-2 mediated disorder described herein, to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g. a CCR-2 antagonist as described herein; a CCL2 antagonist, e.g. a CCL2 antagonist as described herein; or a combination thereof

In another embodiment, the array is used to determining whether the patient has a CCR-2 mediated disorder, e.g. a CCR-2 mediated disorder described herein, based upon the presence or absence of a pre-selected nucleotide at position 2485 as numbered in SEQ ID NO:1, or one or more nucleotides in linkage disequilibrium with the nucleotide at position 2485 as numbered in SEQ ID NO:1. In other embodiments, the array is used to determine whether a patient having or at risk for a CCR-2 mediated disorder is predisposed to respond or not respond to a treatment regimen, e.g., a treatment regimen described herein.

In one embodiment, the CCR-2 mediated disorder is a CCR-2 mediated disorder described herein, e.g., a cardiovascular disorder or atherosclerosis.

In one embodiment, the array includes a sample obtained from a patient having a CCR-2 mediated disorder, e.g., atherosclerosis.

Microarray systems are well known and used in the art for assessment of samples, whether by assessing, for example, gene expression (e.g., RNA detection, protein detection), genotype, or metabolite production. Microarrays for use according to the invention include one or more probes of marker(s), e.g., CCL2, or one or more nucleic acid sequences as discussed above which are characteristic of response and/or non-response to a treatment regimen, e.g., a treatment regimen as described herein. In one embodiment, the microarray comprises one or more nucleic acids, as described above, capable of determining a subject's genotype in order to assess the subject for responsiveness or non-responsiveness to a treatment regimen, e.g., a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g. a CCR-2 antagonist as described herein; a CCL2 antagonist, e.g. a CCL2 antagonist as described herein; or a combination thereof. In another embodiment, the microarray comprises one or more probes corresponding to one or more markers, e.g., CCL2, which demonstrates decreased expression in patients responsive to a treatment regimen, e.g., a treatment regimen as described herein. A number of different microarray configurations and methods for their production are known to those of skill in the art and are disclosed, for example, in U.S. Pat. Nos. 5,242,974; 5,384,261; 5,405,783; 5,412,087; 5,424,186; 5,429,807; 5,436,327; 5,445,934; 5,556,752; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,624,711; 5,700,637; 5,744,305; 5,770,456; 5,770,722; 5,837,832; 5,856,101; 5,874,219; 5,885,837; 5,919,523; 5981185; 6,022,963; 6,077,674; 6,156,501; 6,261,776; 6,346,413; 6,440,677; 6,451,536; 6,576,424; 6,610,482; 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and 5,874,219; Shena, et al. (1998) Tibtech 16:301, 1998; Duggan, et al. (1999) Nat. Genet. 21:10; Bowtell, et al. (1999) Nat. Genet. 21:25; Lipshutz, et al. (1999) 21 Nature Genet. 20-24, 1999; Blanchard, et al. (1996) Biosensors and Bioelectronics 11:687-90, 1996; Maskos, et al. (1993) Nucleic Acids Res. 21:4663-69, 1993; Hughes, et al. (2001) Nat. Biotechol. 19:342; each of which is herein incorporated by reference. A tissue microarray can be used for protein identification (see Hans et al. (2004) Blood 103:275-282). A phage-epitope microarray can be used to identify one or more proteins in a sample based on whether the protein or proteins induce auto-antibodies in the patient (Bradford et al. (2006) Urol. Oncol. 24:237-242).

A microarray thus comprises one or more probes corresponding to one or more pre-selected nucleotides or alleles within the CCL2 genomic sequence, or markers, e.g., CCL2, of the present invention. The microarray may comprise probes corresponding to, for example, at least 10, at least 20, at least 50, at least 100, or at least 1000 pre-selected nucleotides or alleles within the CCL2 genomic sequence, as described herein, or markers, e.g., CCL2, of the invention which are characteristic of patient response to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g. a CCR-2 antagonist as described herein; a CCL2 antagonist, e.g. a CCL2 antagonist as described herein; or a combination thereof. The microarray can be used to assay presence of one or more alleles, or to assay expression levels of markers, e.g., CCL2. In one example, the array can be used to assay more than one pre-selected nucleotide or allele within a subject's CCL2 genomic sequence in a sample to ascertain the subject's genotype. In another example, the array can be used to assay the expression level of one or more than one marker, e.g., CCL2, in a sample to ascertain an expression profile of the marker(s) in the array. In this manner, up to about 44,000 pre-selected nucleotides or alleles, or markers can be simultaneously assayed. This allows a genotype or profile to be developed showing a battery of pre-selected nucleotides or markers specifically expressed in one or more samples. Still further, this allows a profile to be developed to assess responsiveness to one or more treatment regimens, e.g., a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g. a CCR-2 antagonist as described herein; a CCL2 antagonist, e.g. a CCL2 antagonist as described herein; or a combination thereof.

The array is also useful for ascertaining differential expression patterns of one or more markers in cells or samples obtained from normal individuals and individuals having a CCR-2 mediated disorder. This provides a battery of predictive markers that could serve as a tool for ease of identification of responsive and non-responsive patients. Further, the array is useful for ascertaining expression of reference markers for reference expression levels. In another example, the array can be used to monitor the time course of expression of one or more predictive markers in the array.

### Reagents and Kits

The invention also encompasses kits comprising reagents for detecting the presence of a polypeptide or for detecting a nucleic acid corresponding to a marker in a sample (e.g. a biological sample taken from a patient having a CCR-2 mediated disorder). The invention also encompasses kits for detecting alleles within a genomic sequence in a sample (e.g. a biological sample taken from a patient having a CCR-2 mediated disorder). Such kits can be used to determine if a subject is predisposed to response or non-response to a treatment regimen comprising an agent or compound which inhibits the interaction between CCR-2 and CCL2, e.g., an agent or compound which inhibits the interaction between CCR-2 and CCL2 as described herein; a CCR-2 antagonist, e.g. a CCR-2 antagonist as described herein; a CCL2 antagonist, e.g. a CCL2 antagonist as described herein; a combination thereof; or other treatment regimen, e.g., other treatment regimen described herein. In another aspect, the invention provides a test kit for monitoring the efficacy of a compound or therapeutic in a sample. For example, the kit may comprise reagents, e.g., a labeled probe capable of detecting a polypeptide or an mRNA encoding a polypeptide corresponding to a marker of the invention, or a probe capable of detecting an allele within a subject's genomic sequence, e.g., within the genomic sequence of the subject's CCL2 gene, in a biological sample and means for determining the amount of the polypeptide or mRNA in the sample (e.g., an antibody which binds the polypeptide or an oligonucleotide probe which binds to DNA or mRNA encoding the polypeptide), or means for determining the subject's genotype. Kits may further include instructions for use of the provided kits and interpreting the results obtained using the kit; additional reagents for preparation of probes for use in the methods provided; and detectable label, alone or conjugated to the provided probe(s).

For antibody-based kits, the kit can comprise reagents, for example: (1) a first antibody (e.g., attached to a solid support) which binds to a polypeptide corresponding to a marker of the invention, e.g., CCL2; and, optionally, (2) a second, different antibody which binds to either the polypeptide or the first antibody and is conjugated to a detectable label.

For oligonucleotide-based kits, the kit can comprise reagents, for example: (1) an oligonucleotide, e.g., a detectably labeled oligonucleotide, which hybridizes to a nucleic acid sequence within a subject's genomic sequence, e.g., within a subject's CCL2 genomic sequence; (2) an oligonucleotide, e.g., a detectbaly labeled oligonucleotide, which hybridizes to a nucleic acid sequence encoding a polypeptide corresponding to a marker of the invention, e.g., CCL2; (3) a pair of primers useful for amplifying a nucleic acid molecule corresponding to a subject's genomic sequence; (4) a pair of primers useful for amplifying a nucleic acid encoding a marker of the invention; (5) a primer set comprising oligonucleotides which hybridize to at least two nucleic acid sequences within a subject's genomic sequence, e.g., within a subject's CCL2 genomic sequence; or (6) a primer set comprising oligonucleotides which hybridize to at least two nucleic acid sequences encoding polypeptide predictive markers of the invention, e.g., CCL2.

In one embodiment, the kit comprises at least one nucleic acid sequence capable of distinguishing a G and an A at the nucleotide at position 2485 as numbered in SEQ ID NO:1.

In one embodiment, the kit comprises at least one nucleic acid sequence which binds specifically to a CCL2 genomic sequence having a G at the nucleotide at position 2485 as numbered in SEQ ID NO:1.

In one embodiment, the kit comprises at least one nucleic acid sequence which binds specifically to a CCL2 genomic sequence having an A at the nucleotide at position 2485 as numbered in SEQ ID NO:1.

In one embodiment, the kit comprises at least one nucleic acid sequence which binds specifically to a CCL2 genomic sequence having an A at the nucleotide at position 2485 as numbered in SEQ ID NO:1 and at least one nucleic acid sequence which binds specifically to a CCL2 genomic sequence having a G at the nucleotide at position 2485 as numbered in SEQ ID NO:1.

In one embodiment, the kit comprises at least one nucleic acid sequence capable of distinguishing a C and an A at the nucleotide at position 2236 as numbered in SEQ ID NO:1.

In one embodiment, the kit comprises at least one nucleic acid sequence which binds specifically to a CCL2 genomic sequence having a C at the nucleotide at position 2236 as numbered in SEQ ID NO:1.

In one embodiment, the kit comprises at least one nucleic acid sequence which binds specifically to a CCL2 genomic sequence having an A at the nucleotide at position 2236 as numbered in SEQ ID NO:1.

In one embodiment, the kit comprises at least one nucleic acid sequence which binds specifically to a CCL2 genomic sequence having a C at the nucleotide at position 2236 as numbered in SEQ ID NO:1 and at least one nucleic acid sequence which binds specifically to a CCL2 genomic sequence having an A at the nucleotide at position 2236 as numbered in SEQ ID NO:1.

In one embodiment, the kit comprises at least one nucleic acid sequence capable of distinguishing a T and an A at the nucleotide at position 2936 as numbered in SEQ ID NO:1.

In one embodiment, the kit comprises at least one nucleic acid sequence which binds specifically to a CCL2 genomic sequence having a T at the nucleotide at position 2936 as numbered in SEQ ID NO:1.

In one embodiment, the kit comprises at least one nucleic acid sequence which binds specifically to a CCL2 genomic sequence having an A at the nucleotide at position 2936 as numbered in SEQ ID NO:1.

In one embodiment, the kit comprises at least one nucleic acid sequence which binds specifically to a CCL2 genomic sequence having a T at the nucleotide at position 2936 as numbered in SEQ ID NO:1 and at least one nucleic acid sequence which binds specifically to a CCL2 genomic sequence having an A at the nucleotide at position 2936 as numbered in SEQ ID NO:1.

In one embodiment, the kit comprises at least one nucleic acid sequence capable of distinguishing a C and a G at the nucleotide at position 5837 as numbered in SEQ ID NO:1.

In one embodiment, the kit comprises at least one nucleic acid sequence which binds specifically to a CCL2 genomic sequence having a C at the nucleotide at position 5837 as numbered in SEQ ID NO:1.

In one embodiment, the kit comprises at least one nucleic acid sequence which binds specifically to a CCL2 genomic sequence having a G at the nucleotide at position 5837 as numbered in SEQ ID NO:1.

In one embodiment, the kit comprises at least one nucleic acid sequence which binds specifically to a CCL2 genomic sequence having a C at the nucleotide at position 5837 as numbered in SEQ ID NO:1 and at least one nucleic acid sequence which binds specifically to a CCL2 genomic sequence having a G at the nucleotide at position 5837 as numbered in SEQ ID NO:1.

In one embodiment, the kit comprises at least one nucleic acid sequence which is capable of distinguishing haplotype H1, as defined in Table II above, from other haplotypes present in the CCL2 genomic sequence.

In one embodiment, the kit comprises one or more nucleic acid sequence described above.

The kit can also comprise, e.g., a buffering agent, a preservative, or a protein stabilizing agent. The kit can further comprise components necessary for detecting the detectable label (e.g., an enzyme or a substrate). For nucleic acid sets (as described above), the kit can comprise a set of nucleic acid sequences on an array or chip for use in determining a subject's genotype. For marker sets, the kit can comprise a marker set array or chip for use in detecting the predictive markers. The kit can also contain a reference sample or a series of reference samples which can be assayed and compared to the test sample. Each component of the kit can be enclosed within an individual container and all of the various containers can be within a single package, along with instructions for interpreting the results of the assays performed using the kit.

### Articles of Manufacture

The invention also encompasses articles of manufacture comprising i) a pharmaceutical composition, as described herein, comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein, a CCL2 antagonist, e.g., a CCL2 antagonist as described herein, or combination thereof; and ii) instructions for determining the appropriateness of use of the pharmaceutical composition by determining a patient's genotype at a pre-selected nucleotide within the patient's CCL2 genomic sequence, or by determining the patient's haplotype of the patient's CCL2 genomic sequence, according to the methods described above. In one embodiment, the pre-selected nucleotide is the nucleotide at position 2485, as numbered in SEQ ID NO:1, or a nucleotide, allele or combination of alleles in linkage disequilibrium with the nucleotide at position 2485 as numbered in SEQ ID NO:1. In one embodiment, the nucleotide or allele in linkage disequilibrium with the nucleotide at position 2485, as numbered in SEQ ID NO:1, is nucleotide at position 2236, as numbered in SEQ ID NO:1, nucleotide at position 2936, as numbered in SEQ ID NO:1, or nucleotide at position 5837, as numbered in SEQ ID NO:1. In one embodiment, the patient is selected for treatment and/or is treated with the treatment regimen determined to be appropriate based upon the determination of the patient's genotype or haplotype, as described herein.

The invention also encompasses articles of manufacture comprising i) reagents for making a determination of a patient's genotype at a pre-selected nucleotide within a subject's CCL2 genomic sequence and/or reagents for making a determination of a patient's haplotype of the patient's CCL2 genomic sequence, e.g., reagents as described herein; and ii) instructions for determining the appropriateness of use of a pharmaceutical composition comprising an agent or compound which inhibits the interaction between CCR-2 and CCL2; e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein, or a combination thereof, on the basis of said determination. In one embodiment, the pre-selected nucleotide is the nucleotide at position 2485, as numbered in SEQ ID NO:1, or a nucleotide, allele or combination of alleles in linkage disequilibrium with the nucleotide at position 2485 as numbered in SEQ ID NO:1. In one embodiment, the nucleotide or allele in linkage disequilibrium with the nucleotide at position 2485, as numbered in SEQ ID NO:1, is nucleotide at position 2236, as numbered in SEQ ID NO:1, nucleotide at position 2936, as numbered in SEQ ID NO:1, or nucleotide at position 5837, as numbered in SEQ ID NO:1.

The invention also encompasses articles of manufacture comprising i) a pharmaceutical composition, as described herein, comprising an agent or compound which inhibits the interaction between CCR-2 and CCL2, e.g., an agent or compound which inhibits the interaction between CCR-2 and CCL2 as described herein; a CCR-2 antagonist, e.g., a CCR-2 antagonist as described herein, or a CCL2 antagonist, e.g., a CCL2 antagonist as described herein; ii) reagents for making a determination of a patient's genotype at a pre-selected nucleotide within a subject's CCL2 genomic sequence and/or reagents for making a determination of a patient's haplotype of the patient's CCL2 genomic sequence, e.g., reagents as described herein; and iii) instructions for determining the appropriateness of use of a pharmaceutical composition comprising an agent or compound which inhibits the interaction between CCR-2 and CCL2; e.g., an agent or compound which inhibits the interaction between CCR-2 and CCL2 as described herein; a CCR-2 antagonist, e.g., a CCR-2 antagonist as described herein; a CCL2 antagonist, e.g., a CCL2 antagonist as described herein, or a combination thereof, on the basis of said determination. In one embodiment, the nucleotide or allele in linkage disequilibrium with the nucleotide at position 2485, as numbered in SEQ ID NO:1, is nucleotide at position 2236, as numbered in SEQ ID NO:1, nucleotide at position 2936, as numbered in SEQ ID NO:1, or nucleotide at position 5837, as numbered in SEQ ID NO:1. In one embodiment, the patient is selected for treatment and/or is treated with the treatment regimen determined to be appropriate based upon the determination of the patient's genotype or haplotype, as described herein.

This invention is further illustrated by the following exemplification which should not be construed as limiting. The contents of all references, patents and published patent applications, e.g., patents and patent applications cited in Tables III, IV and V, cited throughout this application are hereby incorporated by reference.

### Exemplification

Study Objectives: To determine the effect of MLN1202 on levels of CRP or other inflammatory biomarkers of cardiovascular disease and to investigate the correlation of genetic single nucleotide polymorphisms in genes in the CCR2/CCL2 pathway to CRP reduction in patients receiving a single dose of MLN1202.

Study Design, Conduct, and Patients: A double-blinded, randomized, placebo-controlled trial of 110 patients (2 cohorts, 55 patients each) measuring the effects of MLN1202 on CRP levels in patients with 2 or more risk factors for cardiovascular disease (CVD) and elevated CRP (>3 mg/L) at nine centers within the United States. Patients were randomized to receive MLN1202, 10 mg/kg, as a single dose intravenous (IV) infusion or matching placebo, as a single dose IV infusion. The screening period was 2 weeks and study conduct (from Screening Days −14 to −1 to end of study visit Day 113) was 4 months. Participants were permitted to take other medications and adhere to the treatment plan prescribed by their treating physician prior to enrollment. There was no follow-up period for this study.

### Inclusion Criteria

Male or female (if female, non-childbearing potential by surgical sterilization or postmenopausal) ages 35 to 75 years;

Ability to comply with the protocol for the duration of the study;

Ability to use adequate “double-barrier” contraceptive methods for the duration of the study;

Presence of two or more risk factors for CVD selected from the group consisting of:
i) History of or concurrent cigarette smoking ≧20 pack years;
ii) Hypertension with concurrent control to a blood pressure of <140/90 on anti-hypertensive medications;
iii) Body mass index (BMI) defined as weight (kg) divided by height (in m²) ≧30;
iv) Hypercholesterolemia defined as fasting LDL >130 mg/dL;
v) High-density lipoprotein (HDL) levels of 40 mg/dl or less;
vi) Type II diabetes mellitus; and
vii) Family history of a definable CVD event (myocardial infarction [MI] or intervention) in a first-degree relative prior to the age of 55 (male) or 65 (female).

Patients with hypercholesterolemia may be on stable doses of lipid lowering agents with no change in regimen/dose in <60 days;

Patients having hsCRP (peripheral blood plasma) levels >3.0 mg/L on more than 1 test date in the last 12 months, separated by at least 2 weeks, with the most recent CRP measured within 14 days of randomization.

### Exclusion Criteria

History of stroke;

Diagnosis of congestive heart failure (CHF) or clinical evidence suggesting CHF including history of orthopnea, exertional dyspnea, pedal edema, pulmonary rales, hepatosplenomegally, cardiomegally, and a prodiastolic (S3) gallop;

Concurrent chronic inflammatory illness (inflammatory bowel disease (IBD), rheumatoid arthritis (RA), chronic obstructive pulmonary disease (COPD), systemic lupus erythematosus (SLE), etc.) or concurrent chronic infectious illness (human immunodeficiency virus (HIV), tuberculosis (TB), osteomyelitis);

Concurrent use of anti-inflammatory or disease modifying agents (methotrexate, azathioprine, corticosteroids, others). Aspirin, clopidigrel, and nonsteroidal anti-inflammatory drugs (NSAIDs) are acceptable;

History of cancer with the exception of cervical carcinoma-in-situ or basal cell carcinoma of the skin;

History of receiving live attenuated vaccine within last 60 days;

Enrollment in a clinical trial of an investigational agent in the last 60 days;

History of illicit drug use or alcohol abuse in the last five years;

Concurrent use of anti-inflammatory, disease modifying agents (methotrexate, azathioprine, oral or systemic corticosteroids, others.). Inhaled corticosteroids, aspirin, clopidigrel, and NSAIDs are acceptable;

History or planned percutaneous coronary intervention (PCI) or coronary artery bypass graft (CABG) or other vascular surgery planned within study duration;

A chest X-ray within 6 months prior to study entry with clinically significant findings or abnormalities, in the opinion of the investigator;

A screening ECG within 2 weeks prior to study entry with clinically significant abnormalities or findings, in the opinion of the investigator;

A known inadequate renal function (serum creatinine ≧1.5 times the upper limit of normal);

A known inadequate hepatic function (aspartate aminotransferase [AST], alanine aminotransferase [ALT] or total bilirubin ≧2 times the upper limit of normal);

Pregnant or breast-feeding or planning pregnancy; and

Any other reason in the opinion of the investigator that a subject should not be enrolled in this study.

Main Outcome Measure: Change from baseline to Day 57 in CRP between subjects with baseline elevation of CRP (>3 mg/L) treated with placebo compared with MLN1202.

Study Methods: Patients with risk factors for CVD were screened for inclusion and exclusion criteria including assessment of CRP. If the patient's CRP was greater than 3.0 mg/L on repeated measurements at least 2 weeks apart, the patient was enrolled and randomized to receive placebo or one dose of MLN1202 delivered intravenously at start of study. A medical history was completed, including detailed information on comorbidities, concomitant medications, and cardiovascular history, including current status. Treating physicians examined the patients at each study visit and completed case report forms. Patients were tested for CRP levels prior to dosing on Day 1, and on Days 1, 15, 29, 43, 57, 71, 85, and 113. Peripheral blood was tested for levels of MLN1202 (pharmacokinetic marker); receptor saturation on CD14⁺ cells (pharmacodynamic marker); the presence of human anti-human antibodies (HAHA); hemoglobin A1c; lipid profile; and a limited panel of chemokines and cytokines. DNA was isolated for genotyping. Safety was assessed by physical examination, clinical laboratories, and adverse event (AE) reporting from Day 1 to Day 113 of the trial.

All blood samples were collected and processed immediately after sampling. The genotypes of pre-selected nucleotides within the CCR-2 (rs1799864) or CCL2 (rs1024611) genomic sequences of each subject enrolled in the study were determined using a pre-validated Taqman assay (Applied Biosystems Inc., Foster City, Calif.) and an Applied Biosystems Inc 7900HT instrument.

Data Analysis: All data were analyzed according to a pre-established statistical analysis plan and according to the intention-to treat principle. The Intent to Treat (ITT) population includes all patients who are randomized, regardless of whether or not they received treatment. The efficacy parameter, CRP, was collected at Days 1, 3 (in the first 20 patients only), 8, 15, 29, 43, 57, 71, 85 and 113 for all patients. If a patient withdrew from the study or missed a visit after Day 29, the last previous non-missing observation (at Day 29 or later) was carried forward (Last Observed Carried Forward (LOCF) for subsequent assessments. The primary efficacy endpoint of this study is the change from baseline to Day 57 in CRP levels. The change from baseline CRP level for the MLN1202 group was compared with the change from baseline CRP levels for the placebo group at Day 57 using a Wilcoxon rank sum test. A secondary analysis for the primary efficacy endpoint was generated to compare the change from baseline to Day 57 in CRP levels between the placebo and the MLN1202 treatment group adjusting for ranked baseline CRP. An analysis of covariance (ANCOVA) or ranked changes was performed while controlling for the ranked baseline CRP levels. For the secondary endpoint of time to 1.0 mg/L drop in CRP, Kaplan-Meier estimates were used and the log-rank test was performed for the comparison of treatment groups.

Potential biomarkers were analyzed by first determining the association between each potential biomarker and CRP using Pearson correlations. The change in each marker from baseline over time was assessed by repeat-measures analysis of variance (ANOVA) using treatment as a factor (placebo or MLN1202) to test the hypothesis that treatment is significantly associated with change in some or all of the markers. Finally, each marker was assessed on whether change by Day 15 or Day 29 predicts change in CRP at subsequent time points. Markers that show such responses were considered for further evaluation in subsequent studies to validate their ability to predict change in CRP and clinical outcomes in response to MLN1202. CCL2 was measured in serum at baseline and at the time of CRP measurements to evaluate the biological activity of MLN1202 on CCR-2/CCL2 pathway.

The potential association of SNPs in the genes encoding CCL2 and CCR-2 with clinical outcome was tested using linear regression. Briefly, a generalized linear model was developed in which the dependent variable, serum CRP levels following treatment, was controlled for baseline serum CRP, and 2 independent variables were tested for association along with their interaction term. The independent variables are dose cohort (active treatment versus placebo) and genotype at the SNP under investigation.

### Results:

### Baseline Characteristics

A total of 112 patients were enrolled, randomized and 106 completed all eleven visits. All 108 participants were analyzed as randomized in the intention-to-treat analysis using the last observation carried forward approach. There were no significant differences in the baseline characteristics of study groups between genotypes or in baseline values of the biomarker data.

### Primary Endpoint

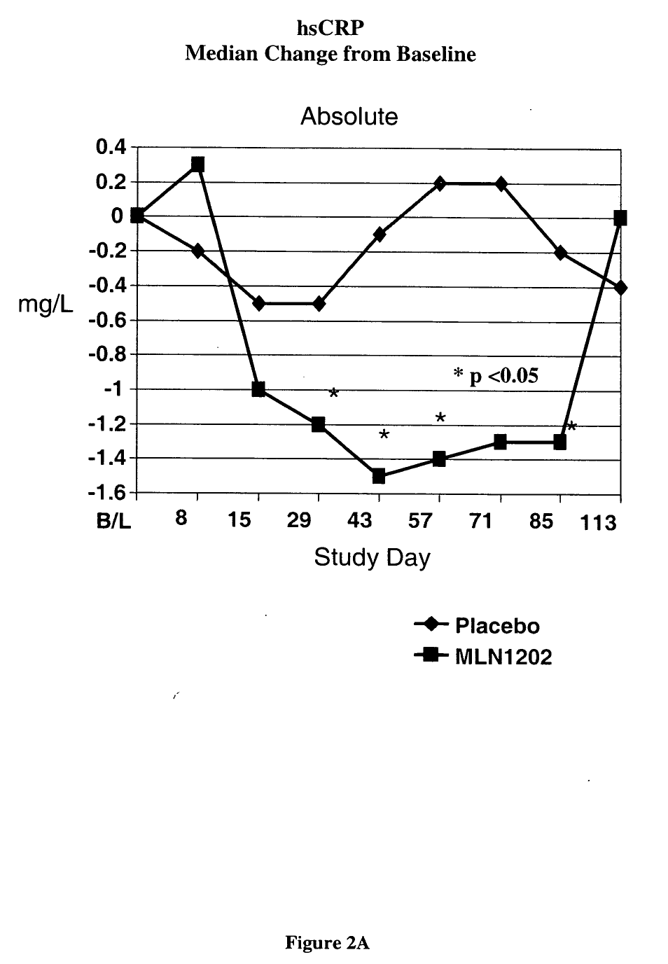

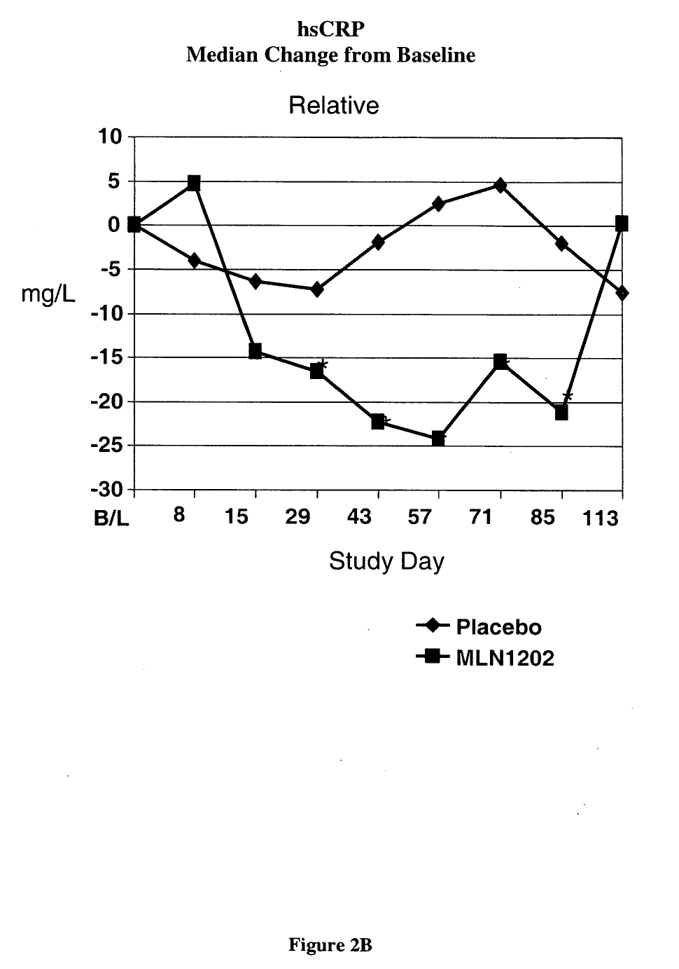

In response to 10 mg/kg MLN1202, levels of CRP were significantly reduced by 25% (P=0.007) at Day 57. MLN1202 was well tolerated and was not associated with any serious adverse events or significant HAHA. (FIGS. 2A-B)

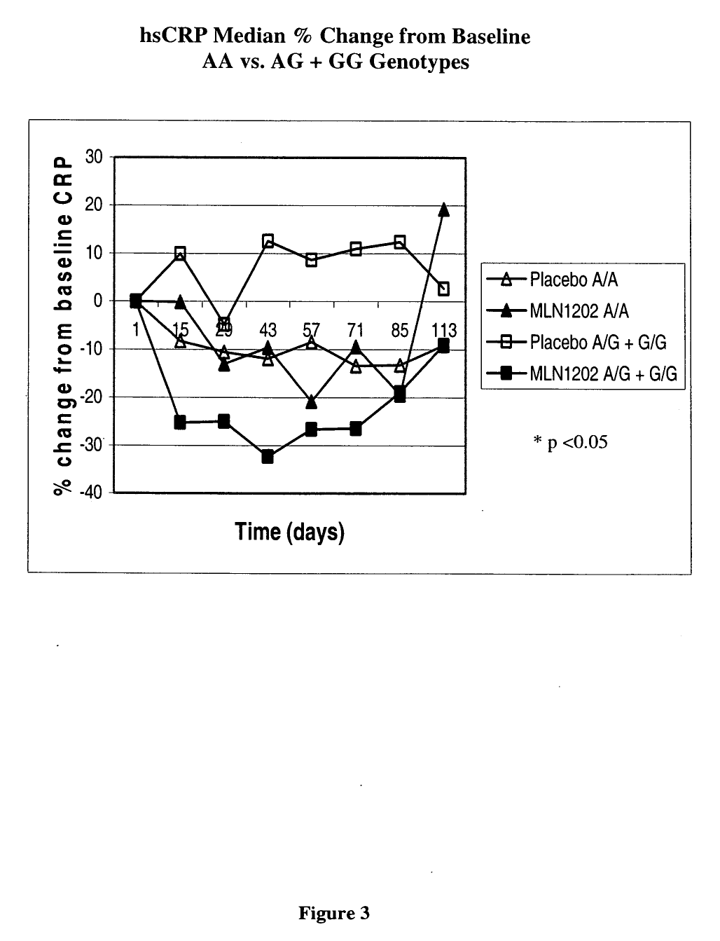

However, when these data were examined in combination with each of the patient's genotype at the pre-selected nucleotide within the patient's CCL2 genomic sequence at nucleotide position 2485, as numbered in SEQ ID NO:1 (rs1024611), it became clear that the effect on CRP levels was almost completely limited to those subjects who carry one or two A→G mutations at the polymorphic site at nucleotide position 2485, as numbered in SEQ ID NO:1 (rs1024611) (FIG. 3). In other words, patients homozygous or heterozygous for the G allele at nucleotide position 2485, as numbered in SEQ ID NO:1 (rs1024611), who were treated with MLN1202 responded well to the treatment regimen as their CRP levels were reduced as compared to patients homozygous for the A allele. The frequencies of the patients' genotypes at the polymorphic site at nucleotide position 2485, as numbered in SEQ ID NO:1 (rs1024611), in this study were:

| | |
|---|---|
| G/G - 14 | (13%) |
| G/A - 43 | (40%) |
| A/A - 50 | (47%) |

Therefore, 53% of the study population were homozygous or heterozygous for the G allele at nucleotide position 2485, as numbered in SEQ ID NO:1 (rs1024611). Hence, 53% of the study population carried the susceptibility allele for responding to MLN1202.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a patient having a CC-chemokine receptor 2 (CCR-2) mediated disorder, comprising:
determining a patient's genotype of a nucleotide at position 2485 as numbered in SEQ ID NO:1, and
administering to the patient a treatment regimen based upon the patient's genotype, wherein i) if the patient has a G allele at the nucleotide at position 2485 as numbered in SEQ ID NO:1, then selecting or administering a first treatment regimen, and ii) if the patient is homozygous for the A allele at the nucleotide at position 2485 as numbered in SEQ ID NO:1, then selecting or administering a second treatment regimen which is different from the first treatment regimen, to thereby treat the CCR-2 mediated disorder.

2. The method of claim 1, wherein the presence or absence of the G allele at the nucleotide at position 2485 is determined by i) determining if the G allele is present at position 2485 as numbered in SEQ ID NO:1; or ii) determining if an allele or combination of alleles at loci in linkage disequilibrium with the nucleotide at position 2485 as numbered in SEQ ID NO:1 is/are present.

3. The method of claim 1, wherein the patient has a G allele at the nucleotide at position 2485 as numbered in SEQ ID NO:1, or an allele or combination of alleles at loci in linkage disequilibrium with the nucleotide at position 2485 as numbered in SEQ ID NO:1.

4. The method of claim 3, wherein the allele or combination of alleles in linkage disequilibrium with the nucleotide at position 2485 as numbered in SEQ ID NO:1 is selected from the group consisting of a nucleotide at position 2236 as numbered in SEQ ID NO:1, a nucleotide at position 2936 as numbered in SEQ ID NO:1, a nucleotide at position 5837 as numbered in SEQ ID NO:1, and combinations thereof.

5. The method of claim 1, wherein the patient is homozygous for the A allele at the nucleotide at position 2485 as numbered in SEQ ID NO:1, or an allele or combination of alleles at loci in linkage disequilibrium with the nucleotide at position 2485 as numbered in SEQ ID NO:1.

6. The method of claim 5, wherein the allele or combination of alleles in linkage disequilibrium with the nucleotide at position 2485 as numbered in SEQ ID NO:1, is selected from the group consisting of a nucleotide at position 2236 as numbered in SEQ ID NO:1, a nucleotide at position 2936 as numbered in SEQ ID NO:1, a nucleotide at position 5837 as numbered in SEQ ID NO:1, and combinations thereof.

7. The method of claim 1, wherein the first treatment regimen comprises administering to the patient a CCR-2 antagonist.

8. The method of claim 1, wherein the first treatment regimen comprises administering to the patient a CCL2 antagonist.

9. The method of claim 1, wherein the second treatment regimen comprises administering to the patient a treatment other than a CCR-2 antagonist or a treatment other than a CCL2 antagonist.

10. The method of claim 1, wherein the CCR-2 mediated disorder is selected from the group consisting of autoimmune disorders, cardiovascular disorders, inflammatory disorders, immune disorders, proliferative disorders, fibrotic disorders, viral infections, neurological disorders and metabolic disorders.

11. The method of claim 10, wherein the disorder is a cardiovascular disorder and the cardiovascular disorder is atherosclerosis.

12. A method of treating a patient having a CC-chemokine receptor 2 (CCR-2) mediated disorder, comprising:
i) selecting a patient based upon the patient having a G at a nucleotide at position 2485 as numbered in SEQ ID NO:1, or an allele or combination of alleles at loci in linkage disequilibrium with the nucleotide at position 2485 as numbered in SEQ ID NO:1, and
ii) administering to the patient a CCR-2 antagonist or a Chemokine Ligand CCL2 (CCL2) antagonist, to thereby treat the CCR-2 mediated disorder.

13. The method of claim 12, wherein the allele or combination of alleles at loci in linkage disequilibrium with the nucleotide at position 2485 as numbered in SEQ ID NO:1 is selected from the group consisting of a nucleotide at position 2236 as numbered in SEQ ID NO:1, a nucleotide at position 2936 as numbered in SEQ ID NO:1, a nucleotide at position 5837 as numbered in SEQ ID NO:1, and combinations thereof.

14. The method of claim 12, wherein the CCR-2 mediated disorder is selected from the group consisting of autoimmune disorders, cardiovascular disorders, inflammatory disorders, immune disorders, proliferative disorders, fibrotic disorders, viral infections, neurological disorders and metabolic disorders.

15. The method of claim 14, wherein the cardiovascular disorder is atherosclerosis.

16. A method of treating a patient having a CC-chemokine receptor 2 (CCR-2) mediated disorder, comprising:
i) selecting a patient based upon the patient having a homozygous A allele at a nucleotide at position 2485 as numbered in SEQ ID NO:1, or an allele or combination of alleles at loci in linkage disequilibrium with the nucleotide at position 2485 as numbered in SEQ ID NO:1, and
ii) administering to the patient a treatment other than a CCR-2 antagonist or other than a Chemokine Ligand CCL2 (CCL2) antagonist, to thereby treat the CCR-2 mediated disorder.

17. The method of claim 16, wherein the allele or combination of alleles at loci in linkage disequilibrium with the nucleotide at position 2485 as numbered in SEQ ID NO:1 is selected from the group consisting of a nucleotide at position 2236 as numbered in SEQ ID NO:1, a nucleotide at position 2936 as numbered in SEQ ID NO:1, a nucleotide at position 5837 as numbered in SEQ ID NO:1, and combinations thereof.

18. The method of claim 16, wherein the patient is administered i) an agent other than a CCR-2 antagonist or other than a CCL2 antagonist; ii) a CCR-2 antagonist in combination with an agent other than a CCR-2 antagonist; or iii) a CCL2 antagonist in combination with an agent other than a CCL2 antagonist.

19. The method of claim 16, wherein the CCR-2 antagonist or CCL2 antagonist is administered at an increased dose and/or an increased administration schedule.

20. The method of claim 16, wherein the CCR-2 antagonist or CCL2 antagonist is administered at a decreased dose and/or a decreased administration schedule.

21. The method of claim 16, wherein the CCR-2 mediated disorder is selected from the group consisting of autoimmune disorders, cardiovascular disorders, inflammatory disorders, immune disorders, proliferative disorders, fibrotic disorders, viral infections, neurological disorders and metabolic disorders.

22. The method of claim 2 i wherein the cardiovascular disorder is atherosclerosis.

23. The method of claim 22, wherein the treatment other than a CCR-2 antagonist or other than a CCL2 antagonist is a treatment regimen using HMG-CoA reductase inhibitors, Statins, Fibrates, ACE inhibitors, anti-hypertensives, anti-thrombotics, β blockers, anti-diabetes drugs or anti-obesity drugs.

24. The method of claim 23, wherein the HMG-CoA reductase inhibitor or Statin is selected from the group consisting of Atorvastatin, Cerivastatin, Fluvastatin, Lovastatin, Mevastatin, Pitavastatin, Pravastatin, Rosuvastatin, and Simvastatin.

25. A method of evaluating a patient having a CC-chemokine receptor 2 (CCR-2) mediated disorder for responsiveness or non-responsiveness to CCR-2 antagonist treatment regimens or Chemokine Ligand CCL2 (CCL2) antagonist treatment regimen, comprising:
i) determining whether a patient has a G allele at a nucleotide at position 2485 as numbered in SEQ ID NO:1 to determine the patient's genotype,
ii) recording the patient's genotype, and
iii) determining, recommending or selecting an appropriate treatment regimen based upon the presence or absence of a G allele.

26. The method of claim 25, wherein step iii) comprises determining whether to begin, continue, discontinue, change or alter a CCR-2 antagonist treatment regimen based upon the presence or absence of a G allele.

27. The method of claim 26, wherein the presence or absence of the G allele at the nucleotide at position 2485 as numbered in SEQ ID NO:1 is determined by i) determining if the G allele is present at the nucleotide at position 2485 as numbered in SEQ ID NO:1; or ii) determining if an allele or combination of alleles at loci in linkage disequilibrium with the nucleotide at position 2485 as numbered in SEQ ID NO:1 is/are present.

28. The method of claim 27, wherein the allele or combination of alleles at loci in linkage disequilibrium with the nucleotide at position 2485 as numbered in SEQ ID NO:1 is selected from the group consisting of a nucleotide at position 2236 as numbered in SEQ ID NO:1, a nucleotide at position 2936 as numbered in SEQ ID NO:1, a nucleotide at position 5837 as numbered in SEQ ID NO:1, and combinations thereof.

29. The method of claim 25, wherein the CCR-2 mediated disorder is selected from the group consisting of autoimmune disorders, cardiovascular disorders, inflammatory disorders, immune disorders, proliferative disorders, fibrotic disorders, viral infections, neurological disorders and metabolic disorders.

30. The method of claim 29, wherein the disorder is a cardiovascular disorder and the cardiovascular disorder is atherosclerosis.

31. A method of evaluating a patient having a CC-chemokine receptor 2 (CCR-2) mediated disorder for non-responsiveness to CCR-2 antagonist treatment regimens or a Chemokine Ligand CCL2 (CCL2) antagonist treatment regimen, comprising:
i) determining whether a patient is homozygous for the A allele at a nucleotide at position 2485 as numbered in SEQ ID NO:1 to determine the patient's genotype,
ii) recording the patient's genotype, and
iii) determining, recommending or selecting an appropriate treatment regimen based upon the presence or absence of a homozygous A allele.

32. The method of claim 31, wherein step iii) comprises determining whether to begin, continue, discontinue, change or alter a CCR-2 antagonist treatment regimen based upon the presence or absence of a homozygous A allele.

33. The method of claim 32, wherein the presence or absence of the homozygous A allele at the nucleotide at position 2485 as numbered in SEQ ID NO:1 is determined by i) determining if the homozygous A allele is present at the nucleotide at position 2485 as numbered in SEQ ID NO:1; or ii) determining if an allele or combination of alleles at loci in linkage disequilibrium with the nucleotide at position 2485 as numbered in SEQ ID NO:1 is/are present.

34. The method of claim 33, wherein the allele or combination of alleles at loci in linkage disequilibrium with the nucleotide at position 2485 as numbered in SEQ ID NO:1 is selected from the group consisting of a nucleotide at position 2236 as numbered in SEQ ID NO:1, a nucleotide at position 2936 as numbered in SEQ ID NO:1, a nucleotide at position 5837 as numbered in SEQ ID NO:1, and combinations thereof.

35. The method of claim 34, wherein the treatment regimen is selected from the group consisting of i) a treatment regimen other than a CCR-2 antagonist; ii) a CCR-2 antagonist in combination with an agent other than a CCR-2 antagonist; iii) a CCR-2 antagonist administered at an increased dose and/or an increased administration schedule; iv) a CCR-2 antagonist administered at a decreased dose and/or a decreased administration schedule; and v) no treatment regimen.

36. The method of claim 31, wherein the CCR-2 mediated disorder is selected from the group consisting of autoimmune disorders, cardiovascular disorders, inflammatory disorders, immune disorders, proliferative disorders, fibrotic disorders, viral infections, neurological disorders and metabolic disorders.

37. The method of claim 36, wherein the disorder is a cardiovascular disorder and the cardiovascular disorder is atherosclerosis.

38. The method of claim 37, wherein the treatment regimen is begun, continued, changed or altered to comprise a treatment other than a CCR-2 antagonist regimen and the treatment regimen comprises HMG-CoA reductase inhibitors, Statins, Fibrates, ACE inhibitors, anti-hypertensives, anti-thrombotics, β blockers, anti-diabetes drugs or anti-obesity drugs.

39. The method of claim 38, wherein the HMG-CoA reductase inhibitor or Statin is selected from the group consisting of Atorvastatin, Cerivastatin, Fluvastatin, Lovastatin, Mevastatin, Pitavastatin, Pravastatin, Rosuvastatin, and Simvastatin.

40. A method of selecting a treatment regimen for a patient having a CC-chemokine receptor 2 (CCR-2) mediated disorder, comprising:
i) determining whether the patient has a G allele at a nucleotide at position 2485 as numbered in SEQ ID NO:1,
ii) recording the patient's genotype, and
iii) selecting an appropriate treatment regimen based upon the presence or absence of a G allele at the nucleotide at position 2485 as numbered in SEQ ID NO:1.

41. The method of claim 40, wherein the presence or absence of the G allele at the nucleotide at position 2485 as numbered in SEQ ID NO:1 is determined by i) determining if the G allele is present at the nucleotide at position 2485 as numbered in SEQ ID NO:1; or ii) determining if an allele or combination of alleles at loci in linkage disequilibrium with the nucleotide at position 2485 as numbered in SEQ ID NO:1 is/are present.

42. The method of claim 41, wherein the allele or combination of alleles at loci in linkage disequilibrium with the nucleotide at position 2485 as numbered in SEQ ID NO:1 is selected from the group consisting of a nucleotide at position 2236 as numbered in SEQ ID NO:1, a nucleotide at position 2936 as numbered in SEQ ID NO:1 a nucleotide at position 5837 as numbered in SEQ ID NO:1, and combinations thereof.

43. The method of claim 40, wherein if the patient has a G allele at the nucleotide at position 2485 as numbered in SEQ ID NO:1, then the selected treatment regimen is a CCR-2 antagonist treatment regimen or a CCL2 antagonist treatment regimen.

44. The method of claim 40, wherein the CCR-2 mediated disorder is selected from the group consisting of autoimmune disorders, cardiovascular disorders, inflammatory disorders, immune disorders, proliferative disorders, fibrotic disorders, viral infections, neurological disorders and metabolic disorders.

45. The method of claim 44, wherein the disorder is a cardiovascular disorder and the cardiovascular disorder is atherosclerosis.

46. A method of selecting a treatment regimen for a patient having CC-chemokine receptor 2 (CCR-2) mediated disorder, comprising:
i) determining whether a patient is homozygous for the A allele at a nucleotide at position 2485 as numbered in SEQ ID NO:1,
ii) recording the patient's genotype, and
iii) selecting an appropriate treatment regimen based upon the presence or absence of a homozygous A allele at the nucleotide at position 2485 as numbered in SEQ ID NO:1.

47. The method of claim 46, wherein the presence or absence of the A allele at the nucleotide at position 2485 as numbered in SEQ ID NO:1 is determined by i) determining if the A allele is present at the nucleotide at position 2485 as numbered in SEQ ID NO:1; or ii) determining if an allele or combination of alleles at loci in linkage disequilibrium with the nucleotide at position 2485 as numbered in SEQ ID NO:1 is/are present.

48. The method of claim 47, wherein the allele or combination of alleles at loci in linkage disequilibrium with the nucleotide at position 2485 as numbered in SEQ ID NO:1 is selected from the group consisting of a nucleotide at position 2236 as numbered in SEQ ID NO:1, a nucleotide at position 2936 as numbered in SEQ ID NO:1, a nucleotide at position 5837 as numbered in SEQ ID NO:1, and combinations thereof.

49. The method of claim 46, wherein if the patient has a homozygous A allele at the nucleotide at position 2485 as numbered in SEQ ID NO:1, then the selected treatment regimen is selected from the group consisting of i) a treatment regimen other than a CCR-2 antagonist; ii) a treatment regimen other than a CCL2 antagonist; iii) a CCR-2 antagonist in combination with an agent other than a CCR-2 antagonist; iv) a CCL2 antagonist in combination with an agent other than a CCL2 antagonist; v) a CCR-2 antagonist administered at an increased dose and/or an increased administration schedule; vi) a CCL2 antagonist administered at an increased dose and/or an increased administration schedule; vii) a CCR-2 antagonist administered at a decreased dose and/or a decreased administration schedule; viii) a CCL2 antagonist administered at a decreased dose and/or a decreased administration schedule; and ix) no treatment regimen.

50. The method of claim 46, wherein the CCR-2 mediated disorder is selected from the group consisting of autoimmune disorders, cardiovascular disorders, inflammatory disorders, immune disorders, proliferative disorders, fibrotic disorders, viral infections, neurological disorders and metabolic disorders.

51. The method of claim 50, wherein the disorder is a cardiovascular disorder and the cardiovascular disorder is atherosclerosis.

52. The method of claim 51, wherein the treatment regimen comprises a treatment regimen using HMG-CoA reductase inhibitors, Statins, Fibrates, ACE inhibitors, anti-hypertensives, anti-thrombotics, β blockers, anti-diabetes drugs or anti-obesity drugs.

53. The method of claim 52, wherein the HMG-CoA reductase inhibitor or Statin is selected from the group consisting of Atorvastatin, Cerivastatin, Fluvastatin, Lovastatin, Mevastatin, Pitavastatin, Pravastatin, Rosuvastatin, and Simvastatin.

54. An article of manufacture comprising:
i) a pharmaceutical composition comprising a CC-chemokine receptor 2 (CCR-2) antagonist or a Chemokine Ligand CCL2 (CCL2) antagonist, and
ii) instructions for determining the appropriateness of use of said composition by determining a patient's genotype of a nucleotide at position 2485 as numbered in SEQ ID NO:1, or a nucleotide or allele in linkage disequilibrium with the nucleotide at position 2485.

55. An article of manufacture comprising:
i) reagents for making a determination of a patient's genotype at a nucleotide at position 2485 as numbered in SEQ ID NO:1, or a nucleotide or allele in linkage disequilibrium with the nucleotide at position 2485, and
ii) instructions for determining the appropriateness of use of a pharmaceutical composition comprising a CC-chemokine receptor 2 (CCR-2) antagonist or a Chemokine Ligand CCL2 (CCL2) antagonist on the basis of said determination.

56. An article of manufacture comprising:
i) a pharmaceutical composition comprising a CC-chemokine receptor 2 (CCR-2) antagonist or a Chemokine Ligand CCL2 (CCL2) antagonist,
ii) reagents for making a determination of a patient's genotype at a nucleotide at position 2485 as numbered in SEQ ID NO:1, or a nucleotide or allele in linkage disequilibrium with the nucleotide at position 2485; and
iii) instructions for determining the appropriateness of use of a pharmaceutical composition comprising a CCR-2 antagonist or a CCL2 antagonist on the basis of said determination.

57. The method of claim 7, 8, 12, 18, 25, 31, 43, 49, 54 or 56, wherein the CCR-2 antagonist or CCL2 antagonist is selected from the group consisting of a peptide, a polypeptide, an antibody or fragment thereof, and a non-protein molecule.

58. The method of claim 57, wherein the CCR-2 antagonist is a non-protein molecule selected from the group consisting of MK0812, INCB3284, SSR 150106, CCX915, INCB3344, and combinations thereof.

59. The method of claim 57, wherein the CCR-2 antagonist is an antibody or antigen binding fragment thereof.

60. The method of claim 59, wherein the antibody or antigen binding fragment thereof is selected from the group consisting of:
a) a monoclonal antibody;
b) a humanized antibody;
c) a human antibody;
d) a chimeric antibody;
e) an Fv fragment;
f) an Fab fragment;
g) an Fab′ fragment; and
h) an F(ab′)₂ fragment.

61. The method of claim 59, wherein the antibody or antigen binding fragment thereof is selected from the group consisting of MLN1202, 1D9, 8G2, MCPR-04, MCPR-05, or MCPR-06, and combinations thereof.


SEQUENCE LISTING (29 sequences)

SEQ ID NO: 1 - DNA, 11900 bp, Homo Sapiens
  exon (5001)...(5149): Exon 1
tgtagtcccagctactcgggaggctgaggcaggagaacctcttgaatccaggaggcgcaggttgcagtgagcagagatagtgccactgcactccagcctgggtgacagagtgagactctgtctcaaaaaaataaaataaaataaaaaatgcagactgtgattcagcaggtctgggttgaagcccagaactctctgataaattcaatggcacttaactacttggaggtcatggatgcctttgctaatctaatagaagctactgaccctctctccagaaaaatgcacaaaaacataaatgtggaagacaactcctgatggatctgggagcctatccaagggccacagacaagagtcctggtctggacaaaatgagctgctcagtattttcccacctggccagcatttcctatccaaagacaaatgttaaagttgttctagcagagccatgcaccagcagcagtatcatcacctgggaaccggttagcaatgcagaaccgcaggcccaccccaaacctacagtcagaatctctactttagcaagatcctaaggagatgggtaagcacattacaatttgcaacctttgtaagtttgcccaaaatgtgacccctccttcacccaccgatcgccaaggttcaaaaatctgcccaacccttgagcccatcttaaatgtaccatcacgagccttccctgggcccctcagctgggactctcaccgctctgtatctttctggttaatgcaattattctgttcccttagatgaccccagcacaggtgctaaaggagtcaacaaaaggctattgtcaaaaaagtgtttctgtctccactccatctgatctctgtttccctaagacctgcccatccccctctcccagttcggcaccttgaccccctcatcacactgctcaggccaccttgtacaatgcaagccccaaatgaggaaagcattttctcccccaatgtgtaacacgaaagtgctgtagagtggctcacgctgcctttagcctaagaatttatttaactcttacccccaacccacatcagtctcctccctctagggctcaggtgctaatctgtgagggctggctcagaagacaatctaaagaacaagcctcttgcttcctcaggcatcactactcctcaccaccatcacccccacccaccaactcaggccactactctttctgttctcatatgctatgcccatcgccacccctattcccatgctcaggagtattcttggctactgcatgcaattagacctggggcagatccaatccagaaagcaagaaatcttagatgctggaagcttggggtaagtactgatcagatttattcctaaattcagtcctactttccatggattcttactttagcatctcttctgaaaaggaagcatcatgtctaattcacttctccctccctgtgcagtcctctacctggtgctctgcacagggtatgtgctaattgtatgaatgttataataaagagatagtgcagtagatgacaaagggcactacattgagagcccagaaataagcaaaccagcacaaatgtagccattcgtcttctatctcaccttgagcctgtcactaacctgttcatggcctcagtctccccatcagagaaacaggtagatggtctctaaggtctcgttcattttctgacattctgtgaaaaattaaggaaagattttcatccttgacaggaaagggattgcagagtagcggccctgggaaaatgggctctattctacctggagctagcctggaggagaggccttgagtgggggttgtctagaaaggacatggtgagtgcagagctacggtgcatctctcttgaaggctgagtgaagggagcaccagcaagggagcctgcactaggtggggagggacaagtgaaccgcagaagttggtgggagcccaggcagtggcttcagatctttccagagagctcacttttacttcctctttttttcacccctgacactgagtgggagtctgcagcgatgaccaaggttcatgcagaggatcttagtggtggggtcagaccccgggaggaatgaagaaagcattattcaccaagaggagcttttccattctttatctatgagttgatagagaggaggccccggggtaactgaggattctggacagcatcagagcattgaccctcattttccccatagcccctctgggggcctttcccttgtgtgtccccaagcgagagtccaaccnaggtttgtgccagagcctaacccaggcttgtgccgagatgttcccagcacagccccatgtgagagctccctggctccgggcccagtatctggaatgcaggctccagccaaatgcattctcttctacgggatctgggaacttccaaagctgcctcctcagagtgggaatttccactcacttctctcacgccagcactgacctcccagcgggggagggcatcttttcttgacagagcagaagtgggaggcngacagctatcactttccagaagactttcttttctgattcatacccttcaccttccctgtgtttactgtctgatatatgcaaaggccaagtcactttccagagatgacaactccttcctgaagtagagacatgcttccaacactcagaagcctatgtgaacactcagccagcaaagctgggaagtttttctctgtgaccatgggctaattggtctccttctctggattgtggctttatcagataaaaacaagtggtcatgccacaggatgtctataagcccattgattctgggattctatgagtgatgctgatatgactaagccaggagagacttatttaaagatctcagcatctttcagcttgttaacctagagaaaacccgaagcatgactggattataaagggaaattgaatgcggtccaccaagttcatggtaaaggatgcactaacngattagagagaggtttcccctgatatgaggaaaacttcttggaagatgaggtgagatggcctaggaagaaattcctacacaaagttgcacagtctctagtcctggaaacattttattcattggataagaatggattgaggcatgagcagaggactgagacaaacacagagaagtttcaacactggttggggagaaaaggagtaactagtgagattcaggcagaacaagaataaggctcctcaagaggcacaagcaaagcagggctcgagttgatttgttctctcttcatcctgctttttgtaattccaccagagtctgaaatggccactccatagagtctctgctctgggattctccaggaaaccaatatccatcatgagacatcaagtctagtcccaggaagaagagattctggaatggaaacatcctgggtgggagtctcagcacatctactattctgtctgagttactggacaaataacttcagttttaacctaacgaaagctgggttggttggaggactgggcaggcagcgctggaaagtatgtcagcaccatacctgactccctgaatgcactcaacaatgccattactgaccacttactagaaataaaacagtcatttgttgaatacaacccgtttctttttacaagtgtagtgaaaagtgttttctttcaagaaaccccatgcatttatagacattgcctcagtgaccctttatgaaagaagtcactagtctttgtatgcccattgggcaagggcaccgcaaggctcagaaggaggaggcagtgggctaggagaatcgagagatcagaattttaaactcagcccagccattaacatgcctcaagtactcctatcatatttgtaagagacaacagttcactgaaatgaattctaaggtctttgggtttttatcagtgtgcttctgtagtttctgaggaaatctaaggcacaactgaggaatgaagtcaggctttccaattcccgaaatactcctccactgcttactcatgtcccttggaaattaagaaggaagccaggagaatagctgccataaccagggatgaacttcttgtccactgctgcctgctatgctagcaacagcctcctaactcataatgacttagccatgaggaatgtttctagattctcctttagctgtctgcccatttggaagatgctgaggacagagagaggacccaagcaggcaactagttggaggacttgtacacgtttccttccagcagtatgtcagagaggtggcagcccactggggacagggctgcctgggttctgtgctcgaggggaccttgagcaggctatttaacccttctgtgcctcagttgcctgatctataacatgaaaattagcaatccctactagataaagttggggaatttacagagttaatatttgtaaaggtctgagaatattcctggcagagtaagcactctgtgagtatgacactggcatttcttctgcagcactacatgctgtctatgcctttgtccaagtctgaaaccctagaactcttagaattcagttcaatgtttacacaatcctacagttctgctaggcttctatgatgctactattctgcatttgaatgagcaaatggatttaatgcattgtcagggagccggccaaagcttgagagctccttcctggctgggaggccccttggaatgtggcctgaaggtaagctggcagcgagcctgacatgctttcatctagtttcctcgcttccttccttttctgcagttttcgcttcagagaaagcagaatccttaaaaataaccctcttagttcacatctgtggtcagtctgggcttaatggcaccccatcctccccatttgctcatttggtctcagcagtgaatggaaaaagtgtctcgtcctgaccccctgcttccctttcctacttcctggaaatccacaggatgctgcatttgctcagcagatttaacagcccacttatcactcatggaagatccctcctcctgcttgactccgccctctctccctctgcccgctttcaataagaggcagagacagcagccagaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccaggtaaggccccctcttcttctccttgaaccacattgtcttctctctgagttatcatggaccatccaagcagacgtggtacccacagtcttgctttaacgctacttttccaagataaggtgactcagaaaaggacaaggggtgagcccaaccacacagctgctgctcggcagagcctgaactagaattccagctgtgaaccccaaatccagctccttccaggattccagctctgggaacacactcagcgcagttactcccccagctgcttccagcagagtttggggatcagggtaatcaaagagagggtgggtgtgtaggctgtttccagacacgctggagacccagaatctggtctgtgcttcattcaccttagcttccagagacggtgactctgcagaggtaatgagtatcagggaaactcatgaccaggcatagcctattcagagtctaaaaggaggctcatagtggggctccccagctgatcttccctggtgctgatcatctggattattggtccgtcttaatgacacttgtaggcattatctagctttaacagctcctccttctctctgtccattatcaatgttatatacccattttacagcataggaaactgagtcattgggtcaaagatcacattctagctctgaggtataggcagaagcactgggatttaatgagctctttntcttctcctgcctgccttttgctttttcctcatgactcttttctgctcttaagatcagaataatccagttcatcctaaaatgctttttctttgtggtttattttccagatgcaatcaatgccccagtcacctgctgttataacttcaccaataggaagatctcagtgcagaggctcgcgagctatagaagaatcaccagcagcaagtgtcccaaagaagctgtgatgtgagttcagcacaccaaccttccctggcctgaagttcttccttgtggagcaagggacaagcctcataaacctagagtcagagagtgcactatttaacttaatgtacaaaggttcccaatgggaaaactgaggcaccaagggaaaaagtgaaccccaacatcactctccacctgggtgcctattcagaacaccccaatttctttagcttgaagtcaggatggctccacctggacacctataggagcagtttgccctgggttccctccttccacctgcgttcctcctctagctcccatggcagccctttggtgcagaatgggctgcacttctagaccaaaactgcaaaggaacttcatctaactctgtcctccctccccacagcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaacctgacttccagtgttttcttgaaggaaattacaaagctgagagtatgagcttggtggtgacaaaggaacatgatttcagagggtggggcttacattttgaaggaatgggaaagtggattggccccggtcttctccactgggtggtctcctctgagtctccgtagaagaatctttatggcaggccagttaggcattaaagcaccacccttccagtcttcaacataagcagcccagagtccaatgaccctggtcacccatttagcaagagcccaacccccattccttttctcacagaccctgacccctgcatgcaattcttcccttaacatattgcaactgccccctaactgggctacccaccccccaatctgtacctctccaattaataccccaacctggagtaatacagacactgccagtattaggaaataaggaaagagttaatcaccatagataagatgattagattgaagtttcatagagatgatgagacctgaacttattatttatgaatgaagaaggcttttctaggaaaattataggatcattaagaaaggagaaggaagagtgggagcaaatacctggaggtagaaatggtgatgatgtgtacatcaagcagggagaaaaccaatgaaccagatgcgaattcgggcccacaccaatgtcaagggatgacaattagaaaggaaggttgagtcaagggatttgaatgttagggtgaaaagttactactcaactctgtaggttaaaaggaaacgttgagaatcttcagtccaatgaggagggatgtgccatgtttagagattcagagataagtttcaggaaatgtaacttatagattttatacatacacagagaaatacggactagtgagaagctattgccatggtccaagcaagagatgatgaaggcctaaatatggagccaaagaggcagcaatgaagaatgagccatgcagggtgaaatgctgcatgttgtaaatggaggagaaagacctgtgacttcagatatgaaaacctcatcttcaacccacattttaagggggcagcttccctgaaaccagaatgtgtttccctccattactatacccccatcccaatctcaggcacctggaatcatccatttaaacagatgagccttctattcctaaatagccacctgaagtgtgtattcctttgcatgatatttgtcccacctaaagcattcgacctgcctgggcacccacaccacgccaacactcaggaaagcagatgtcttgctctgttgaataaactgcatggttcttaacttcccagtctggtggggaaatgaccactgtgtcaacctagagcaggcagtgcttttggcagcatgaggtgctggggacaactttgactggcaagaagcacactcaggttctcaccccgcatccagcgctgactcgctttgtcagtcaagacaggtcagatattctgagcctacatcgatcatacaggtatgataatgtgttacaaataggaacccagaggaaaggttccctttcggatctgggagcacatctgttggaaaacttccatttctactaactggagttgcagagggagagaagggattctgcttctacattcctgagccagtccagggtccctgaatcagactaccgaatcccttcaaagctccaagtaccctgatatatcagtcagcagacaatttattgacagctatttagaaaactcactgaccctcactccaggtcaagcagcgtcccctgcctctcctctacccctacattccctggccttgatcaccagtcaggagtgaaatctcaaattgcagtagatgccaagaggcaaaaagagaatagaatgcaaacaaatgagacctcatcatacggcttccgagcagcaaccttttgacgccaggcagatttgaggcagacagtctgggaggagaggaggcagagaaaggggggatccacatgctcaaaccccaaattaatctgcttacattccccttgcaggccacatctcttcattttcaggaagtcttgactccatactgttttccacccaagcatggaattcctttcatgatgaaactgaacacagggcattggcagtggtgagactctgttttagaagaaagtgccaagtgcaatgcattcatttcctgttgctgccaacaatcagttccaggaaatctaggctttttatgtcatgctcaaaattcttccagcctatgctcattattcaaatccaaagccacatccacatctgtaggtgttagttacagaagcaccatatttccaggtaccaaaatctgtattagtttcttattgttactgtaacaaattcccataagcttaatggcttaaaacaacaaaaatacattgtcttatagttctggaggtcagagttatcatgggtccatagggctatattcattccttccagaggttctatgggagaatccattttcttgcttttttccaactgctagaggctacctcaattccttaactcatggtctcttcctgtgtcttcaaaaacctctctctctctctctctcactccttttctttcatagtcacatcttgtctctgactctgactttcctgccttcatcttaaaaggtgattatattgtgattatgctgagcccatttggataatccaggataacatcttcatctcaagattcttaagttaatcacatttataaaatttcttttgctatgtcaggtagcatacttatagagcatagggattcgattaggatgtaggctttttttttttttttttttttttttggaaatggagtctcgctctattgcccaggctggagtgcagtagcacaatctcagctcactgcaacctctgtctcctgggttcaagtgattctcctgcctcagcctcccaagtaactgtgattacagatgtgcaccaccacacctggctaatttttgtatttttagtagagatggggttttaccatgttggccaggctggtctcgaactcctgacctcaggtgatccacccgcctcagactcccagagtacaggtgtgagacaccacgcctggctacaaggatgtgagcatctttgggaggggagcactactgggacaatcacagccaggagccaggtgaagatttacaataggtagttggacactaagacatggcctttgagatctgtatctggtttggtgactagattgtcaggtaaacttttgcaagtcaccttttatctatgggttcataggctgtggatttaatttgtgcattaaatgcttcctaagtgacctaaggtatattggagtcacctgaggtctttccaaaacatcacaacatcctcaaagattctgatttagaatgttacaatgcagggaaggctcctcttcccattgctgtattttacgagttccctagaagatggatactggtgcacagatgagtttgatgccatgtgctcctcagttctctcatattcaggtcattggagccaacagccagtcctggtaacctctgacttgggttttcatagtgtcctttgttctgacctagccttgaccaatttacaatcaggaacaaaagggagcatagagtcgagacatttaggacattgtaaagtttgaaatacaactgttcaccatcccaggcagtggaatggaatttttaaaaatgatttgaacaacaaaatatccataaaacctttcagtttgacagagtggcttcaggaagagagtgggttaacaatgtggcattcccattctagcccggcagcctgatcctaaaagacagttatgggtcgggtgtggaggaagcacagaaatatgtcaagcactgcgagtagaaagagattttacatgtctttactggcagattggaaaagtgctagtaattttttgagaaaattatgttgccaggaaaatcagaagccaagactgaaaaagcctttgactattctatctataaaacaacttccgaatcttccatgagctagaaacaagccatgacagccactgaattaggagggcatgtccactgagttccatgtacagtatggccaaggagaagaaatggacaagcaagggcctcacagaaggtggatctggggccccggacaaggaagcccttcctgcaagtgagctcagagcccctgttcctgttccaggaagagtcccttttgatgtctgcccagcagtatatcagaattgctacaagtcagtcattgctgtgtgtcccccagtcttccttgtccaatgagaatgtttactttaagcatcttgttcctattccaccttgtaaatgagtgtttaaattgagcagataaattgttcattttttaaaagtactgtcaatctttcatttggaggctataaaattcatttccataaggtatcattatctcttttttaaagttttcaattttgaaaaattttggttaaaaaatgcataccatgaaatgtatcatatttaccatttttaagtgtatacttcagtattgttaatgttcatatacttaacattgttgtgcaatagatttccatcttgtaaatccatcttgtaaaactgaaactgcatactcattaaacaatacctgctcatttcctccgctgcccagccccggcaaccaccattctactttcctcttctatgaatttgactactttaggtaccttctgtaagtgggattatacaatatgtgtcttttggtgaatggttattttatttagcataatgttctcaaggttcatttatgttaaggcatgtgtcaaaattcccttcttttttaaggttgaataatattccattgtatgtatataccacattttgtttatctgttcattcagtgatggacagctgggttgttttcatctcgtggttattgtgcaggatgctgttctgaatgtggatatgcaaatgtctcttccagattctgctttcagttcctttggatatatacccacaagtgggattgctgcatcatttg

SEQ ID NO: 2 - protein, 112 aa, Artificial Sequence
  Humanized Variable Light Chain
DVVMTQSPLSLPVTLGQPASISCKSSQSLLDSDGKTFLNWFQQRPGQSPRRLIYLVSKLDSGVPDRFSGSGSGTDFTLKISRVEAEDVGVYYCWQGTHFPYTFGQGTRLEIK

SEQ ID NO: 3 - protein, 117 aa, Artificial Sequence
  Humanized Variable Heavy Chain
EVQLVESGGGLVKPGGSLRLSCAASGFTFSAYAMNWVRQAPGKGLEWVGRIRTKNNNYATYYADSVKDRFTISRDDSKNTLYLQMNSLKTEDTAVYYCTTFYGNGVWGQGTLVTVSS

SEQ ID NO: 4 - protein, 330 aa, Artificial Sequence
  Human IgG1-FcRmut Protein
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 107 aa, Artificial Sequence [sequence omitted]

SEQ ID NO: 6 - DNA, 333 bp, Artificial Sequence
  Humanized Light Chain
gtagtgatgacccagtctccactctccttgcccgttacccttggacagccagcctccatctcttgcaagtcaagtcagagcctcttagatagtgatggaaagacatttttgaattggtttcagcagaggccaggccagtctccaaggcgcctaatctatctggtgtctaaactggactctggagtccctgacaggttcagcggcagtggatcagggacagatttcacactgaaaatcagcagagtggaggctgaggatgttggagtttattattgctggcaaggtacacattttccgtacacgttcggacaagggacccgactggagatcaag

SEQ ID NO: 7 - DNA, 351 bp, Artificial Sequence
  Humanized Heavy Chain
gaggtgcaattggttgagtctggaggaggattggtgaagcctggggggtcattgagactctcatgtgcagcctctggattcactttcagtgcctacgccatgaactgggtccgccaggctccaggaaagggtttggaatgggttggccgcataagaactaaaaataataattatgcaacatattatgccgattcagtgaaagacagattcaccatctccagagatgattcaaaaaacacgctctatctgcaaatgaacagcttgaaaactgaggacacagccgtgtattactgtaccaccttttacggtaacggtgtctggggccaggggaccctggtcaccgtcagctca

SEQ ID NO: 8 - DNA, 1002 bp, Artificial Sequence
  Human IgG1-FcRmut DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataatctagagca

SEQ ID NO: 9 - DNA, 321 bp, Artificial Sequence
  Human C Kappa DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgaccctgagcaaagcagactacgagaaacacaaagtctacgcctgcgaagtcacccatcagggcctgagctcgcccgtcacaaagagcttcaacaggggagagtgt

SEQ ID NO: 10 - DNA, 20 bp, Artificial Sequence
  Primer
tgaccctcattttccccata

SEQ ID NO: 11 - DNA, 20 bp, Artificial Sequence
  Primer
cacagggaaggtgaagggta

SEQ ID NO: 12 - DNA, 20 bp, Artificial Sequence
  Primer
gctcatgcctcaatccattc

SEQ ID NO: 13 - DNA, 20 bp, Artificial Sequence
  Primer
taggcagaagcactgggatt

SEQ ID NO: 14 - DNA, 20 bp, Artificial Sequence
  Primer
tggaaattcccactctgagg

SEQ ID NO: 15 - DNA, 20 bp, Artificial Sequence
  Primer
aaagctgcctcctcagagtg

SEQ ID NO: 16 - DNA, 20 bp, Artificial Sequence
  Primer
agagaaaacccgaagcatga

SEQ ID NO: 17 - DNA, 20 bp, Artificial Sequence
  Primer
gcattgattgcatctggaaa

SEQ ID NO: 18 - DNA, 18 bp, Artificial Sequence
  Probe
ccaagcgagagtccaacc

SEQ ID NO: 19 - DNA, 16 bp, Artificial Sequence
  Probe
gtcttctggaaagtga

SEQ ID NO: 20 - DNA, 17 bp, Artificial Sequence
  Probe
gaaacctctctctaatc

SEQ ID NO: 21 - DNA, 17 bp, Artificial Sequence
  Probe
gatttaatgagctcttt

SEQ ID NO: 22 - DNA, 16 bp, Artificial Sequence
  Probe
tccaaccaaggtttgt

SEQ ID NO: 23 - DNA, 16 bp, Artificial Sequence
  Probe
gaaagtgatagctgtc

SEQ ID NO: 24 - DNA, 19 bp, Artificial Sequence
  Probe
tctctaatcagttagtgca

SEQ ID NO: 25 - DNA, 15 bp, Artificial Sequence
  Probe
gctctttctcttctc

SEQ ID NO: 26 - DNA, 16 bp, Artificial Sequence
  Probe
tccaacccaggtttgt

SEQ ID NO: 27 - DNA, 16 bp, Artificial Sequence
  Probe
gaaagtgacagctgtc

SEQ ID NO: 28 - DNA, 19 bp, Artificial Sequence
  Probe
tctctaatctgttagtgca

SEQ ID NO: 29 - DNA, 15 bp, Artificial Sequence
  Probe
gctctttgtcttctc